(19) United States
(12) Patent Application Publication
(10) Pub. No.: US 2024/0115725 A1
(43) Pub. Date: Apr. 11, 2024

(54) Anti-EGFR Antibody-Drug Conjugates

(71) Applicant: AbbVie Inc., north chicago, IL (US)

(72) Inventors: Erwin R. Boghaert, Pleasant Prairie, WI (US); Andrew C. Phillips, Libertyville, IL (US); Andrew J. Souers, Libertyville, IL (US); Kamel Izeradjene, Gurnee, IL (US); John E. Harlan, Lake Zurich, IL (US)

(21) Appl. No.: 18/506,459

(22) Filed: Nov. 10, 2023

Related U.S. Application Data
(63) Continuation of application No. 18/160,231, filed on Jan. 26, 2023, now abandoned.
Application No. 18/160,231 is a continuation of application No. 17/580,134, filed on Jan. 20, 2022, now Pat. No. 11,759,527.
(60) Provisional application No. 63/139,766, filed on Jan. 20, 2021.

(51) Int. Cl. A61K 47/68; A61P 35/00; C07K 16/28

(52) U.S. Cl. CPC A61K 47/6849; A61K 47/6803; A61P 35/00; C07K 16/2863; A61K 2039/505

(57) ABSTRACT

The present disclosure relates to anti-Epidermal Growth Factor Receptor (EGFR) antibody drug conjugates (ADCs) which inhibit Bcl-xL, including compositions and methods using such ADCs, and methods for making such ADCs.

### FIELD OF THE INVENTION

The present application pertains to novel anti-Epidermal Growth Factor Receptor (EGFR) antibody drug conjugates (ADCs) which inhibit Bcl-xL, including compositions and methods using such ADCs, and methods for making such ADCs.

### SEQUENCE LISTING

The contents of the electronic sequence listing (ABV12608USC1_ST26.xml; Size: 48,764 bytes; and Date of Creation: Jan. 25, 2023) is herein incorporated by reference in its entirety.

### BACKGROUND

The human epidermal growth factor receptor (also known as HER-1 or Erb-B1 and referred to herein as “EGFR”) is a 170 kDa transmembrane receptor encoded by the c-erbB protooncogene. (Modjtahedi et al., Br. J. Cancer 73:228-235 (1996); Herbst and Shin, Cancer 94:1593-1611 (2002)). SwissProt database entry P00533 provides the sequence of human EGFR.

Ligand binding by EGFR triggers receptor homo-and/or heterodimerization and autophosphorylation of key cytoplasmic residues and MUCl. Phosphorylated EGFR activates complex downstream signaling cascades. Overexpression of EGFR has been reported in numerous human malignant conditions and associated with poor prognosis with patients. (Herbst and Shin, Cancer 94:1593-1611 (2002); and Modjtahedi et al., Br. J. Cancer 73:228-235 (1996)).

Antibody drug conjugates represent a class of therapeutics comprising an antibody conjugated to a cytotoxic drug via a chemical linker. Designing ADCs against EGFR has been challenging, because of cutaneous EGFR expression and the known skin toxicity of EGFR-directed antibodies. Anti-EGFR antibodies and antibody-drug conjugates are also described in U.S. Pat. No. 9,493,568 and U.S. Patent Application Publication No. 2019/0343961, which are incorporated by reference herein in their entireties. A first generation of EGFR ADCs was depatuxizumab mafodotin, which uses the maleimidocaproyl linker and microtubule cytotoxin monomethyl auristatin F (MMAF). However, patients receiving depatux-m have experienced frequent ocular side effects (e.g., dry eyes, blurry vision, eye pain, photophobia, keratitis, corneal deposits, and watery eyes).

Given the ocular toxicity of depatux-m, a second-generation ADC targeting EGFR, losatuxizumab vedotin, was conjugated to a different toxin. The antibody component of this ADC, losatuxizumab, was affinity maturated so that it had a higher affinity for EGFR (both wild-type and mutant) compared with depatux-m. However, the relatively high frequency of infusion reactions necessitated the early closure of the losatuxizumab vedotin phase 1 trial. See Cleary et al., Investigational New Drugs 38, 1483-1494 (2020).

Accordingly, there is a need for the development of new anti-EGFR ADCs, and in particular EGFR ADCs that can selectively deliver Bcl-xL to target cancer cells (e.g., EGFRvIII expressing cells), represents a significant discovery.

### SUMMARY

One aspect pertains to an anti-human epidermal growth factor receptor (hEGFR) antibody drug conjugate comprising the structure of Formula (I) conjugated to an antibody Ab:

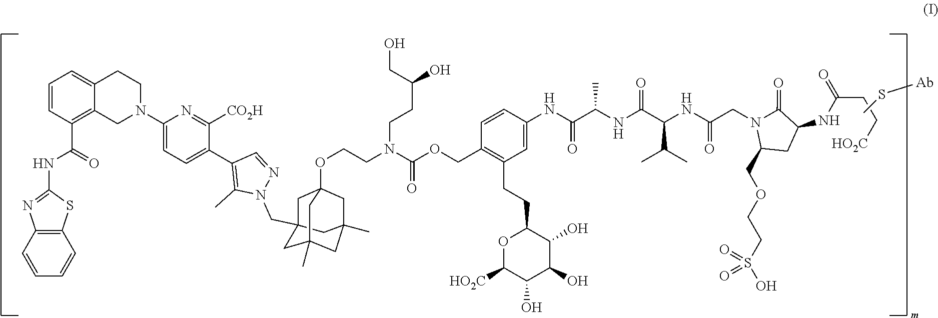

wherein Ab is an IgG1 anti-hEGFR antibody comprising a heavy chain comprising the amino acid sequence set forth as SEQ ID NO: 1 and a light chain comprising the amino acid sequence set forth as SEQ ID NO: 5; and wherein m is 2. In embodiments, the structure of Formula (I) is conjugated to the antibody Ab through C220 of the heavy chain (i.e., C219 of SEQ ID NO: 1). In embodiments, the present disclosure provides methods of using the anti-EGFR antibody drug conjugate for treating non-small cell lung cancer. In embodiments, the present disclosure provides pharmaceutical compositions comprising said anti-hEGFR antibody drug conjugate.

Another aspect pertains to an anti-human Epidermal Growth Factor Receptor antibody-drug conjugate comprising the structure of formula (II) conjugated to an antibody Ab:

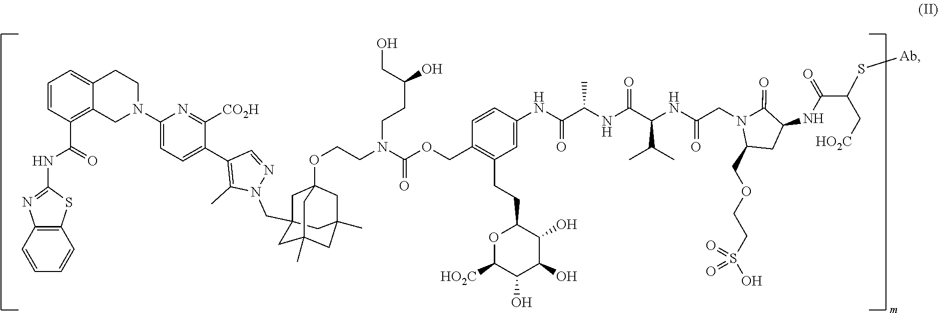

wherein Ab is an IgG1 anti-human epidermal growth factor receptor antibody comprising a heavy chain comprising the amino acid sequence set forth as SEQ ID NO: 1 and a light chain comprising the amino acid sequence set forth as SEQ ID NO: 5, and wherein m is 2. In embodiments, the structure of Formula (II) is conjugated to the antibody Ab through C220 of the heavy chain (i.e., C219 of SEQ ID NO: 1). In embodiments, the present disclosure provides methods of using the anti-EGFR antibody drug conjugate for treating non-small cell lung cancer. In embodiments, the present disclosure provides pharmaceutical compositions comprising said anti-hEGFR antibody drug conjugate.

Another aspect pertains to an anti-human Epidermal Growth Factor Receptor antibody-drug conjugate comprising the structure of formula (III) conjugated to an antibody Ab:

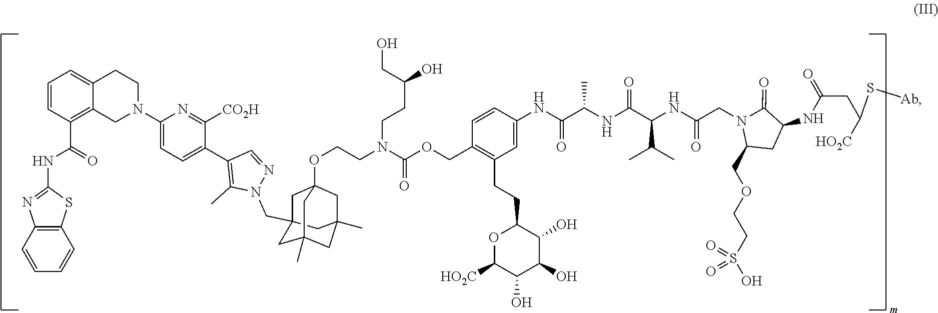

wherein Ab is an IgG1 anti-human epidermal growth factor receptor antibody comprising a heavy chain comprising the amino acid sequence set forth as SEQ ID NO: 1 and a light chain comprising the amino acid sequence set forth as SEQ ID NO: 5; and wherein m is 2. In embodiments, the structure of Formula (III) is conjugated to the antibody Ab through C220 of the heavy chain (i.e., C219 of SEQ ID NO: 1). In embodiments, the present disclosure provides methods of using the anti-EGFR antibody drug conjugate for treating non-small cell lung cancer. In embodiments, the present disclosure provides pharmaceutical compositions comprising said anti-hEGFR antibody drug conjugate.

Another aspect pertains to a method of producing an antibody drug conjugate (ADC) comprising a step of conjugating a monoclonal human IgG₁ anti-EGFR antibody with a synthon comprising the structure (AAA):

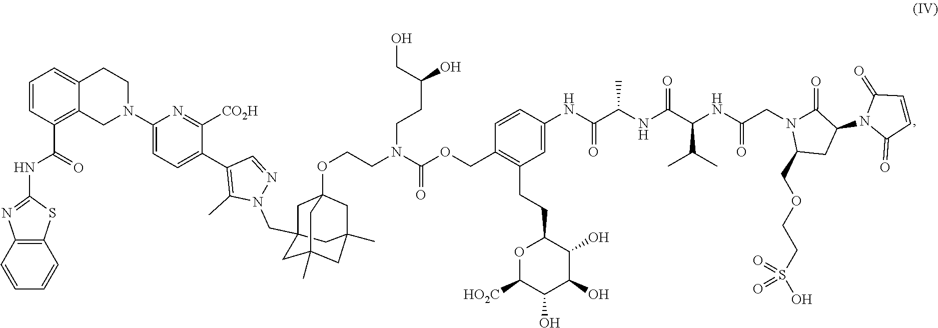 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CC[C@H](O)CO)C(=O)OCc1ccc(NC(=O)[C@H](C)NC(=O)[C@@H](NC(=O)CN4C(=O)[C@@H](N5C(=O)C=CC5=O)C[C@H]4COCCS(=O)(=O)O)C(C)C)cc1CC[C@@H]1O[C@H](C(=O)O)[C@@H](O)[C@H](O)[C@H]1O)(C3)C2]

to form an antibody-drug conjugate comprising a drug-linker conjugated to the anti-EGFR antibody, wherein the anti-EGFR antibody comprises a heavy chain comprising the amino acid sequence set forth as SEQ ID NO: 1 and a light chain comprising the amino acid sequence set forth as SEQ ID NO: 5. In embodiments, the drug-linker is conjugated to the antibody through C220 of the heavy chain (i.e., C219 of SEQ ID NO: 1).

Another aspect pertains to a process for the preparation of an antibody drug conjugate (ADC) according to Formula (I):

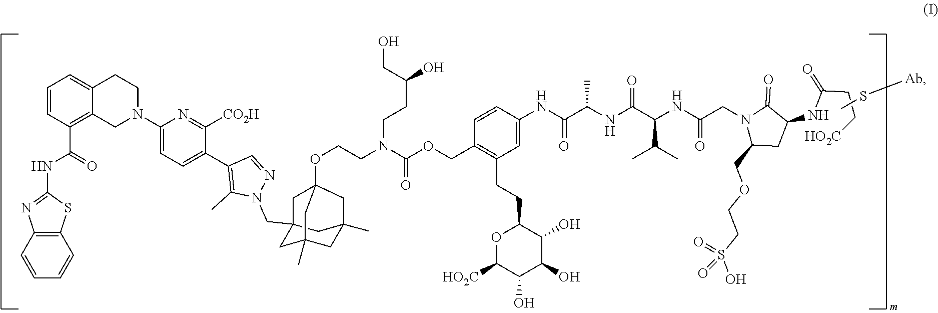

wherein Ab is an IgG1 anti-human epidermal growth factor receptor antibody comprising a heavy chain comprising the amino acid sequence set forth as SEQ ID NO: 1 and a light chain comprising the amino acid sequence set forth as SEQ ID NO: 5; and

m is 2;

the process comprising:

treating an antibody in a buffered aqueous solution with an effective amount of a disulfide reducing agent for about 16-24 hours;

adding to the reduced antibody solution a solution of dimethyl acetamide comprising a synthon having the following structure:

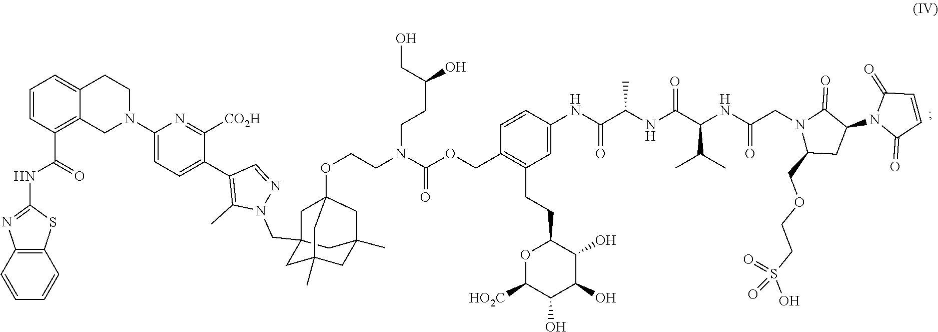 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CC[C@H](O)CO)C(=O)OCc1ccc(NC(=O)[C@H](C)NC(=O)[C@@H](NC(=O)CN4C(=O)[C@@H](N5C(=O)C=CC5=O)C[C@H]4COCCS(=O)(=O)O)C(C)C)cc1CC[C@@H]1O[C@H](C(=O)O)[C@@H](O)[C@H](O)[C@H]1O)(C3)C2]

allowing the reaction to run to form the ADC;

wherein the mass is shifted by 18±2 amu for each hydrolysis of a succinimide to a succinamide as measured by electron spray mass spectrometry; and

wherein the ADC is optionally purified by hydrophobic interaction chromatography (HIC). In embodiments, the buffered aqueous solution is a buffered aqueous solution of about pH 7.4. In embodiments, the antibody is treated with the disulfide reducing agent in the buffered aqueous solution at about 4° C. for about 16-24 hours. In embodiments, the reduced antibody is added to the solution of dimethyl acetamide comprising the synthon, and the reaction is allowed to run for about 60 minutes to form the ADC. In embodiments, after the ADC is formed, the reaction is quenched with about 2 equivalents of N-acetyl-L-cysteine. In embodiments, the process further comprises purifying with hydrophobic interaction chromatography (HIC). In embodiments, the process further comprises hydrolyzing the succinimide with a pH buffer, such as a pH buffer of about 8-9. In embodiments, the process further comprises purifying with tangential flow filtration (TFF). In embodiments, the linker-drug is conjugated to the anti-EGFR antibody Ab through C220 of the heavy chain (i.e., C219 of SEQ ID NO: 1).

Another aspect pertains to an antibody-drug conjugate (ADC) prepared by a method comprising a step of conjugating a monoclonal human IgG₁ anti-EGFR antibody with a synthon comprising the structure:

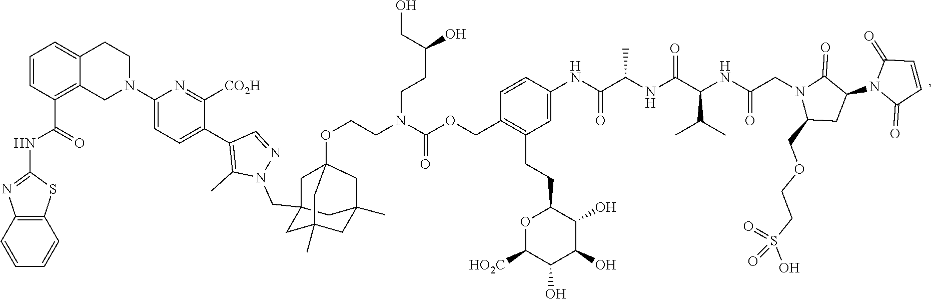 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CC[C@H](O)CO)C(=O)OCc1ccc(NC(=O)[C@H](C)NC(=O)[C@@H](NC(=O)CN4C(=O)[C@@H](N5C(=O)C=CC5=O)C[C@H]4COCCS(=O)(=O)O)C(C)C)cc1CC[C@@H]1O[C@H](C(=O)O)[C@@H](O)[C@H](O)[C@H]1O)(C3)C2]

wherein the antibody comprises a heavy chain comprising the amino acid sequence set forth as SEQ ID NO: 1 and a light chain comprising the amino acid sequence set forth as SEQ ID NO: 5. In embodiments, the drug-linker is conjugated to the anti-EGFR antibody through C220 of the heavy chain (i.e., C219 of SEQ ID NO: 1).

Another aspect pertains to a process for the preparation of an antibody drug conjugate (ADC) according to Formula (I):

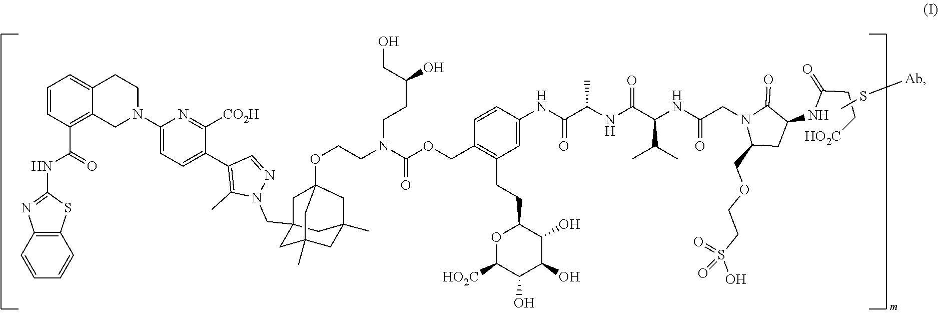

wherein Ab is an IgG1 anti-human epidermal growth factor receptor antibody comprising a heavy chain comprising the amino acid sequence set forth as NO: 1 and a light chain comprising the amino acid sequence set forth as SEQ ID NO: 5; and

m is 2;

the process comprising:

treating an antibody in a buffered aqueous solution of about pH 7.4 with an effective amount of a disulfide reducing agent at about 4° C. for about 16-24 hours;

allowing the reduced antibody solution to warm to ambient temperature;

adding to the reduced antibody solution a solution of dimethyl acetamide comprising a synthon having the following structure:

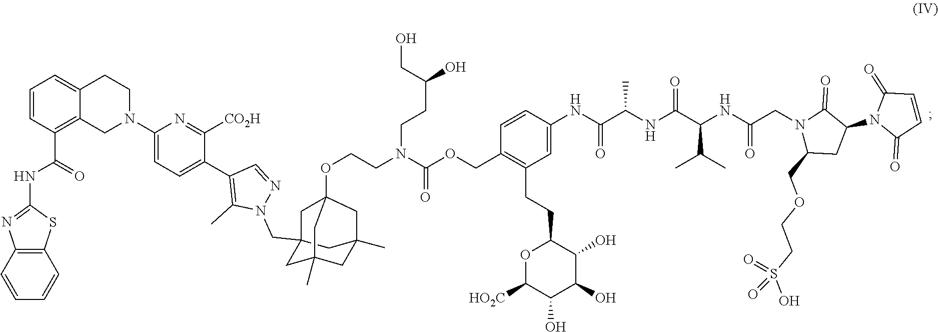 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CC[C@H](O)CO)C(=O)OCc1ccc(NC(=O)[C@H](C)NC(=O)[C@@H](NC(=O)CN4C(=O)[C@@H](N5C(=O)C=CC5=O)C[C@H]4COCCS(=O)(=O)O)C(C)C)cc1CC[C@@H]1O[C@H](C(=O)O)[C@@H](O)[C@H](O)[C@H]1O)(C3)C2]

allowing the reaction to run for about 60 minutes to form the ADC;

quenching with about 2 equivalents of N-acetyl-L-cysteine;

purifying with hydrophobic interaction Chromatography (HIC);

purifying with tangential flow filtration (TFF);

hydrolyzing the succinimide with a pH buffer of about 8-9; and

purifying with tangential flow filtration (TFF);

wherein the mass is shifted by 18±2 amu for each hydrolysis of a succinimide to a succinamide as measured by electron spray mass spectrometry; and

wherein the ADC is optionally purified by hydrophobic interaction chromatography. In embodiments, the linker-drug is conjugated to the anti-EGFR antibody through C220 of the heavy chain (i.e., C219 of SEQ ID NO: 1).

### BRIEF DESCRIPTION OF THE DRAWINGS

FIG. 1 shows binding of AM2-AAA, AM2, and MSL109 hIgG1, to A-431 (human epidermoid carcinoma) and NCI-H1650 (non-small cell lung cancer) as assessed by FACS.

FIG. 2 shows AM2-AAA disrupts BIM-Bcl-xL complexes and promotes caspase activation and disruption of Bcl-xL-BIM complexes in (A) A-431 (human epidermoid carcinoma) and (B) NCI-H1650 (non-small cell lung cancer) cells following treatment with AM2-AAA, MSL109 hIgG-AAA or AM2.

FIG. 3 shows inhibition of EBC-1 (human lung squamous cell carcinoma) and H441 (human lung adenocarcinoma) xenograft growth after treatment with AM2B-AAA, AM7-AAA, DTX and combinations of DTX with AM2B-AAA or AM7-AAA. Although AM7 is a higher affinity antibody than AM2B, AM2B-AAA achieved similar efficacy to AM7-AAA in preclinical efficacy models.

### BRIEF DESCRIPTION OF THE SEQUENCE LISTING

Incorporated herein by reference in its entirety is a Sequence Listing entitled, “ABV12608USO1 Sequence Listing_ST25.txt”, comprising SEQ ID NO: 1 through SEQ ID NO: 30, which includes the amino acid sequence disclosed herein. The Sequence Listing has been submitted herewith in ASCII text format via EFS. The Sequence Listing was first created on Jan. 13, 2022 and is 71,052 bytes in size.

### DETAILED DESCRIPTION

Various aspects of the present disclosure relate to new anti-EGFR antibody-drug conjugates (ADCs, also called immunoconjugates), and pharmaceutical compositions thereof. In particular, the present disclosure describes new anti-EGFR ADCs comprising Bcl-xL inhibitors, synthons useful for synthesizing the ADCs, compositions comprising the ADCs, methods of making the ADCs, and various methods of using the ADCs.

As will be appreciated by skilled artisans, the various Bcl-xL inhibitors, ADCs and/or ADC synthons described herein may be in the form of salts, and in certain embodiments, particularly pharmaceutically acceptable salts. The compounds of the present disclosure that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion.

In the disclosure herein, if both structural diagrams and nomenclature are included and if the nomenclature conflicts with the structural diagram, the structural diagram controls.

### Definitions

In order that the invention may be more readily understood, certain terms are first defined. In addition, it should be noted that whenever a value or range of values of a parameter are recited, it is intended that values and ranges intermediate to the recited values are also intended to be part of this invention. Further, unless otherwise defined herein, scientific and technical terms used in connection with the present disclosure shall have the meanings that are commonly understood by those of ordinary skill in the art.

The term “anti-Epidermal Growth Factor Receptor (EGFR) antibody” as used herein, refers to an antibody that specifically binds to EGFR. An antibody “which binds” an antigen of interest, i.e., EGFR, is one capable of binding that antigen with sufficient affinity such that the antibody is useful in targeting a cell expressing the antigen. In embodiments, the antibody specifically binds to human EGFR (hEGFR). Examples of anti-EGFR antibodies are disclosed below. Unless otherwise indicated, the term “anti-EGFR antibody” is meant to refer to an antibody which binds to wild type EGFR or any variant of EGFR, such as EGFRvIII.

The amino acid sequence of wild type human EGFR is provided below as SEQ ID NO: 15, including the signal peptide (amino acid residues 1-24), and the amino acid residues of the extracellular domain (ECD, amino acid residues 25-645). A truncated wild type ECD of the EGFR (also referred to herein as EGFR(1-525)) corresponds to SEQ ID NO: 16 and is equivalent to amino acids 1-525 of SEQ ID NO: 15. The mature form of wild type EGFR corresponds to the protein without the signal peptide, i.e., amino acid residues 25 to 1210 of SEQ ID NO: 15.

The amino acid sequence of the ECD of human EGFR is provided below as SEQ ID NO: 17 and includes the signal sequence.

EGFRvIII is the most commonly occurring variant of the EGFR in human cancers (Kuan et al. Endocr Relat Cancer. 8(2):83-96 (2001)). During the process of gene amplification, a 267 amino acid deletion occurs in the extracellular domain of EGFR with a glycine residue inserted at the fusion junction. Thus, EGFRvIII lacks amino acids 6-273 of the extracellular domain of wild type EGFR and includes a glycine residue insertion at the junction. The EGFRvIII variant of EGFR contains a deletion of 267 amino acid residues in the extracellular domain where a glycine is inserted at the deletion junction. The EGFRvIII amino acid sequence is shown below as SEQ ID NO: 18.

The terms “specific binding” or “specifically binding”, as used herein, in reference to the interaction of an antibody or an ADC with a second chemical species, mean that the interaction is dependent upon the presence of a particular structure (e.g., an antigenic determinant or epitope) on the chemical species; for example, an antibody recognizes and binds to a specific protein structure rather than to proteins generally. If an antibody or ADC is specific for epitope “A”, the presence of a molecule containing epitope A (or free, unlabeled A), in a reaction containing labeled “A” and the antibody, will reduce the amount of labeled A bound to the antibody or ADC.

The term “antibody” refers to an immunoglobulin molecule that specifically binds to an antigen and comprises two heavy (H) chains and two light (L) chains. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, 1-R2, CDR2, 1-R3, CDR3, FR4.

In certain embodiments, an antibody can comprise a heavy chain having 1-5 amino acid deletions at the carboxy end of the heavy chain.

An “isolated antibody”, as used herein, is intended to refer to an antibody that is substantially free of other antibodies having different antigenic specificities (e.g., an isolated antibody that specifically binds EGFR is substantially free of antibodies that specifically bind antigens other than EGFR). An isolated antibody that specifically binds EGFR may, however, have cross-reactivity to other antigens, such as EGFR molecules from other species. Moreover, an isolated antibody may be substantially free of other cellular material and/or chemicals.

The term “epitope” refers to a region of an antigen that is bound by an antibody or ADC. In embodiments, epitope determinants include chemically active surface groupings of molecules such as amino acids, sugar side chains, phosphoryl, or sulfonyl, and, in embodiments, may have specific three dimensional structural characteristics, and/or specific charge characteristics. In embodiments, an antibody is said to specifically bind an antigen when it preferentially recognizes its target antigen in a complex mixture of proteins and/or macromolecules. In embodiments, the antibodies of the invention bind to an epitope defined by the amino acid sequence CGADSYEMEEDGVRKC (SEQ ID NO: 20) (which corresponds to amino acid residues 287-302 of the mature form of hEGFR).

The term “surface plasmon resonance”, as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, NJ). For further descriptions, see Jönsson, U., et al. (1993) Ann. Biol. Clin. 51:19-26; Jönsson, U., et al. (1991) Biotechniques 11:620-627; Johnsson, B., et al. (1995) J. Mol. Recognit. 8:125-131; and Johnnson, B., et al. (1991) Anal. Biochem. 198:268-277.

The term “kₐ”, as used herein, is intended to refer to the on rate constant for association of an antibody to the antigen to form the antibody/antigen complex.

The term “k_{d}”, as used herein, is intended to refer to the off rate constant for dissociation of an antibody from the antibody/antigen complex.

The term “K_{D}”, as used herein, is intended to refer to the equilibrium dissociation constant of a particular antibody-antigen interaction (e.g., AM2 antibody and EGFR). K_{D} is calculated by kₐ/k_{d}.

The term “antibody-drug-conjugate” or “ADC” refers to a binding protein, such as an antibody or antigen binding fragment thereof, chemically linked to one or more chemical drug(s) (also referred to herein as agent(s), warhead(s), or payload(s)) that may optionally be therapeutic or cytotoxic agents. In embodiments, an ADC includes an antibody, a cytotoxic or therapeutic drug, and a linker that enables attachment or conjugation of the drug to the antibody. Here, the ADC comprises an anti-EGFR antibody conjugated via a linker to a Bcl-xL inhibitor.

The terms “anti-Epidermal Growth Factor antibody drug conjugate,” “anti-EGFR antibody drug conjugate,” or “anti-EGFR ADC”, used interchangeably herein, refer to an ADC comprising an antibody that specifically binds to EGFR, whereby the antibody is conjugated to one or more chemical agent(s). Here, an anti-EGFR ADC comprises antibody AM2 conjugated to a Bcl-xL inhibitor.

### Anti-EGFR Antibodies And Antibody Drug Conjugates

The anti-EGFR antibodies described herein provide the ADCs of the present disclosure with the ability to bind to EGFR such that the cytotoxic Bcl-xL drug attached to the antibody may be delivered to the EGFR-expressing cell.

In embodiments, the present disclosure provides an anti-EGFR IgG1 antibody. In embodiments, the humanized IgG1 anti-EGFR antibody is AM2. The AM2 antibody comprises a heavy chain variable region comprising a CDR1 domain comprising the amino acid sequence set forth as SEQ ID NO: 2, a CDR2 domain comprising the amino acid sequence set forth as SEQ ID NO: 3, and a CDR3 domain comprising the amino acid sequence set forth as SEQ ID NO: 4, and a light chain variable region comprising a CDR1 domain comprising the amino acid sequence set forth as SEQ ID NO: 6, a CDR2 domain comprising the amino acid sequence set forth as SEQ ID NO: 7, and a CDR3 domain comprising the amino acid sequence set forth as SEQ ID NO: 8. The AM2 antibody comprises a heavy chain variable region comprising the amino acid sequence set forth as SEQ ID NO: 22 and a light chain variable region comprising the amino acid sequence set forth as SEQ ID NO: 23. The AM2 antibody comprises a heavy chain comprising the amino acid sequence set forth as SEQ ID NO: 1 and a light chain comprising the amino acid sequence set forth as SEQ ID NO: 5.

The AM2 antibody binds a truncated form of the EGFR extracellular domain (ECD) that mimics the conformation of active EGFR with a higher affinity than depatuxizumab (used as the antibody component of depatuxizumab mafodotin, ABT-414). Despite this significantly increased affinity for the active form of EGFR, AM2 lacks any measurable binding to the full-length wild-type EGFR ECD. The AM2 antibody maintains the binding characteristics of the AM1 antibody (losatuxizumab, used as the antibody component of losatuxizumab vedotin, ABBV-221), but has a distinct sequence in order to mitigate potential safety issues, such as the infusion reactions observed with ABBV-221. The AM2 antibody is in the z,a allotype, as opposed to the z, non-a allotype for losatuxizumab. The AM2 antibody incorporates a LALA mutation, which reduces interaction with Fcγ receptors. AM2 also incorporates a C6v1 (LC:C214A) mutation, enabling site-specific conjugation and controlled DAR. Taken together, AM2's novel sequence mitigates potential safety issues, including those seen with previous anti-EGFR ADCs.

Antibodies may be produced by any of a number of techniques. For example, expression from host cells, wherein expression vector(s) encoding the heavy and light chains is (are) transfected into a host cell by standard techniques.

In embodiments, this disclosure provides an anti-hEGFR ADC comprising an anti-hEGFR antibody conjugated to a synthon, wherein the synthon is 6-{8-[(1,3-benzothiazol-2-yl)carbamoyl]-3,4-dihydroisoquinolin-2(1H)-yl}-3-[1-({3-[2-({[(2-{2-[(2S,3R,4R,5S,6S)-6-S)-6-carboxy-3,4,5-trihydroxyoxan-2-yl]ethyl}-4-{[(2S)-2-{[(2S)-2-(2-{(3S,5S)-3-(2,5-dioxo-2,5-dehydro-1H-pyrrol-1-yl)-2-oxo-5-[(2- sulfoethoxy)methyl]pyrrolidin-1-yl}acetamido)-3-methylbutanoyl]amino}propanoyl]amino}phenyl)methoxy]carbonyl}[(3S)-3,4-dihydroxybutyl]amino)ethoxy]-5,7-dimethyladamantan-1-yl}methyl)-5-methyl-1H-pyrazol-4-yl]pyridine-2-carboxylic acid (“AAA”):

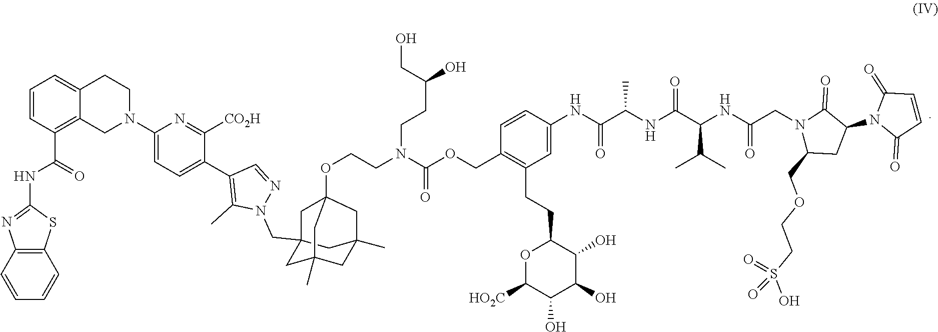 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CC[C@H](O)CO)C(=O)OCc1ccc(NC(=O)[C@H](C)NC(=O)[C@@H](NC(=O)CN4C(=O)[C@@H](N5C(=O)C=CC5=O)C[C@H]4COCCS(=O)(=O)O)C(C)C)cc1CC[C@@H]1O[C@H](C(=O)O)[C@@H](O)[C@H](O)[C@H]1O)(C3)C2]

In embodiments, the present disclosure provides an anti-human epidermal growth factor receptor (EGFR) antibody-drug conjugate comprising the following structure:

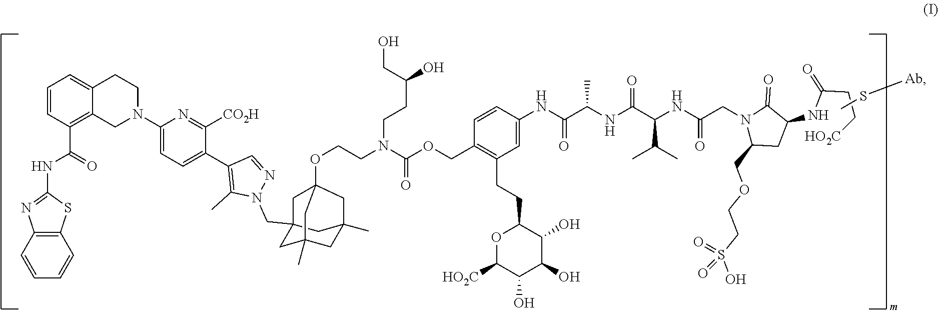

wherein m is an integer and Ab is an IgG1 anti-hEGFR antibody. In embodiments, the antibody comprises a heavy chain variable region comprising a heavy chain CDR1 domain comprising the amino acid sequence set forth as SEQ ID NO: 2, a heavy chain CDR2 domain comprising the amino acid sequence set forth as SEQ ID NO: 3, and a heavy chain CDR3 domain comprising the amino acid sequence set forth as SEQ ID NO: 4; and a light chain variable region comprising a light chain CDR1 domain comprising the amino acid sequence set forth as SEQ ID NO: 6, a light chain CDR2 domain comprising the amino acid sequence set forth as SEQ ID NO: 7, and a light chain CDR3 domain comprising the amino acid sequence set forth as SEQ ID NO: 8. In embodiments, the antibody Ab comprises a heavy chain variable region comprising the amino acid sequence set forth as SEQ ID NO: 22 and a light chain variable region comprising the amino acid sequence set forth as SEQ ID NO: 23. In embodiments, the antibody Ab comprises a heavy chain comprising the amino acid sequence set forth as SEQ ID NO: 1 and a light chain comprising the amino acid sequence set forth as SEQ ID NO: 5. In embodiments, m is an integer between 1 and 3. In embodiments, m is 2. In embodiments, the antibody is conjugated to the structure of formula (I) through C220 of the heavy chain (i.e., C219 of SEQ ID NO: 1).

In embodiments, the present disclosure provides an anti-human epidermal growth factor receptor (EGFR) antibody-drug conjugate comprising the following structure:

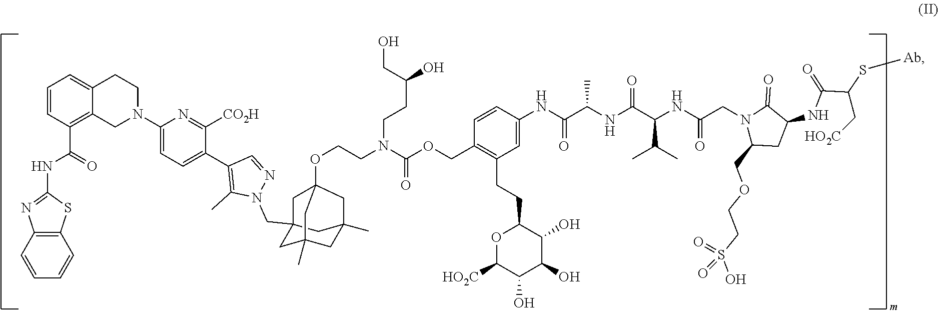

wherein m is an integer and Ab is an IgG1 anti-hEGFR antibody. In embodiments, the antibody comprises a heavy chain variable region comprising a heavy chain CDR1 domain comprising the amino acid sequence set forth as SEQ ID NO: 2, a heavy chain CDR2 domain comprising the amino acid sequence set forth as SEQ ID NO: 3, and a heavy chain CDR3 domain comprising the amino acid sequence set forth as SEQ ID NO: 4; and a light chain variable region comprising a light chain CDR1 domain comprising the amino acid sequence set forth as SEQ ID NO: 6, a light chain CDR2 domain comprising the amino acid sequence set forth as SEQ ID NO: 7, and a light chain CDR3 domain comprising the amino acid sequence set forth as SEQ ID NO: 8. In embodiments, the antibody Ab comprises a heavy chain variable region comprising the amino acid sequence set forth as SEQ ID NO: 22 and a light chain variable region comprising the amino acid sequence set forth as SEQ ID NO: 23. In embodiments, the antibody Ab comprises a heavy chain comprising the amino acid sequence set forth as SEQ ID NO: 1 and a light chain comprising the amino acid sequence set forth as SEQ ID NO: 5. In embodiments, m is an integer between 1 and 3. In embodiments, m is 2. In embodiments, the antibody is conjugated to the structure of formula (II) through C220 of the heavy chain (i.e., C219 of SEQ ID NO: 1).

In embodiments, the present disclosure provides an anti-human epidermal growth factor receptor (EGFR) antibody-drug conjugate comprising the following structure:

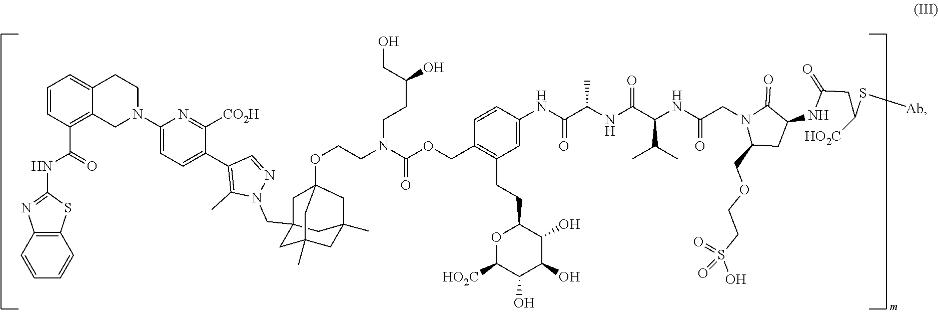

wherein m is an integer and Ab is an IgG1 anti-hEGFR antibody. In embodiments, the antibody comprises a heavy chain variable region comprising a heavy chain CDR1 domain comprising the amino acid sequence set forth as SEQ ID NO: 2, a heavy chain CDR2 domain comprising the amino acid sequence set forth as SEQ ID NO: 3, and a heavy chain CDR3 domain comprising the amino acid sequence set forth as SEQ ID NO: 4; and a light chain variable region comprising a light chain CDR1 domain comprising the amino acid sequence set forth as SEQ ID NO: 6, a light chain CDR2 domain comprising the amino acid sequence set forth as SEQ ID NO: 7, and a light chain CDR3 domain comprising the amino acid sequence set forth as SEQ ID NO: 8. In embodiments, the antibody Ab comprises a heavy chain variable region comprising the amino acid sequence set forth as SEQ ID NO: 22 and a light chain variable region comprising the amino acid sequence set forth as SEQ ID NO: 23. In embodiments, the antibody Ab comprises a heavy chain comprising the amino acid sequence set forth as SEQ ID NO: 1 and a light chain comprising the amino acid sequence set forth as SEQ ID NO: 5. In embodiments, m is an integer between 1 and 3. In embodiments, m is 2. In embodiments, the antibody is conjugated to the structure of formula (III) through C220 of the heavy chain (i.e., C219 of SEQ ID NO: 1).

In embodiments, the antibodies and antibody-drug conjugates of the present disclosure, such as the AM2 antibody and AM2-AAA ADC, bind to EGFR(1-525) (SEQ ID NO: 16) with a dissociation constant (K_{D}) of 1×10⁻⁶M or less, such as between 1×10⁻⁶M and about 1×10⁻¹⁰M, or between about 1×10⁻⁶M and about 1×10⁻⁷M, as determined by surface plasmon resonance.

In embodiments, the antibodies of the present disclosure, such as the AM2 antibody and AM2-AAA ADC, bind to EGFRvIII (SEQ ID NO: 18) with a K_{D} of about 6×10⁻⁹M or less, or about 5.5×10⁻⁹M or less, or 5.0×10⁻⁹M or less, as determined by surface plasmon resonance.

### Methods of Use

One embodiment pertains to a method of treating non-small cell lung cancer, comprising administering to a subject having non-small cell lung cancer an anti-EGFR ADC as described herein, in an amount effective to provide therapeutic benefit.

### EXAMPLES

The following examples provide synthetic methods for Bcl-X_{L} inhibitor (Example 1.1.17) and synthon (AAA, Example 1). Methods of synthesizing bcl-xL inhibitors and synthons such as AAA may be found, for example, in U.S. Patent Application Publication No. 2019/0343961 (AbbVie, Inc.), which is incorporated by reference herein in its entirety.

The examples were named using ACD/Name 2012 release (Build 56084, 05 April 2012, Advanced Chemistry Development Inc., Toronto, Ontario), ACD/Name 2014 release (Build 66687, 25 October 2013, Advanced Chemistry Development Inc., Toronto, Ontario), ACD/Name 2019.1.1 release (Build 110555, 18 July 2019, Advanced Chemistry Development Inc., Toronto, Ontario), ChemDraw® Ver. 9.0.7 (CambridgeSoft, Cambridge, MA), ChemDraw® Ultra Ver. 12.0 (CambridgeSoft, Cambridge, MA), or ChemDraw ® Professional Ver. 15.0.0.106. Bcl-XL inhibitor and synthon intermediates were named with ACD/Name 2012 release (Build 56084, 05 April 2012, Advanced Chemistry Development Inc., Toronto, Ontario), ACD/Name 2014 release (Build 66687, 25 October 2013, Advanced Chemistry Development Inc., Toronto, Ontario), ACD/Name 2019.1.1 release (Build 110555, 18 July 2019, Advanced Chemistry Development Inc., Toronto, Ontario), ChemDraw® Ver. 9.0.7 (CambridgeSoft, Cambridge, MA), ChemDraw® Ultra Ver. 12.0 (CambridgeSoft, Cambridge, MA), or ChemDraw® Professional Ver. 15.0.0.106.

Abbreviations that may be used herein are:

| Abbreviation | Definition | Abbreviation | Definition |
|---|---|---|---|
| FACS | fluorescent activated cell | HIC | hydrophobic interaction |
| | sorting | | chromatography |
| HEPES | N-2-hydroxyethylpiperazine- | HPLC | high performance liquid |
| | N-ethanesulfonic acid buffer | | chromatography |
| DAR | drug to antibody ratio | MS | mass spectrometry |
| DMA | dimethyl acetamide | PBS | phosphate buffer saline |
| DMSO | dimethyl sulfoxide | NSCLC | non-small-cell lung |
| | | | carcinoma |
| FBS | fetal bovine serum | SEC | size exclusion |
| | | | chromatography |
| TCEP | (tris(2- | q | quartet |
| | carboxyethyl)phosphine) | | |
| mL | milliliter | min | minute |
| NMR | nuclear magnetic resonance | mL | milliliter |
| LC/MS or | liquid chromatography- mass | μL | microliter |
| LCMS or | spectrometry | | |
| LC-MS | | | |
| s | singlet | L | liter |
| br s | broad singlet | g | gram |
| d | duplet or doublet | mg | milligram |
| m | multiplet | mmol | millimoles |
| t | triplet | dd | doublet of doublets |
| td | triplet of doublets | br t | broad triplet |
| br d | broad doublet | mM | millimolar |
| br m | broad multiplet | ppm | parts per million |
| M | molarity (moles/liter) | DCI | desorption chemical |
| | | | ionization |
| N | normality (equivalent/liter) | w/w | weight for weight |
| APCI | atmospheric pressure | mm | millimeter |
| | chemical ionization | | |
| ESI | electrospray ionization | MHz | megahertz |
| m/z | mass divided by charge | μm | micrometer |
| | number | | |
| psi | pounds per square inch | BIM | Bcl-2-like protein 11 |
| v/v | volume for volume | DPBS | Dulbecco's phosphate- |
| | | | buffered saline |
| SCID | Severe combined | | |
| | immunodeficiency | | |
| TMP | transmembrane pressure | | |

### Example 1

6-{8-[(1,3-benzothiazol-2-yl)carbamoyl]-3,4-dihydroisoquinolin-2(1H)-yl}-3-[1-({3-[2-({[(2-{2-[(2S,3R,4R,5S,6S)-6-S)-6-carboxy-3,4,5-trihydroxyoxan-2-yl]ethyl}-4-{[(2S)-2-{[(2S)-2-(2-{(3S,5S)-3-(2,5-dioxo-2,5-dehydro-1H-pyrrol-1-yl)-2-oxo-5-[(2-sulfoethoxy)methyl]pyrrolidin-1-yl}acetamido)-3-methylbutanoyl]amino}propanoyl]amino}phenyl)methoxy]carbonyl}[(3S)-3,4-dihydroxybutyl]amino)ethoxy]-5,7-dimethyladamantan-1-yl}methyl)-5- methyl-1H-pyrazol-4-yl]pyridine-2-carboxylic acid (Synthon AAA):

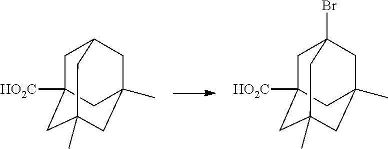 [SMILES: CC12CC3CC(C)(C1)CC(C(=O)O)(C3)C2 ; CC12CC3(C)CC(Br)(C1)CC(C(=O)O)(C2)C3]

### Example 1.1.1

### 3-bromo-5,7-dimethyladamantane-1-carboxylic Acid

Into a 50 mL round-bottomed flask at 0° C., was added bromine (16 mL). Iron powder (7 g) was added, and the reaction mixture was stirred at 0° C. for 30 minutes. 3,5-Dimethyladamantane-1-carboxylic acid (12 g) was added. The mixture was warmed up to room temperature and stirred for 3 days. A mixture of ice and concentrated HCl was poured into the reaction mixture. The resulting suspension was treated twice with Na₂SO₃ (50 g in 200 mL water) and extracted three times with dichloromethane. The combined organic fractions were washed with 1 N aqueous HCl, dried over sodium sulfate, filtered, and concentrated to give the title compound.

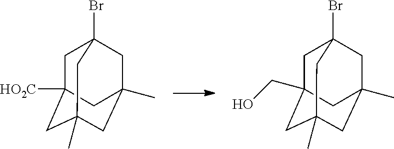 [SMILES: CC12CC3(C)CC(Br)(C1)CC(C(=O)O)(C2)C3 ; CC12CC3(C)CC(Br)(C1)CC(CO)(C2)C3]

### Example 1.1.2

### (3-bromo-5,7-dimethyladamantane-1-yl)Methanol

To a solution of Example 1.1.1 (15.4 g) in tetrahydrofuran (200 mL) was added BH₃ (1 M in tetrahydrofuran, 150 mL), and the mixture was stirred at room temperature overnight. The reaction mixture was then carefully quenched by adding methanol dropwise. The mixture was then concentrated under vacuum, and the residue was partitioned between ethyl acetate (500 mL) and 2 N aqueous HCl (100 mL). The aqueous layer was further extracted twice with ethyl acetate, and the combined organic extracts were washed with water and brine, dried over sodium sulfate, and filtered. The filtrate was concentrated to give the title compound.

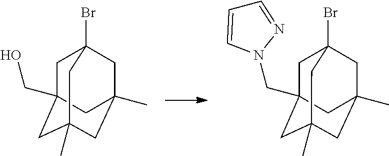 [SMILES: CC12CC3(C)CC(Br)(C1)CC(CO)(C2)C3 ; CC12CC3(C)CC(Br)(C1)CC(Cn1cccn1)(C2)C3]

### Example 1.1.3

### 1-[(3-bromo-5,7-dimethyladamantan-1-yl)methyl]-1H-pyrazole

To a solution of Example 1.1.2 (8.0 g) in toluene (60 mL) was added 1H-pyrazole (1.55 g) and cyanomethylenetributylphosphorane (2.0 g), and the mixture was stirred at 90° C. overnight. The reaction mixture was concentrated, and the residue was purified by silica gel column chromatography (10:1 heptane:ethyl acetate) to give the title compound. MS (ESI) m/z 324.2 (M+H)⁺.

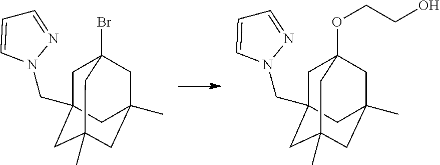 [SMILES: CC12CC3(C)CC(Br)(C1)CC(Cn1cccn1)(C2)C3 ; CC12CC3(C)CC(Cn4cccn4)(C1)CC(OCCO)(C2)C3]

### Example 1.1.4

### 2-({3,5-dimethyl-7-[(1H-pyrazol-1-yl)methyl]adamantan-1-yl}oxy)ethan-1-ol

To a solution of Example 1.1.3 (4.0 g) in ethane-1,2-diol (12 mL) was added triethylamine (3 mL). The mixture was stirred at 150° C. under microwave conditions (Biotage® Initiator) for 45 minutes. The mixture was poured into water (100 mL) and extracted three times with ethyl acetate. The combined organic extracts were washed with water and brine, dried over sodium sulfate, and filtered. Concentration of the filtrate gave a residue that was purified by silica gel chromatography, eluted with 20% ethyl acetate in heptane, followed by 5% methanol in dichloromethane, to give the title compound. MS (ESI) m/z 305.2 (M+H)⁺.

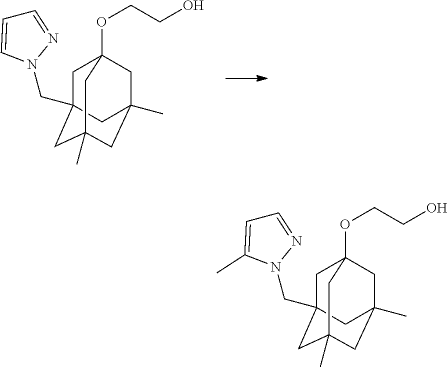 [SMILES: CC12CC3(C)CC(Cn4cccn4)(C1)CC(OCCO)(C2)C3 ; Cc1ccnn1CC12CC3(C)CC(C)(C1)CC(OCCO)(C3)C2]

### Example 1.1.5

### 2-({3,5-dimethyl-7-[(5-methyl-1H-pyrazol-1-yl)methyl]adamantan-1-yl}oxy)ethan-1-ol

To a cooled (−78° C.) solution of Example 1.1.4 (6.05 g) in tetrahydrofuran (100 mL) was added n-butyllithium (40 mL, 2.5 M in hexane), and the mixture was stirred at −78° C. for 1.5 hours. Iodomethane (10 mL) was added through a syringe, and the mixture was stirred at −78° C. for 3 hours. The reaction mixture was then quenched with aqueous NH₄Cl and extracted twice with ethyl acetate, and the combined organic extracts were washed with water and brine. After drying over sodium sulfate, the solution was filtered and concentrated, and the residue was purified by silica gel column chromatography, eluted with 5% methanol in dichloromethane, to give the title compound. MS (ESI) m/z 319.5 (M+H)⁺.

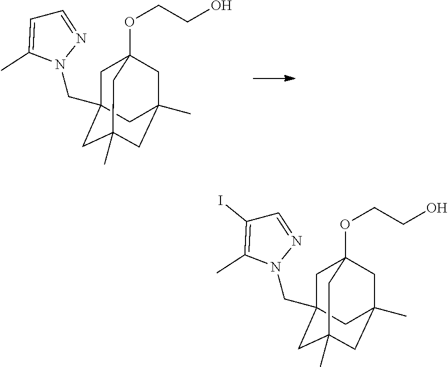 [SMILES: Cc1ccnn1CC12CC3(C)CC(C)(C1)CC(OCCO)(C3)C2 ; Cc1c(I)cnn1CC12CC3(C)CC(C)(C1)CC(OCCO)(C3)C2]

### Example 1.1.6

### 2-({3-[(4-iodo-5-methyl-1H-pyrazol-1-yl)methyl]-5,7-dimethyladamantan-1-yl}oxy)ethan-1-ol

To a solution of Example 1.1.5 (3.5 g) in N,N-dimethylformamide (30 mL) was added N-iodosuccinimide (3.2 g), and the mixture was stirred at room temperature for 1.5 hours. The reaction mixture was diluted with ethyl acetate (600 mL) and washed with aqueous 10% w/w NaHSO₃, water and brine. The organic layer was dried over sodium sulfate, filtered, and concentrated under reduced pressure. The residue was purified by silica gel chromatography, eluted with 20% ethyl acetate in dichloromethane, to give the title compound. MS (ESI) m/z 445.3 (M+H)⁺.

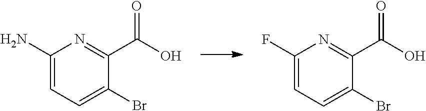 [SMILES: Nc1ccc(Br)c(C(=O)O)n1 ; O=C(O)c1nc(F)ccc1Br]

### Example 1.1.7

### 3-bromo-6-fluoropyridine-2-carboxylic Acid

A slurry of 6-amino-3-bromopyridine-2-carboxylic acid (25 g) in 1:1 dichloromethane/chloroform (400 mL) was added to nitrosonium tetrafluoroborate (18.2 g) in dichloromethane (100 mL) at 5° C. over 1 hour. The resulting mixture was stirred for another 30 minutes, then warmed to 35° C. and stirred overnight. The reaction was cooled to room temperature, and then adjusted to pH 4 with aqueous NaH₂PO₄ solution. The resulting solution was extracted three times with dichloromethane, and the combined extracts were washed with brine, dried over sodium sulfate, filtered, and concentrated to provide the title compound.

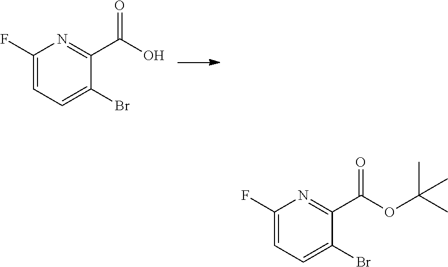

### Example 1.1.8

### tert-butyl 3-bromo-6-fluoropyridine-2-carboxylate

para-Toluenesulfonyl chloride (27.6 g) was added to a solution of Example 1.1.7 (14.5 g) and pyridine (26.7 mL) in dichloromethane (100 mL) and tert-butanol (80 mL) at 0° C. The reaction was stirred for 15 minutes, and then warmed to room temperature, and stirred overnight. The solution was concentrated and partitioned between ethyl acetate and saturated aqueous Na₂CO₃ solution. The layers were separated, and the aqueous layer extracted with ethyl acetate. The organic layers were combined, rinsed with aqueous Na₂CO₃ solution and brine, dried over sodium sulfate, filtered, and concentrated to provide the title compound.

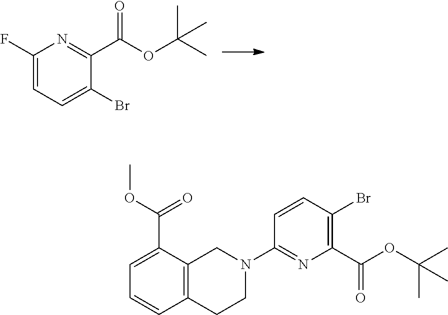 [SMILES: CC(C)(C)OC(=O)c1nc(F)ccc1Br ; COC(=O)c1cccc2c1CN(c1ccc(Br)c(C(=O)OC(C)(C)C)n1)CC2]

### Example 1.1.9

### methyl 2-[5-bromo-6-(tert-butoxycarbonyl)pyridin-2-yl]-1,2,3,4-tetrahydroisoquinoline-8-carboxylate

To a solution of methyl 1,2,3,4-tetrahydroisoquinoline-8-carboxylate hydrochloride (12.37 g) and Example 1.1.8 (15 g) in dimethyl sulfoxide (100 mL) was added N,N-diisopropylethylamine (12 mL), and the mixture was stirred at 50° C. for 24 hours. The mixture was then diluted with ethyl acetate (500 mL) and washed with water and brine. The organic layer was dried over sodium sulfate, filtered, and concentrated under reduced pressure. The residue was purified by silica gel chromatography, eluted with 20% ethyl acetate in hexane, to give the title compound. MS (ESI) m/z 448.4 (M+H)⁺.

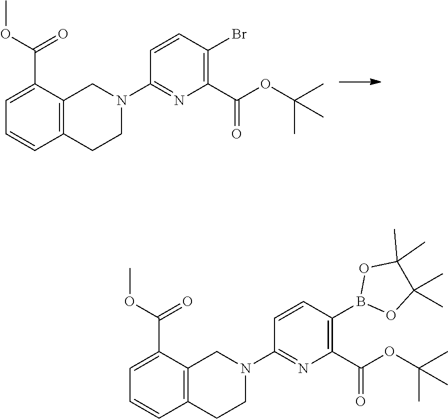 [SMILES: COC(=O)c1cccc2c1CN(c1ccc(Br)c(C(=O)OC(C)(C)C)n1)CC2 ; COC(=O)c1cccc2c1CN(c1ccc(B3OC(C)(C)C(C)(C)O3)c(C(=O)OC(C)(C)C)n1)CC2]

### Example 1.1.10

### methyl 2-[6-(tert-butoxycarbonyl)-5-(4,4,5,5-tetramethyl-1,3,2-dioxaborolan-2-yl)pyridin-2-yl]-1,2,3,4-tetrahydroisoquinoline-8- carboxylate

To a solution of Example 1.1.9 (2.25 g) and [1,1′-bis(diphenylphosphino)ferrocene]dichloropalladium(II) (205 mg) in acetonitrile (30 mL) was added triethylamine (3 mL) and pinacolborane (2 mL), and the mixture was stirred at reflux for 3 hours. The mixture was diluted with ethyl acetate (200 mL) and washed with water and brine. The organic layer was dried over sodium sulfate, filtered, and concentrated under reduced pressure. Purification of the residue by silica gel chromatography, eluted with 20% ethyl acetate in hexane, provided the title compound.

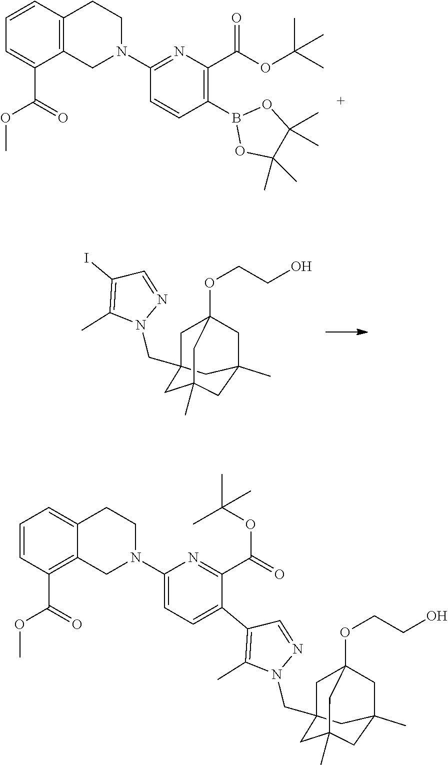

### Example 1.1.11

### methyl 2-[6-(tert-butoxycarbonyl)-5-(1-{[3-(2-hydroxyethoxy)-5,7-dimethyladamantan-1-yl]methyl}-5-methyl-1H-pyrazol-4-yl)pyridin-2-yl]-1,2,3,4-tetrahydroisoquinoline-8-carboxylate

To a solution of Example 1.1.10 (2.25 g) in tetrahydrofuran (30 mL) and water (10 mL) was added Example 1.1.6 (2.0 g), 1,3,5,7-tetramethyl-6-phenyl-2,4,8-trioxa-6-phosphaadamantane (329 mg), tris(dibenzylideneacetone)dipalladium(0) (206 mg) and potassium phosphate tribasic (4.78 g). The mixture was refluxed overnight, cooled, and diluted with ethyl acetate (500 mL). The resulting mixture was washed with water and brine, and the organic layer was dried over sodium sulfate, filtered, and concentrated. The residue was purified by flash chromatography on silica gel, eluted with 20% ethyl acetate in heptanes followed by 5% methanol in dichloromethane, to provide the title compound.

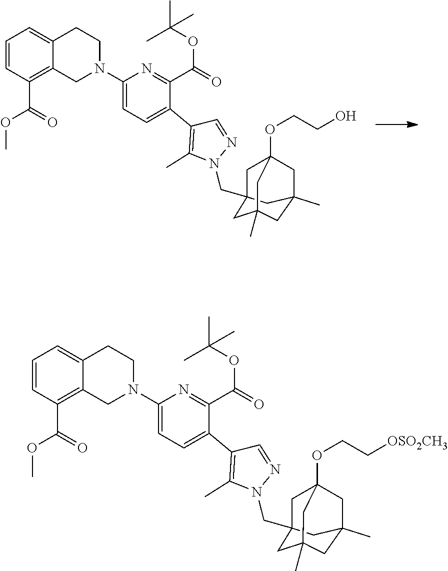

### Example 1.1.12

### methyl 2-[6-(tert-butoxycarbonyl)-5-{1-[(3-[(methanesulfonyl)oxy]ethoxyl-5,7-dimethyladamantan-1-yl)methyl]-5-methyl-1H- pyrazol-4-yl}pyridin-2-yl]-1,2,3,4-tetrahydroisoquinoline-8-carboxylate

To a cold solution of Example 1.1.11 (3.32 g) in dichloromethane (100 mL) in an ice-bath was sequentially added triethylamine (3 mL) and methanesulfonyl chloride (1.1 g). The reaction mixture was stirred at room temperature for 1.5 hours, diluted with ethyl acetate, and washed with water and brine. The organic layer was dried over sodium sulfate, filtered, and concentrated to provide the title compound.

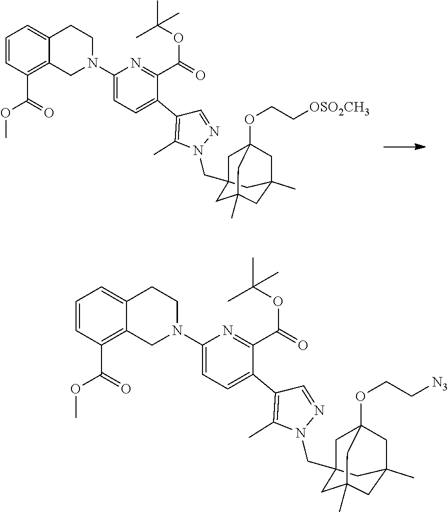

### Example 1.1.13

### methyl 2-[5-(1-1[3-(2-azidoethoxy)-5,7-dimethyladamantan-1-yl]methyll-5-methyl-1H-pyrazol-4-yl)-6-(tert- butoxycarbonyl)pyridin-2-yl]-1,2,3,4-tetrahydroisoquinoline-8-carboxylate

To a solution of Example 1.1.12 (16.5 g) in N,N-dimethylformamide (120 mL) was added sodium azide (4.22 g). The mixture was heated at 80° C. for 3 hours, cooled, diluted with ethyl acetate, and washed with water and brine. The organic layer was dried over sodium sulfate, filtered, and concentrated. The residue was purified by flash chromatography on silica gel, eluted with 20% ethyl acetate in heptanes, to provide the title compound.

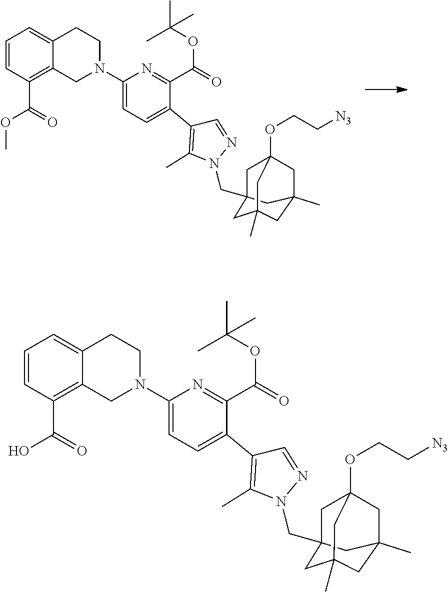 [SMILES: COC(=O)c1cccc2c1CN(c1ccc(-c3cnn(CC45CC6(C)CC(C)(C4)CC(OCCN=[N+]=[N-])(C6)C5)c3C)c(C(=O)OC(C)(C)C)n1)CC2 ; Cc1c(-c2ccc(N3CCc4cccc(C(=O)O)c4C3)nc2C(=O)OC(C)(C)C)cnn1CC12CC3(C)CC(C)(C1)CC(OCCN=[N+]=[N-])(C3)C2]

### Example 1.1.14

### 2-[5-(1-{[3-(2-azidoethoxy)-5,7-dimethyladamantan-1-yl]methyl}-5-methyl-1H-pyrazol-4-yl)-6-(tert- butoxycarbonyl)pyridin-2-yl]-1,2,3,4-tetrahydroisoquinoline-8-carboxylic Acid

To a solution of Example 1.1.13 (10 g) in a mixture of tetrahydrofuran (60 mL), methanol (30 mL) and water (30 mL) was added lithium hydroxide monohydrate (1.2 g). The mixture was stirred at room temperature overnight and neutralized with 2% aqueous HCl. The resulting mixture was concentrated, and the residue was dissolved in ethyl acetate (800 mL) and washed with brine. The organic layer was dried over sodium sulfate, filtered, and concentrated to provide the title compound.

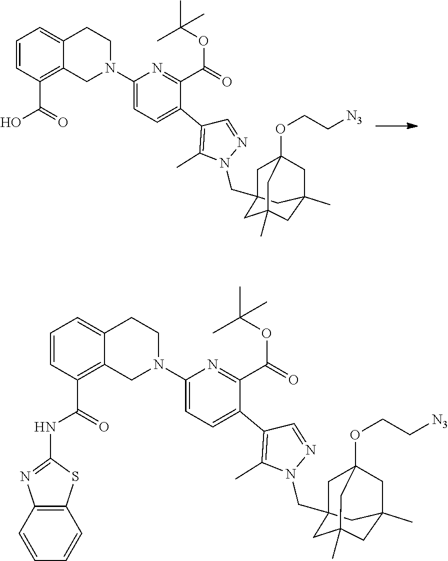 [SMILES: Cc1c(-c2ccc(N3CCc4cccc(C(=O)O)c4C3)nc2C(=O)OC(C)(C)C)cnn1CC12CC3(C)CC(C)(C1)CC(OCCN=[N+]=[N-])(C3)C2 ; Cc1c(-c2ccc(N3CCc4cccc(C(=O)Nc5nc6ccccc6s5)c4C3)nc2C(=O)OC(C)(C)C)cnn1CC12CC3(C)CC(C)(C1)CC(OCCN=[N+]=[N-])(C3)C2]

### Example 1.1.15

### tert-butyl 3-(1-{[3-(2-azidoethoxy)-5,7-dimethyladamantan-1-yl]methyll-5-methyl-1H-pyrazol-4-yl)-6-18-[(1,3- benzothiazol-2-yl)carbamoyl]-3,4-dihydroisoquinolin-2(1H)-yl}pyridine-2-carboxylate

A mixture of Example 1.1.14 (10 g), benzo[d]thiazol-2-amine (3.24 g), fluoro-N,N,N′,N′-tetramethylformamidinium hexafluorophosphate (5.69 g) and N,N-diisopropylethylamine (5.57 g) in N,N -dimethylformamide (20 mL) was heated at 60° C. for 3 hours, cooled and diluted with ethyl acetate. The resulting mixture was washed with water and brine. The organic layer was dried over sodium sulfate, filtered, and concentrated. The residue was purified by flash chromatography on silica gel, eluted with 20% ethyl acetate in dichloromethane to give the title compound.

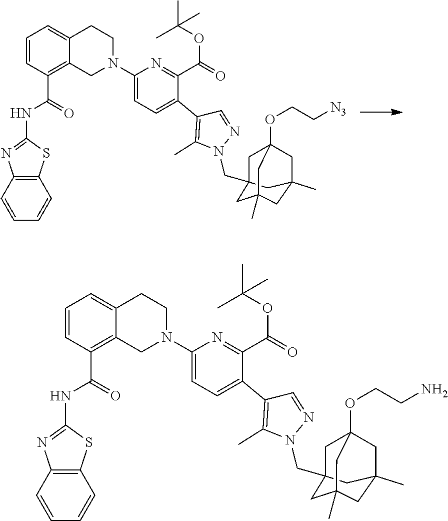 [SMILES: Cc1c(-c2ccc(N3CCc4cccc(C(=O)Nc5nc6ccccc6s5)c4C3)nc2C(=O)OC(C)(C)C)cnn1CC12CC3(C)CC(C)(C1)CC(OCCN=[N+]=[N-])(C3)C2 ; Cc1c(-c2ccc(N3CCc4cccc(C(=O)Nc5nc6ccccc6s5)c4C3)nc2C(=O)OC(C)(C)C)cnn1CC12CC3(C)CC(C)(C1)CC(OCCN)(C3)C2]

### Example 1.1.16

### tert-butyl 3-(1-{[3-(2-aminoethoxy)-5,7-dimethyladamantan-1-yl]methyll-5-methyl-1H-pyrazol-4-yl)-6-18-[(1,3- benzothiazol-2-yl)carbamoyl]-3,4-dihydroisoquinolin-2(1H)-yl}pyridine-2-carboxylate

To a solution of Example 1.1.15 (2.0 g) in tetrahydrofuran (30 mL) was added Pd/C (10%, 200 mg). The mixture was stirred under a hydrogen atmosphere (18 psi) overnight. The insoluble material was filtered off and the filtrate was concentrated to provide the title compound.

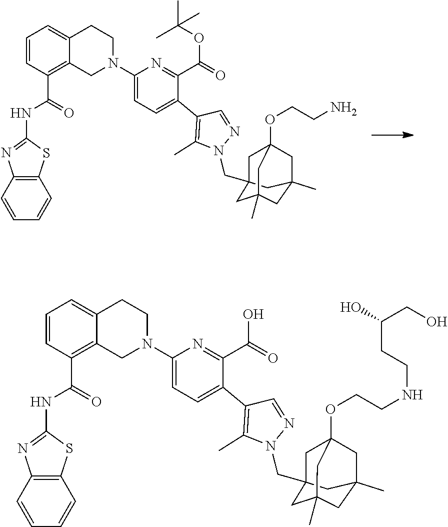 [SMILES: Cc1c(-c2ccc(N3CCc4cccc(C(=O)Nc5nc6ccccc6s5)c4C3)nc2C(=O)OC(C)(C)C)cnn1CC12CC3(C)CC(C)(C1)CC(OCCN)(C3)C2 ; Cc1c(-c2ccc(N3CCc4cccc(C(=O)Nc5nc6ccccc6s5)c4C3)nc2C(=O)O)cnn1CC12CC3(C)CC(C)(C1)CC(OCCNCC[C@H](O)CO)(C3)C2]

### Example 1.1.17

### 6-{8-[(1,3-benzothiazol-2-yl)carbamoyl]-3,4-dihydroisoquinolin-2(1H)-yl}-3-(1-{[3-(2-{[(3S)-3,4-dihydroxybutyl]amino}ethoxy)-5,7-dimethyladamantan-1-yl]methyl}-5-methyl-1H-pyrazol-4-yl)pyridine-2-carboxylic Acid

To a solution of Example 1.1.16 (213 mg) in dichloromethane (2 mL) was added (S)-2-(2,2-dimethyl-1,3-dioxolan-4-yl)acetaldehyde (42 mg). After stirring at room temperature for 30 minutes, sodium triacetoxyborohydride (144 mg) was added. The reaction mixture was stirred at room temperature overnight. Trifluoroacetic acid (2 mL) was added and stirring was continued overnight. The reaction mixture was concentrated, and the residue was purified by reverse-phase HPLC using a Gilson system (Phenomenex® Luna® C18 250×50 mm column), eluted with 5-85% acetonitrile in water containing 0.1% v/v trifluoroacetic acid (100 mL/minute). The desired fractions were combined and freeze-dried to provide the title compound. ¹H NMR (400 MHz, dimethyl sulfoxide-d₆) δ ppm 12.86 (s, 1H), 8.22 (d, 2H), 8.05 — 8.01 (m, 1H), 7.79 (d, 1H), 7.61 (d, 1H), 7.53 — 7.41 (m, 3H), 7.36 (td, 2H), 7.28 (s, 1H), 6.95 (d, 1H), 4.95 (s, 2H), 3.88 (t, 2H), 3.82 (s, 2H), 3.26 — 2.94 (m, 7H), 2.10 (s, 3H), 1.84 — 1.75 (m, 1H), 1.52-1.63 (m, 1H), 1.45 — 1.23 (m, 6H), 1.19 — 0.96 (m, 7H), 0.86 (s, 6H); MS (ESI) m/z 834.3 (M+H)⁺.

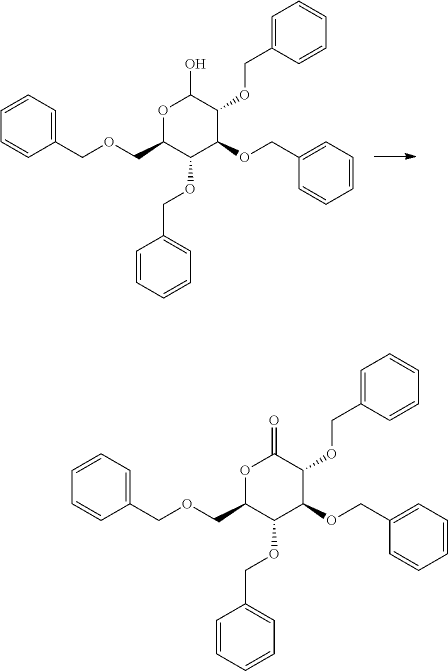 [SMILES: OC1O[C@H](COCc2ccccc2)[C@@H](OCc2ccccc2)[C@H](OCc2ccccc2)[C@H]1OCc1ccccc1 ; O=C1O[C@H](COCc2ccccc2)[C@@H](OCc2ccccc2)[C@H](OCc2ccccc2)[C@H]1OCc1ccccc1]

### Example 1.2.1

### (3R,4S,5R,6R)-3,4,5-tris(benzyloxy)-6-[(benzyloxy)methyl]oxan-2-one

To a mixture of (3R,4S,5R,6R)-3,4,5-tris(benzyloxy)-6-[(benzyloxy)methyl]oxan-2-ol (75 g) in dimethyl sulfoxide (400 mL) at 0° C. was added acetic anhydride (225 mL). The mixture was stirred for 16 hours at room temperature before it was cooled to 0° C. A large volume of water was added and stirring was stopped allowing the reaction mixture to settle for 3 hours (the crude lactone migrated to the bottom of the flask). The supernatant was removed, and the crude mixture was diluted with ethyl acetate and was washed 3 times with water, neutralized with a saturated aqueous mixture of NaHCO₃, and washed again twice with water. The organic layer was then dried over magnesium sulfate, filtered, and concentrated to give the title compound. MS (ESI) m/z 561 (M+Na)⁺.

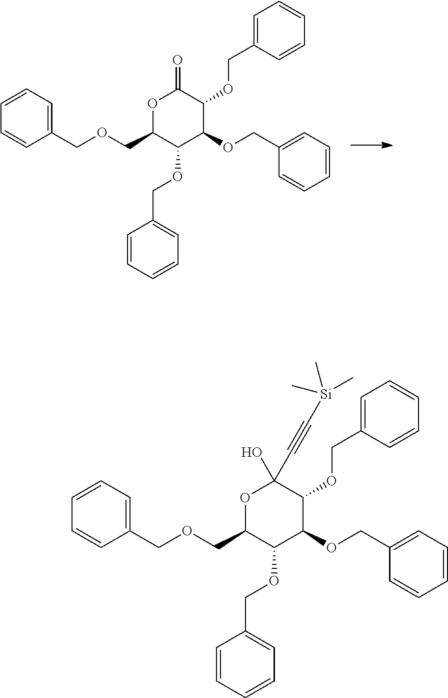 [SMILES: O=C1O[C@H](COCc2ccccc2)[C@@H](OCc2ccccc2)[C@H](OCc2ccccc2)[C@H]1OCc1ccccc1 ; C[Si](C)(C)C#CC1(O)O[C@H](COCc2ccccc2)[C@@H](OCc2ccccc2)[C@H](OCc2ccccc2)[C@H]1OCc1ccccc1]

### Example 1.2.2

### (3R,4S,5R,6R)-3,4,5-tris(benzyloxy)-6-((benzyloxy)methyl)-2-((trimethylsilyl)ethynyl)tetrahydro-2H-pyran-2-ol

To a mixture of ethynyltrimethylsilane (18.23 g) in tetrahydrofuran (400 mL) under nitrogen and chilled in a dry ice/acetone bath (internal temp −65° C.) was added 2.5 M butyllithium in hexane (55.7 mL) dropwise, keeping the temperature below −60° C. The mixture was stirred in a cold bath for 40 minutes, followed by an ice-water bath (internal temperature rose to 0.4° C.) for 40 minutes, and finally cooled to −75° C. again. A mixture of Example 1.2.1 (50 g) in tetrahydrofuran (50 mL) was added dropwise, keeping the internal temperature below −70° C. The mixture was stirred in a dry ice/acetone bath for an additional 3 hours. The reaction was quenched with saturated aqueous NaHCO₃ (250 mL). The mixture was allowed to warm to room temperature, extracted with ethyl acetate (3×300 mL), dried over MgSO₄, filtered, and concentrated in vacuo to give the title compound. MS (ESI) m/z 659 (M+Na)⁺.

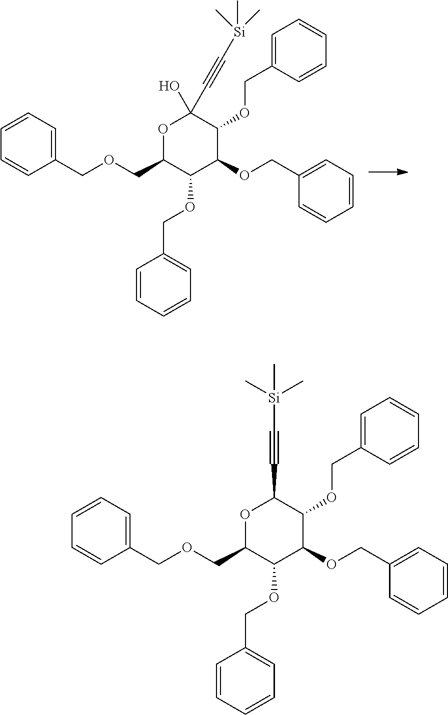 [SMILES: C[Si](C)(C)C#CC1(O)O[C@H](COCc2ccccc2)[C@@H](OCc2ccccc2)[C@H](OCc2ccccc2)[C@H]1OCc1ccccc1 ; C[Si](C)(C)C#C[C@@H]1O[C@H](COCc2ccccc2)[C@@H](OCc2ccccc2)[C@H](OCc2ccccc2)[C@H]1OCc1ccccc1]

### Example 1.2.3

### trimethyl({(2S,3S,4R,5R,6R)-3,4,5-tris(benzyloxy)-6-[(benzyloxy)methyl]oxan-2-yl}ethynyl)silane

To a mixed mixture of Example 1.2.2 (60 g) in acetonitrile (450 mL) and dichloromethane (150 mL) at −15° C. in an ice-salt bath was added triethylsilane (81 mL) dropwise, followed by addition of boron trifluoride diethyl ether complex (40.6 mL) at such a rate that the internal temperature did not exceed −10° C. The mixture was then stirred at −15° C. to −10° C. for 2 hours. The reaction was quenched with saturated aqueous NaHCO₃ (275 mL) and stirred for 1 hour at room temperature. The mixture was then extracted with ethyl acetate (3×550 mL). The combined extracts were dried over MgSO₄, filtered, and concentrated. The residue was purified by flash chromatography eluted with a gradient of 0% to 7% ethyl acetate/petroleum ether to give the title compound. MS (ESI) m/z 643 (M+Na)⁺.

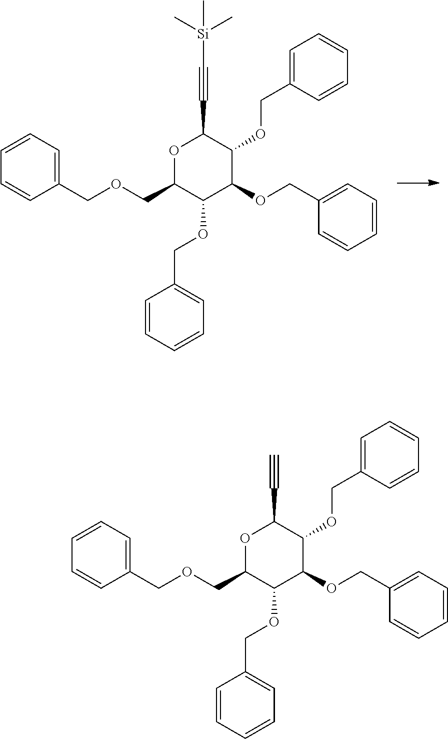 [SMILES: C[Si](C)(C)C#C[C@@H]1O[C@H](COCc2ccccc2)[C@@H](OCc2ccccc2)[C@H](OCc2ccccc2)[C@H]1OCc1ccccc1 ; C#C[C@@H]1O[C@H](COCc2ccccc2)[C@@H](OCc2ccccc2)[C@H](OCc2ccccc2)[C@H]1OCc1ccccc1]

### Example 1.2.4

### (2R,3R,4R,5S,6S)-3,4,5-tris(benzyloxy)-2-[(benzyloxy)methyl]-6-ethynyloxane

To a mixture of Example 1.2.3 (80 g) in dichloromethane (200 mL) and methanol (1000 mL) was added 1 N aqueous NaOH (258 mL). The mixture was stirred at room temperature for 2 hours and then concentrated. The residue was then partitioned between water and dichloromethane. The combined organic extracts were washed with brine, dried over Na₂SO₄, filtered, and concentrated to give the title compound. MS (ESI) m/z 571 (M+Na)⁺.

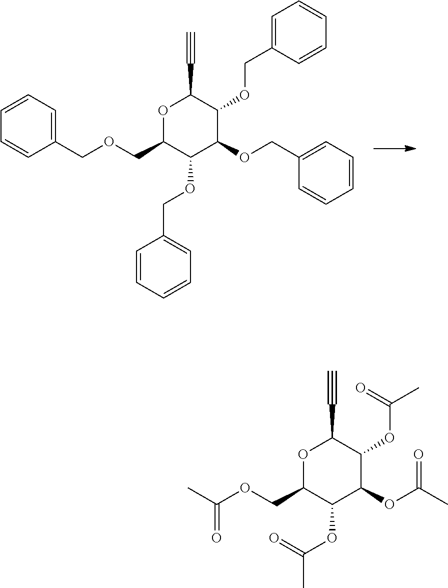 [SMILES: C#C[C@@H]1O[C@H](COCc2ccccc2)[C@@H](OCc2ccccc2)[C@H](OCc2ccccc2)[C@H]1OCc1ccccc1 ; C#C[C@@H]1O[C@H](COC(C)=O)[C@@H](OC(C)=O)[C@H](OC(C)=O)[C@H]1OC(C)=O]

### Example 1.2.5

### (2R,3R,4R,5S,6S)-2-[(acetyloxy)methyl]-6-ethynyloxane-3,4,5-triyl triacetate

To a mixture of Example 1.2.4 (66 g) in acetic anhydride (500 mL) cooled by an ice/water bath was added boron trifluoride diethyl ether complex (152 mL) dropwise. The mixture was stirred at room temperature for 16 hours, cooled with an ice/water bath and neutralized with saturated aqueous NaHCO₃. The mixture was extracted with ethyl acetate (3×500 mL), dried over Na₂SO₄, filtered, and concentrated in vacuo. The residue was purified by flash chromatography eluted with a gradient of 0% to 30% ethyl acetate/petroleum ether to give the title compound. MS (ESI) m/z 357 (M+H)⁺.

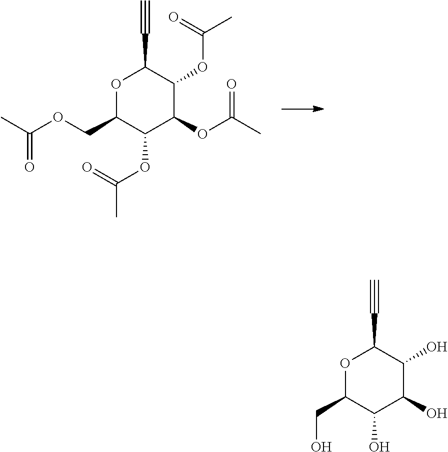 [SMILES: C#C[C@@H]1O[C@H](COC(C)=O)[C@@H](OC(C)=O)[C@H](OC(C)=O)[C@H]1OC(C)=O ; C#C[C@@H]1O[C@H](CO)[C@@H](O)[C@H](O)[C@H]1O]

### Example 1.2.6

### (2S,3R,4R,5S,6R)-2-ethynyl-6-(hydroxymethyl)oxane-3,4,5-triol

To a mixture of Example 1.2.5 (25 g) in methanol (440 mL) was added sodium methanolate (2.1 g). The mixture was stirred at room temperature for 2 hours, and then neutralized with 4 M HCl in dioxane. The mixture was concentrated, and the residue was adsorbed onto silica gel and loaded onto a silica gel column. The column was eluted with a gradient of 0 to 100% ethyl acetate/petroleum ether then 0% to 12% methanol/ethyl acetate to give the title compound. MS (ESI) m/z 211 (M+Na)⁺.

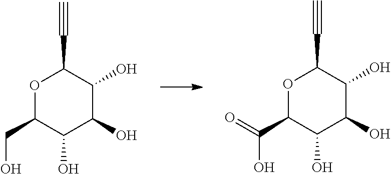

### Example 1.2.7

### (2S,3S,4R,5R,6S)-6-ethynyl-3,4,5-trihydroxyoxane-2-carboxylic Acid

A three-necked round bottom flask was charged with Example 1.2.6 (6.00 g), KBr (0.30 g), tetrabutylammonium bromide (0.41 g) and 60 mL of saturated aqueous NaHCO₃. TEMPO ((2.2,6,6-tetramethylpiperidin-1-yl)oxyl, 0.15 g) in dichloromethane (60 mL) was added. The mixture was stirred vigorously and cooled in an ice-salt bath to −2° C. internal temperature. A mixture of brine (12 mL), saturated aqueous NaHCO₃ (24 mL) and 10 weight % aqueous NaOC1 (154 mL) solution was added dropwise such that the internal temperature was maintained below 2° C. The pH of the reaction mixture was maintained in the 8.2-8.4 range with the addition of solid Na₂CO₃. After a total of 6 hours, the reaction mixture was cooled to 3° C. internal temperature and ethanol (˜20 mL) was added dropwise. The mixture was stirred for ˜30 minutes. The mixture was transferred to a separatory funnel, and the dichloromethane layer was discarded. The pH of the aqueous layer was adjusted to 2-3 using 1 M aqueous HCl. The aqueous layer was then concentrated to dryness to afford a solid. Methanol (100 mL was) added to the dry solid, and the slurry was stirred for ˜30 minutes. The mixture was filtered over a pad of diatomaceous earth, and the residue in the funnel was washed with ˜100 mL of methanol. The filtrate was concentrated under reduced pressure to obtain the title compound.

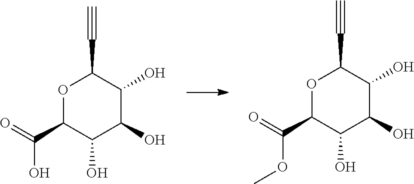

### Example 1.2.8

### methyl (2S,3S,4R,5R,6S)-6-ethynyl-3,4,5-trihydroxyoxane-2-carboxylate

A 500 mL three-necked round bottom flask was charged with a suspension of Example 1.2.7 (6.45 g) in methanol (96 mL), and the mixture was cooled in an ice-salt-bath with internal temperature of −1° C. Neat thionyl chloride (2.79 mL) was carefully added. The internal temperature kept rising throughout the addition but did not exceed 10° C. The reaction was allowed to slowly warm up to 15-20° C. over 2.5 hours. After 2.5 hours, the reaction was concentrated to give the title compound.

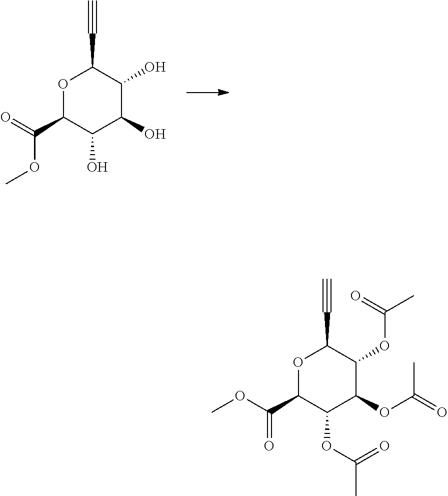 [SMILES: C#C[C@@H]1O[C@H](C(=O)OC)[C@@H](O)[C@H](O)[C@H]1O ; C#C[C@@H]1O[C@H](C(=O)OC)[C@@H](OC(C)=O)[C@H](OC(C)=O)[C@H]1OC(C)=O]

### Example 1.2.9

### methyl (2S,3S,4R,5S,6S)-3,4,5-tris(acetyloxy)-6-ethynyloxane-2-carboxylate

To Example 1.2.8 (6.9 g) as a mixture in N,N-dimethylformamide (75 mL) was added 4-(dimethylamino)pyridine (0.17 g) and acetic anhydride (36.1 mL). The suspension was cooled in an ice-bath and pyridine (18.04 mL) was added via syringe over 15 minutes. The reaction was allowed to warm to room temperature overnight. Additional acetic anhydride (12 mL) and pyridine (6 mL) were added and stirring was continued for an additional 6 hours. The reaction was cooled in an ice-bath and 250 mL of saturated aqueous NaHCO3 mixture was added and stirred for 1 hour. Water (100 mL) was added, and the mixture was extracted with ethyl acetate. The organic extract was washed twice with saturated CuSO₄ mixture, dried, filtered, and concentrated. The residue was purified by flash chromatography, eluted with 50% ethyl acetate/petroleum ether to give the title compound. ¹H NMR (500 MHz, methanol-d₄) δ ppm 5.29 (t, 1H), 5.08 (td, 2H), 4.48 (dd, 1H), 4.23 (d, 1H), 3.71 (s, 3H), 3.04 (d, 1H), 2.03 (s, 3H), 1.99 (s, 3H), 1.98 (s, 4H).

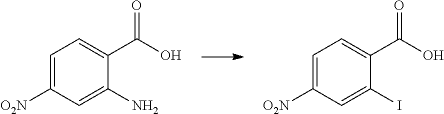 [SMILES: Nc1cc([N+](=O)[O-])ccc1C(=O)O ; O=C(O)c1ccc([N+](=O)[O-])cc1I]

### Example 1.2.10

### 2-iodo-4-nitrobenzoic Acid

A 3-L fully jacketed flask equipped with a mechanical stirrer, temperature probe and an addition funnel under a nitrogen atmosphere, was charged with 2-amino-4-nitrobenzoic acid (69.1 g, Combi-Blocks) and sulfuric acid, 1.5 M aqueous (696 mL). The resulting suspension was cooled to 0° C. internal temperature, and a mixture of sodium nitrite (28.8 g) in water (250 mL) was added dropwise over 43 minutes with the temperature kept below 1° C. The reaction was stirred at ca. 0° C. for 1 hour. A mixture of potassium iodide (107 g) in water (250 mL) was added dropwise over 44 minutes with the internal temperature kept below 1° C. (Initially addition was exothermic and there was gas evolution). The reaction was stirred 1 hour at 0° C. The temperature was raised to 20° C. and then stirred at ambient temperature overnight. The reaction mixture became a suspension. The reaction mixture was filtered, and the collected solid was washed with water. The wet solid (˜108 g) was stirred in 10% sodium sulfite (350 mL, with ˜200 mL water used to wash in the solid) for 30 minutes. The suspension was acidified with concentrated hydrochloric acid (35 mL), and the solid was collected by filtration and washed with water. The solid was slurried in water (1 L) and re-filtered, and the solid was left to dry in the funnel overnight. The solid was then dried in a vacuum oven for 2 hours at 60° C. The resulting solid was triturated with dichloromethane (500 mL), and the suspension was filtered and washed with additional dichloromethane. The solid was air-dried to give the title compound.

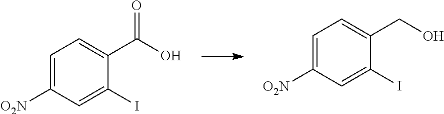 [SMILES: O=C(O)c1ccc([N+](=O)[O-])cc1I ; O=[N+]([O-])c1ccc(CO)c(I)c1]

### Example 1.2.11

### (2-iodo-4-nitrophenyl)Methanol

A flame-dried 3-L 3-necked flask was charged with Example 1.2.10 (51.9 g) and tetrahydrofuran (700 mL). The mixture was cooled in an ice bath to 0.5° C., and borane-tetrahydrofuran complex (443 mL, 1 M in tetrahydrofuran) was added dropwise (gas evolution) over 50 minutes, reaching a final internal temperature of 1.3° C. The reaction mixture was stirred for 15 minutes, and the ice bath was removed. The reaction was left to come to ambient temperature over 30 minutes. A heating mantle was installed, and the reaction was heated to an internal temperature of 65.5° C. for 3 hours, and then allowed to cool to room temperature while stirring overnight. The reaction mixture was cooled in an ice bath to 0° C. and quenched by dropwise addition of methanol (400 mL). After a brief incubation period, the temperature rose quickly to 2.5° C. with gas evolution. After the first 100 mL are added over ˜30 minutes, the addition was no longer exothermic, and the gas evolution ceased. The ice bath was removed, and the mixture was stirred at ambient temperature under nitrogen overnight. The mixture was concentrated to a solid, dissolved in dichloromethane/methanol and adsorbed on to silica gel (˜150 g). The residue was loaded on a plug of silica gel (3000 mL) and eluted with dichloromethane to give the title compound.

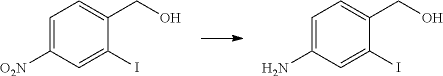 [SMILES: O=[N+]([O-])c1ccc(CO)c(I)c1 ; Nc1ccc(CO)c(I)c1]

### Example 1.2.12

### (4-amino-2-iodophenyl)Methanol

A 5-L flask equipped with a mechanical stirrer, heating mantle controlled by a JKEM temperature probe and a condenser was charged with Example 1.2.11 (98.83 g) and ethanol (2 L). The reaction was stirred rapidly, and iron (99 g) was added, followed by a mixture of ammonium chloride (20.84 g) in water (500 mL). The reaction was heated over the course of 20 minutes to an internal temperature of 80.3° C., where it began to reflux vigorously. The mantle was dropped until the reflux calmed. Thereafter, the mixture was heated to 80° C. for 1.5 hours. The reaction was filtered hot through a membrane filter, and the iron residue was washed with hot 50% ethyl acetate/methanol (800 mL). The eluent was passed through a diatomaceous earth pad, and the filtrate was concentrated. The residue was partitioned between 50% brine (1500 mL) and ethyl acetate (1500 mL). The layers were separated, and the aqueous layer was extracted with ethyl acetate (400 mL×3). The combined organic layers were dried over sodium sulfate, filtered, and concentrated to give the title compound, which was used without further purification.

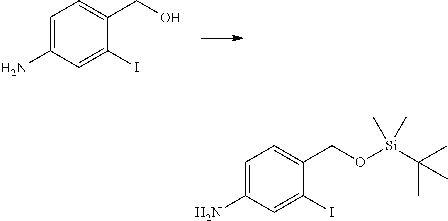 [SMILES: Nc1ccc(CO)c(I)c1 ; CC(C)(C)[Si](C)(C)OCc1ccc(N)cc1I]

### Example 1.2.13

### 4-({[tert-butyl(dimethyl)silyl]oxy}methyl)-3-iodoaniline

A 5-L flask with a mechanical stirrer was charged with Example 1.2.12 (88 g) and dichloromethane (2 L). The suspension was cooled in an ice bath to an internal temperature of 2.5° C., and tert-butylchlorodimethylsilane (53.3 g) was added portion-wise over 8 minutes. After 10 minutes, 1H-imidazole (33.7 g) was added portion wise to the cold reaction. The reaction was stirred for 90 minutes while the internal temperature rose to 15° C. The reaction mixture was diluted with water (3 L) and dichloromethane (1 L). The layers were separated, and the organic layer was dried over sodium sulfate, filtered, and concentrated to an oil. The residue was purified by silica gel chromatography (1600 g silica gel), eluted with a gradient of 0-25% ethyl acetate in heptane, to give the title compound.

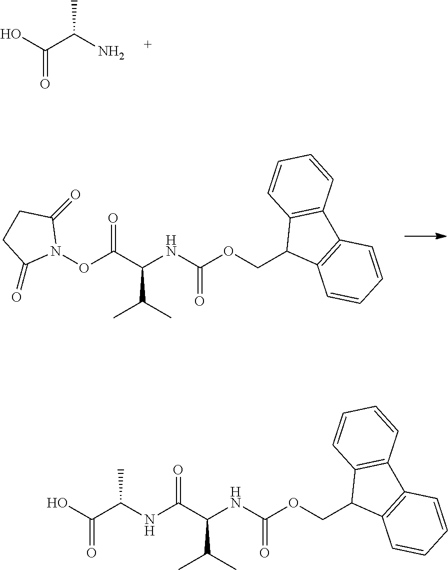

### Example 1.2.14

### (2S)-2-{[(2S)-2-({[(9H-fluoren-9-yl)methoxy]carbonyl}amino)-3-methylbutanoyl]amino}propanoic Acid

To a mixture of (9H-fluoren-9-yl)methyl {(2RS)-1-[(2,5-dioxopyrrolidin-1-yl)oxy]-3-methyl-1-oxobutan-2-yl}carbamate (6.5 g) in dimethoxyethane (40 mL) was added (2S)-2-aminopropanoic acid (1.393 g) and sodium bicarbonate (1.314 g) in water (40 mL). Tetrahydrofuran (20 mL) was added to aid solubility. The resulting mixture was stirred at room temperature for 16 hours. Aqueous citric acid (15%, 75 mL) was added, and the mixture was extracted with 10% 2-propanol in ethyl acetate (2×100 mL). A precipitate formed in the organic layer. The combined organic layers were washed with water (2×150 mL). The organic layer was concentrated under reduced pressure and then triturated with diethyl ether (80 mL). After brief sonication, the title compound was collected by filtration and air-dried. MS (ESI) m/z 411 (M+H)⁺.

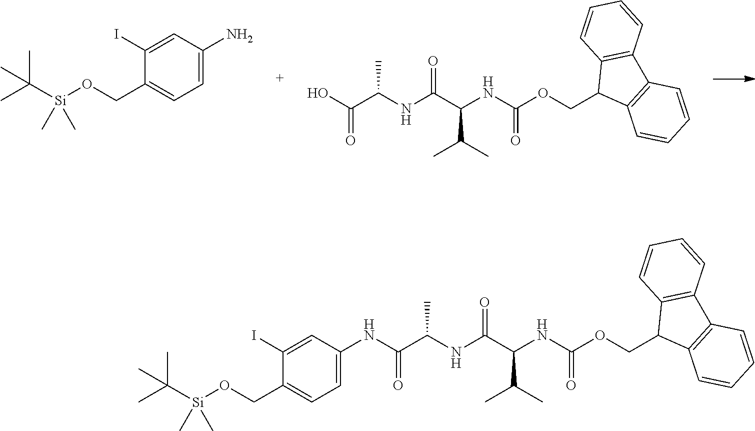 [SMILES: CC(C)(C)[Si](C)(C)OCc1ccc(N)cc1I ; CC(C)[C@H](NC(=O)OCC1c2ccccc2-c2ccccc21)C(=O)N[C@@H](C)C(=O)O ; CC(C)[C@H](NC(=O)OCC1c2ccccc2-c2ccccc21)C(=O)N[C@@H](C)C(=O)Nc1ccc(CO[Si](C)(C)C(C)(C)C)c(I)c1]

### Example 1.2.15

### (9H-fluoren-9-yl)methyl [R2S)-1-({(2S)-1-[4-({[tert-butyl(dimethyl)silyl]oxy}methyl)-3-iodoanilino]-1-oxopropan-2-yl}amino)-3-methyl-1-oxobutan-2-yl]carbamate

To a mixture of Example 1.2.13 (5.44 g) and Example 1.2.14 (6.15 g) in a mixture of dichloromethane (70 mL) and methanol (35.0 mL) was added ethyl 2-ethoxyquinoline-1(2H)-carboxylate (4.08 g), and the reaction mixture was stirred overnight. The reaction mixture was concentrated, and the residue was loaded onto silica gel, eluted with a gradient of 10% to 95% ethyl acetate in heptane followed by 5% methanol in dichloromethane. The product-containing fractions were concentrated, and the residue was dissolved in 0.2% methanol in dichloromethane (50 mL) and loaded onto silica gel eluted with a gradient of 0.2% to 2% methanol in dichloromethane. The product containing fractions were collected to give the title compound. MS (ESI) m/z 756.0 (M+H)⁺.

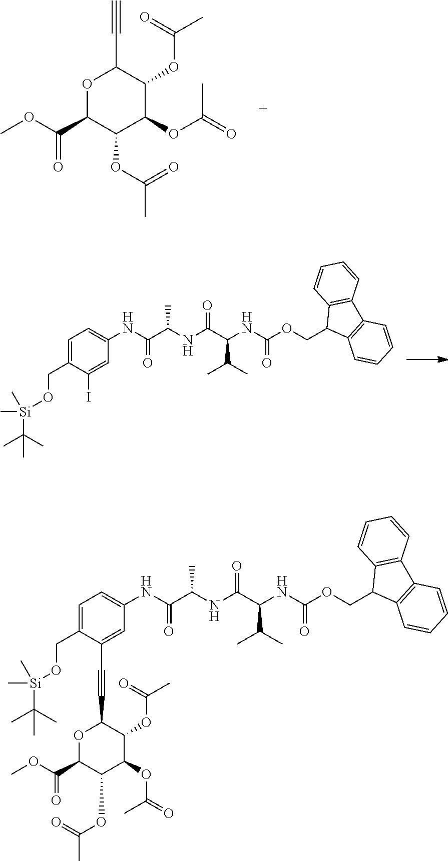 [SMILES: C#CC1O[C@H](C(=O)OC)[C@@H](OC(C)=O)[C@H](OC(C)=O)[C@H]1OC(C)=O ; CC(C)[C@H](NC(=O)OCC1c2ccccc2-c2ccccc21)C(=O)N[C@@H](C)C(=O)Nc1ccc(CO[Si](C)(C)C(C)(C)C)c(I)c1 ; COC(=O)[C@H]1O[C@@H](C#Cc2cc(NC(=O)[C@H](C)NC(=O)[C@@H](NC(=O)OCC3c4ccccc4-c4ccccc43)C(C)C)ccc2CO[Si](C)(C)C(C)(C)C)[C@H](OC(C)=O)[C@@H](OC(C)=O)[C@@H]1OC(C)=O]

### Example 1.2.16

### methyl (2S,3S,4R,5S,6S)-3,4,5-tris(acetyloxy)-6-{[2-({[tert-butyl(dimethyl)silyl]oxy}methyl)-5-{[(2S)-2- {[(2S)-2-({[(9H-fluoren-9-yl)methoxy]carbonyl}amino)-3-methylbutanoyl]amino}propanoyl]amino}phenyl]ethynyl}oxane-2-carboxylate

A mixture of Example 1.2.9 (4.500 g), Example 1.2.15 (6.62 g), copper(I) iodide (0.083 g) and bis(triphenylphosphine)palladium(II) dichloride (0.308 g) were combined in vial and degassed. N,N-Dimethylformamide (45 mL) and N-ethyl-N-(propan-2-yl)propan-2-amine (4.55 mL) were added, and the reaction vessel was flushed with nitrogen and stirred at room temperature overnight. The reaction was partitioned between water (100 mL) and ethyl acetate (250 mL). The layers were separated, and the organic layer was dried over magnesium sulfate, filtered, and concentrated. The residue was purified by silica gel chromatography, eluted with a gradient of 5% to 95% ethyl acetate in heptane. The product containing fractions were collected and concentrated. The residue was purified by silica gel chromatography, eluted with a gradient of 0.25% to 2.5% methanol in dichloromethane to give the title compound. MS (ESI) m/z 970.4 (M+H)⁺.

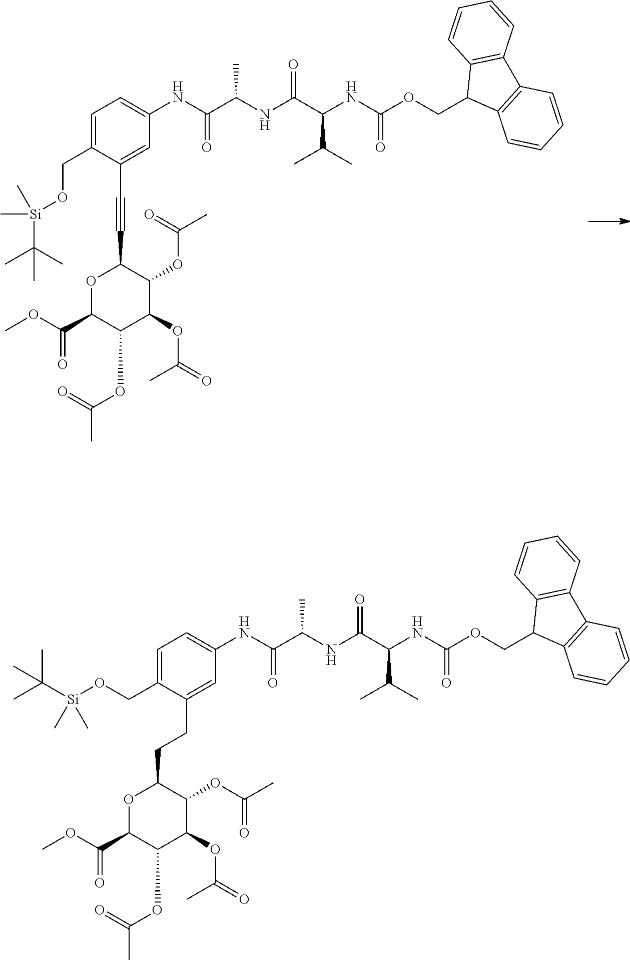 [SMILES: COC(=O)[C@H]1O[C@@H](C#Cc2cc(NC(=O)[C@H](C)NC(=O)[C@@H](NC(=O)OCC3c4ccccc4-c4ccccc43)C(C)C)ccc2CO[Si](C)(C)C(C)(C)C)[C@H](OC(C)=O)[C@@H](OC(C)=O)[C@@H]1OC(C)=O ; COC(=O)[C@H]1O[C@@H](CCc2cc(NC(=O)[C@H](C)NC(=O)[C@@H](NC(=O)OCC3c4ccccc4-c4ccccc43)C(C)C)ccc2CO[Si](C)(C)C(C)(C)C)[C@H](OC(C)=O)[C@@H](OC(C)=O)[C@@H]1OC(C)=O]

### Example 1.2.17

### methyl (2S,3S,4R,5S,6S)-3,4,5-tris(acetyloxy)-6-{2-[2-({[tert-butyl(dimethyl)silyl]oxy}methyl)-5-{[(2S)-2-{[(2S)-2-({[(9H-fluoren-9-yl)methoxy]carbonyl}amino)-3-methylbutanoyl]amino}propanoyl]amino}phenyl]ethyl}oxane-2-carboxylate

Example 1.2.16 (4.7 g) and tetrahydrofuran (95 mL) were added to 5% Pt/C (2.42 g, wet) in a 50 mL pressure bottle and shaken for 90 minutes at room temperature under 50 psi of hydrogen. The reaction mixture was filtered and concentrated to give the title compound. MS (ESI) m/z 974.6 (M+H)⁺.

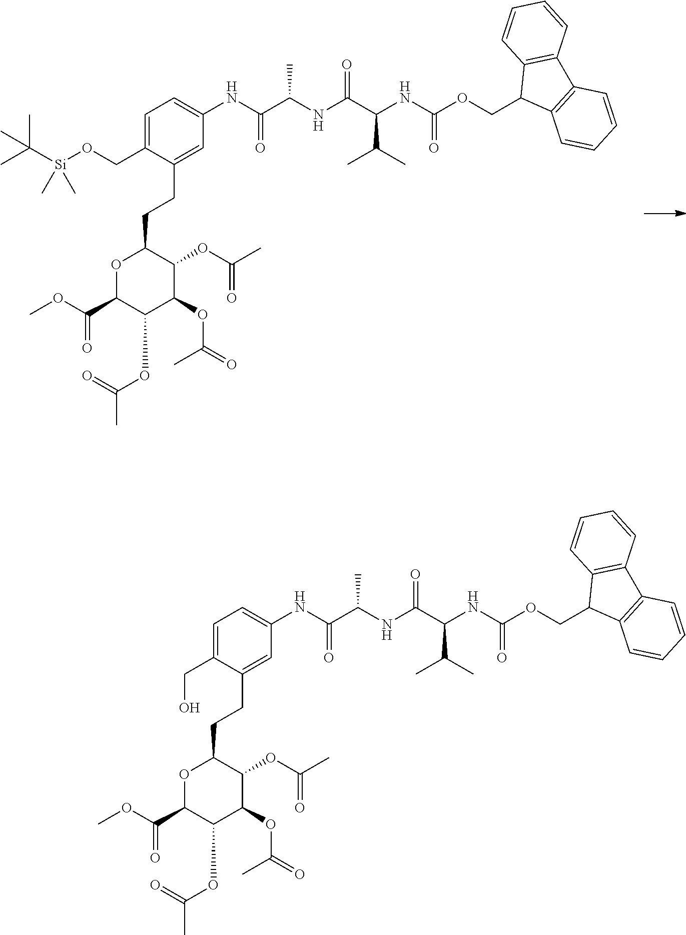 [SMILES: COC(=O)[C@H]1O[C@@H](CCc2cc(NC(=O)[C@H](C)NC(=O)[C@@H](NC(=O)OCC3c4ccccc4-c4ccccc43)C(C)C)ccc2CO[Si](C)(C)C(C)(C)C)[C@H](OC(C)=O)[C@@H](OC(C)=O)[C@@H]1OC(C)=O ; COC(=O)[C@H]1O[C@@H](CCc2cc(NC(=O)[C@H](C)NC(=O)[C@@H](NC(=O)OCC3c4ccccc4-c4ccccc43)C(C)C)ccc2CO)[C@H](OC(C)=O)[C@@H](OC(C)=O)[C@@H]1OC(C)=O]

### Example 1.2.18

### methyl (2S,3S,4R,5S,6S)-3,4,5-tris(acetyloxy)-6-{2-[5-{[(2S)-2-{[(2S)2-({[(9H-fluoren-9-yl)methoxy]carbonyl}amino)-3-methylbutanoyl]amino}propanoyl]amino}-2-(hydroxymethyl)phenyl]ethyl}oxane-2-carboxylate

A mixture of Example 1.2.17 (5.4 g) in tetrahydrofuran (7 mL), water (7 mL) and glacial acetic acid (21 mL) was stirred overnight at room temperature. The reaction mixture was diluted with ethyl acetate (200 mL) and washed with water (100 mL), saturated aqueous NaHCO₃ (100 mL), and brine (100 mL). The organic fraction was dried over magnesium sulfate, filtered, and concentrated. The residue was purified by silica gel chromatography, eluted with a gradient of 0.5% to 5% methanol in dichloromethane, to give the title compound. MS (ESI) m/z 860.4 (M+H)⁺.

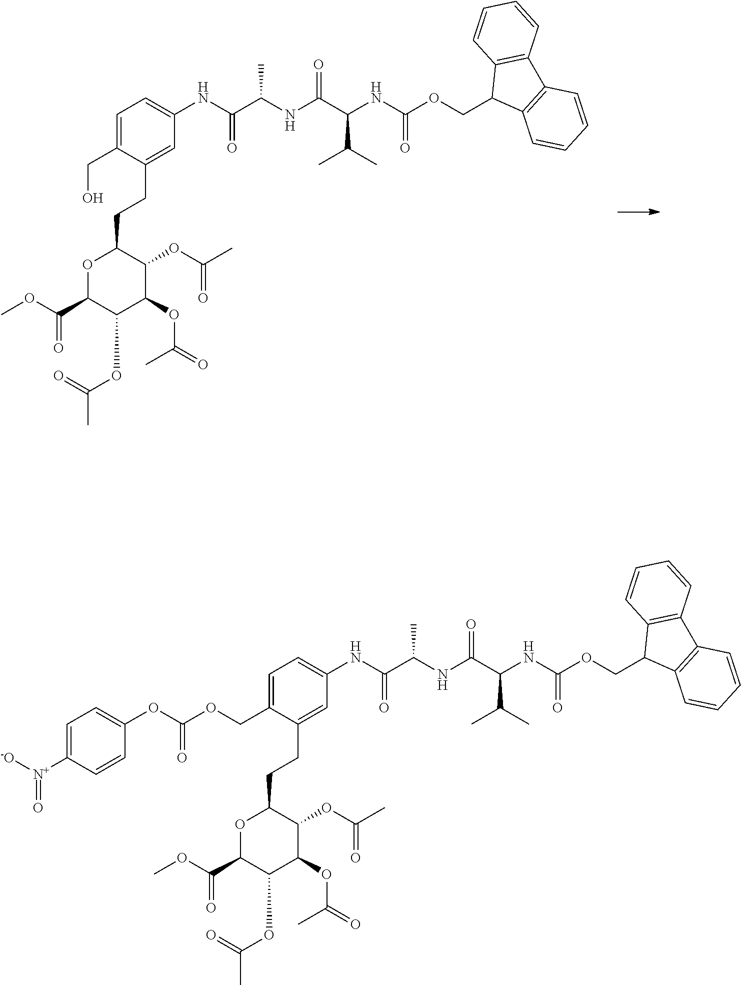

### Example 1.2.19

### methyl (2S,3S,4R,5S,6S)-3,4,5-tris(acetyloxy)-6-{2-[5-{[(2S)-2-{[(2S)-2-({[9H-fluoren-9-yl)methoxy]carbonyl}amino)-3-methylbutanoyl]amino}propanoyl]amino}-2-({[(4-nitrophenoxy)carbonyl]oxy}methyl)phenyl]ethyl}oxane-2- carboxylate

To a mixture of Example 1.2.18 (4.00 g) and bis(4-nitrophenyl) carbonate (2.83 g) in acetonitrile (80 mL) was added N-ethyl-N-(propan-2-yl)propan-2-amine (1.22 mL) at room temperature. After stirring overnight, the reaction was concentrated, dissolved in dichloromethane (250 mL) and washed with saturated aqueous NaHCO₃ mixture (4×150 mL). The organic layer was dried over magnesium sulfate, filtered, and concentrated. The resulting foam was purified by silica gel chromatography, eluted with a gradient of 5% to 75% ethyl acetate in hexanes to give the title compound. MS (ESI) m/z 1025.5 (M+H)⁺.

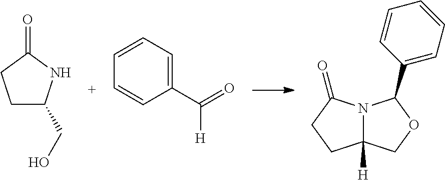 [SMILES: O=C1CC[C@@H](CO)N1 ; O=Cc1ccccc1 ; O=C1CC[C@H]2CO[C@H](c3ccccc3)N12]

### Example 1.3.1

### (3R,7aS)-3-phenyltetrahydro-3H,5H-pyrrolo[1,2-c][1,3]oxazol-5-one

A mixture of (5S)-5-(hydroxymethyl)pyrrolidin-2-one (25 g), benzaldehyde (25.5 g) and para-toluenesulfonic acid monohydrate (0.50 g) in toluene (300 mL) was heated to reflux using a Dean-Stark trap under a drying tube for 16 hours. The reaction mixture was cooled to room temperature, and the solvent was decanted from the insoluble materials. The decanted organic layer was washed with saturated aqueous sodium bicarbonate mixture (twice) and brine (once). The organic layer was dried over sodium sulfate, filtered, and concentrated under reduced pressure. The residue was purified by flash chromatography on silica gel, eluted with 35/65 heptane/ethyl acetate, to give the title compound. MS (DCI) m/z 204.0 (M+H)⁺.

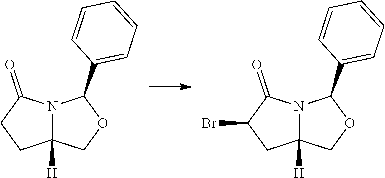 [SMILES: O=C1CC[C@H]2CO[C@H](c3ccccc3)N12 ; O=C1[C@H](Br)C[C@H]2CO[C@H](c3ccccc3)N12]

### Example 1.3.2

### (3R,6R,7aS)-6-bromo-3-phenyltetrahydro-3H,5H-pyrrolo[1,2-c][1,3]oxazol-5-one

To a cold (−77° C.) mixture of Example 1.3.1 (44.6 g) in tetrahydrofuran (670 mL) was added lithium bis(trimethylsilyl)amide (1.0 M in hexanes, 250 mL) dropwise over 40 minutes, keeping the reaction mixture temperature less than −73° C. The reaction was stirred at −77° C. for 2 hours, and bromine (12.5 mL) was added dropwise over 20 minutes, keeping the reaction mixture temperature less than −64° C. The reaction was stirred at −77° C. for 75 minutes and was quenched by the addition of cold 10% aqueous sodium thiosulfate (150 mL) to the −77° C. reaction. The mixture was warmed to room temperature and partitioned between half-saturated aqueous ammonium chloride and ethyl acetate. The layers were separated, and the organic layer was washed with water and brine, dried over sodium sulfate, filtered, and concentrated under reduced pressure. The residue was purified by silica gel chromatography, eluted with gradients of 80/20, 75/25, and 70/30 heptane/ethyl acetate to give the title compound. MS (DCI) m/z 299.0 and 301.0 (M+NH₃+H)⁺.

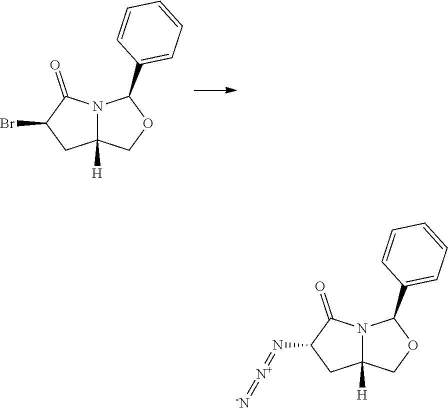

### Example 1.3.3

### (3R,6S,7aS)-6-azido-3-phenyltetrahydro-3H,5H-pyrrolo[1,2-c][1,3]oxazol-5-one

To a mixture of Example 1.3.2 (19.3 g) in N,N-dimethylformamide (100 mL) was added sodium azide (13.5 g). The reaction was heated to 60° C. for 2.5 hours. The reaction was cooled to room temperature and quenched by the addition of water (500 mL) and ethyl acetate (200 mL). The layers were separated, and the organic layer was washed brine. The combined aqueous layers were back-extracted with ethyl acetate (50 mL). The combined organic layers were dried with sodium sulfate, filtered, and concentrated under reduced pressure. The residue was purified by silica gel chromatography, eluted with 78/22 heptane/ethyl acetate, to give the title compound. MS (DCI) m/z 262.0 (M+NH₃+H)⁺.

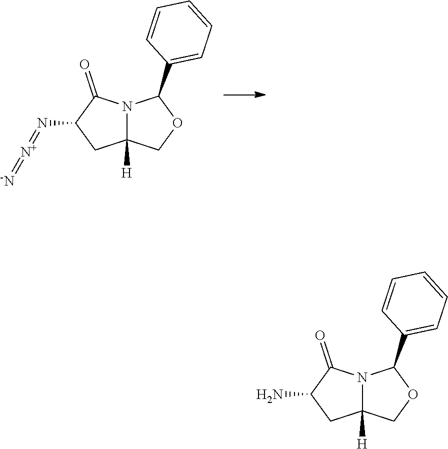

### Example 1.3.4

### (3R,6S,7aS)-6-amino-3-phenyltetrahydro-3H,5H-pyrrolo[1,2-c][1,3]oxazol-5-one

To a mixture of Example 1.3.3 (13.5 g) in tetrahydrofuran (500 mL) and water (50 mL) was added polymer-supported triphenylphosphine (55 g, Aldrich catalog #366455, loading—3 mmol/g). The reaction mixture was mechanically stirred overnight at room temperature. The reaction mixture was filtered through diatomaceous earth, eluted with ethyl acetate and toluene. The mixture was concentrated under reduced pressure, dissolved in dichloromethane (100 mL), dried with sodium sulfate, then filtered and concentrated to give the title compound, which was used in the subsequent step without further purification. MS (DCI) m/z 219.0 (M+H)⁺.

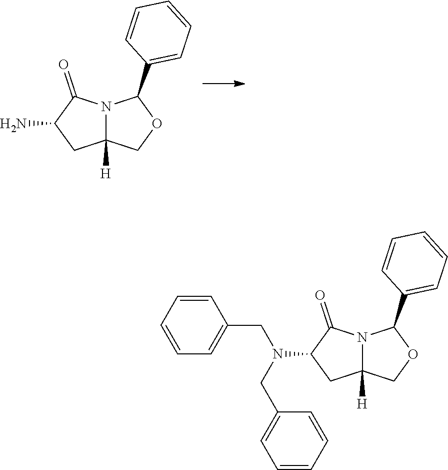 [SMILES: N[C@H]1C[C@H]2CO[C@H](c3ccccc3)N2C1=O ; O=C1[C@@H](N(Cc2ccccc2)Cc2ccccc2)C[C@H]2CO[C@H](c3ccccc3)N12]

### Example 1.3.5

### (3R,6S,7aS)-6-(dibenzylamino)-3-phenyltetrahydro-3H,5H-pyrrolo[1,2-c][1,3]oxazol-5-one

To a mixture of Example 1.3.4 (11.3 g) in N,N-dimethylformamide (100 mL) was added potassium carbonate (7.0 g), potassium iodide (4.2 g), and benzyl bromide (14.5 mL). The reaction mixture was stirred at room temperature overnight and quenched by the addition of water and ethyl acetate. The layers were separated, and the organic layer was washed with brine. The combined aqueous layers were back-extracted with ethyl acetate. The combined organic layers were dried with sodium sulfate, filtered, and concentrated under reduced pressure. The residue was purified by silica gel chromatography, eluted with a gradient of 10 to 15% ethyl acetate in heptane to give a solid that was triturated with heptane to give the title compound. MS (DCI) m/z 399.1 (M+H)⁺.

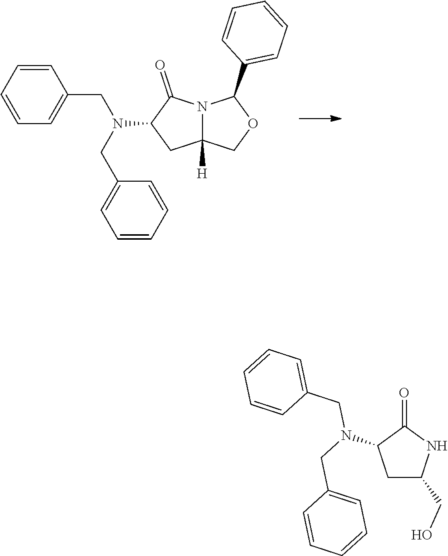 [SMILES: O=C1[C@@H](N(Cc2ccccc2)Cc2ccccc2)C[C@H]2CO[C@H](c3ccccc3)N12 ; O=C1N[C@H](CO)C[C@@H]1N(Cc1ccccc1)Cc1ccccc1]

### Example 1.3.6

### (3S,5S)-3-(dibenzylamino)-5-(hydroxymethyl)pyrrolidin-2-one

To a mixture of Example 1.3.5 (13 g) in tetrahydrofuran (130 mL) was added para-toluene sulfonic acid monohydrate (12.4 g) and water (50 mL), and the reaction was heated to 65° C. for 6 days. The reaction was cooled to room temperature and quenched by the addition of saturated aqueous sodium bicarbonate and ethyl acetate. The layers were separated, and the organic layer was washed with brine. The combined aqueous layers were back-extracted with ethyl acetate. The combined organic layers were dried with sodium sulfate, filtered, and concentrated under reduced pressure. The waxy solids were triturated with heptane (150 mL) to give the title compound. MS (DCI) m/z 311.1 (M+H)⁺.

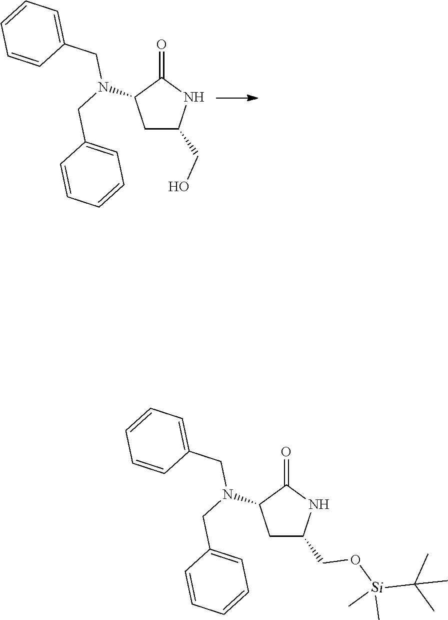 [SMILES: O=C1N[C@H](CO)C[C@@H]1N(Cc1ccccc1)Cc1ccccc1 ; CC(C)(C)[Si](C)(C)OC[C@@H]1C[C@H](N(Cc2ccccc2)Cc2ccccc2)C(=O)N1]

### Example 1.3.7

### (3S,5S)-5-({[tert-butyl(dimethyl)silyl]oxy}methyl)-3-(dibenzylamino)pyrrolidin-2-one

To a mixture of Example 1.3.6 (9.3 g) and 1H-imidazole (2.2 g) in N,N-dimethylformamide was added tert-butylchlorodimethylsilane (11.2 mL, 50 weight % in toluene), and the reaction mixture was stirred overnight. The reaction mixture was quenched by the addition of water and diethyl ether. The layers were separated, and the organic layer was washed with brine. The combined aqueous layers were back-extracted with diethyl ether. The combined organic layers were dried with sodium sulfate, filtered, and concentrated under reduced pressure. The residue was purified by silica gel chromatography, eluted with 35% ethyl acetate in heptane, to give the title compound. MS (DCI) m/z 425.1 (M+H)⁺.

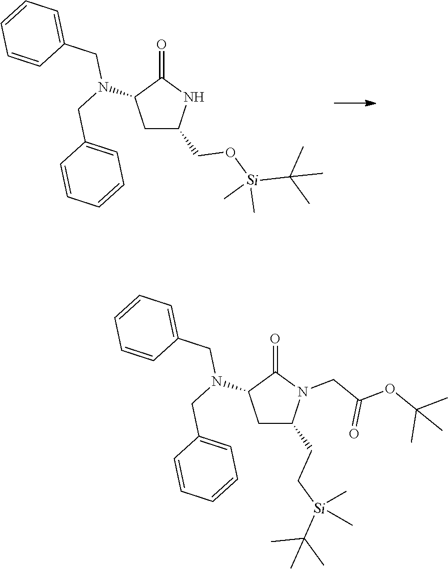 [SMILES: CC(C)(C)[Si](C)(C)OC[C@@H]1C[C@H](N(Cc2ccccc2)Cc2ccccc2)C(=O)N1 ; CC(C)(C)OC(=O)CN1C(=O)[C@@H](N(Cc2ccccc2)Cc2ccccc2)C[C@H]1CC[Si](C)(C)C(C)(C)C]

### Example 1.3.8

### tert-butyl [(3S,5S)-5-({[tert-butyl(dimethyl)silyl]oxy}methyl)-3-(dibenzylamino)-2-oxopyrrolidin-1-yl]acetate

To a cold (0° C.) mixture of Example 1.3.7 (4.5 g) in tetrahydrofuran (45 mL) was added 95% sodium hydride (320 mg) in two portions. The cold mixture was stirred for 40 minutes, and tert-butyl 2-bromoacetate (3.2 mL) was added. The reaction mixture was warmed to room temperature and stirred overnight. The reaction was quenched by the addition of water and ethyl acetate. The layers were separated, and the organic layer was washed with brine. The combined aqueous layers were back-extracted with ethyl acetate. The combined organic layers were dried with sodium sulfate, filtered, and concentrated under reduced pressure. The residue was purified by silica gel chromatography, eluted with a gradient of 5-12% ethyl acetate in heptane, to give the title compound. MS (DCI) m/z 539.2 (M+H)⁺.

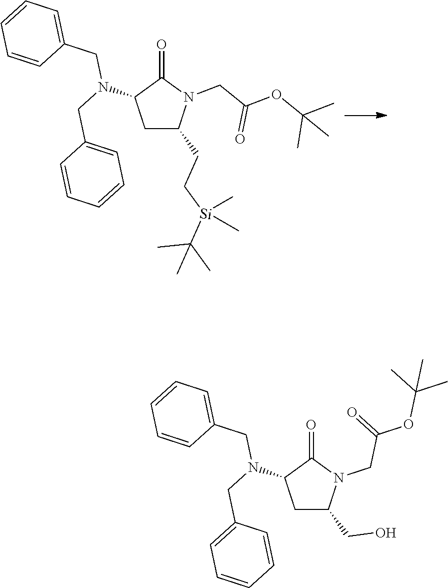 [SMILES: CC(C)(C)OC(=O)CN1C(=O)[C@@H](N(Cc2ccccc2)Cc2ccccc2)C[C@H]1CC[Si](C)(C)C(C)(C)C ; CC(C)(C)OC(=O)CN1C(=O)[C@@H](N(Cc2ccccc2)Cc2ccccc2)C[C@H]1CO]

### Example 1.3.9

### tert-butyl [(3S,5S)-3-(dibenzylamino)-5-(hydroxymethyl)-2-oxopyrrolidin-1-yl]acetate

To a mixture of Example 1.3.8 (5.3 g) in tetrahydrofuran (25 mL) was added tetrabutylammonium fluoride (11 mL, 1.0 M in 95/5 tetrahydrofuran/water). The reaction mixture was stirred at room temperature for one hour and then quenched by the addition of saturated aqueous ammonium chloride, water and ethyl acetate. The layers were separated, and the organic layer was washed with brine. The combined aqueous layers were back-extracted with ethyl acetate. The combined organic layers were dried with sodium sulfate, filtered, and concentrated under reduced pressure. The residue was purified by silica gel chromatography, eluted with 35% ethyl acetate in heptane, to give the title compound. MS (DCI) m/z 425.1 (M+H)⁺.

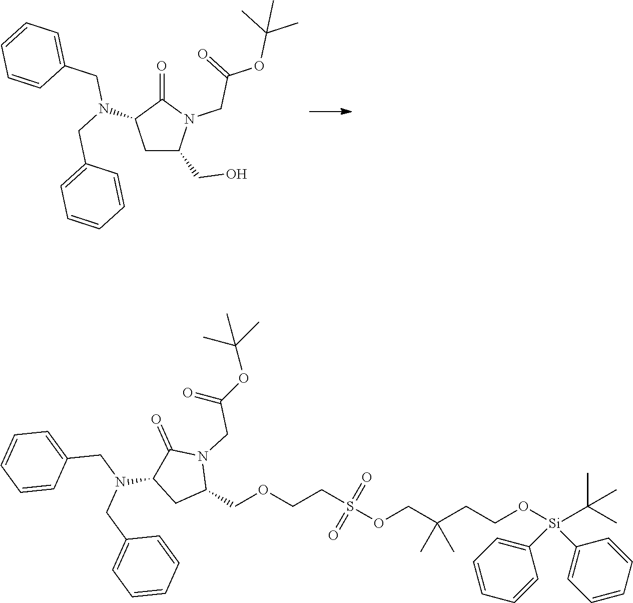 [SMILES: CC(C)(C)OC(=O)CN1C(=O)[C@@H](N(Cc2ccccc2)Cc2ccccc2)C[C@H]1CO ; CC(C)(CCO[Si](c1ccccc1)(c1ccccc1)C(C)(C)C)COS(=O)(=O)CCOC[C@@H]1C[C@H](N(Cc2ccccc2)Cc2ccccc2)C(=O)N1CC(=O)OC(C)(C)C]

### Example 1.3.10

### tert-butyl [(3S,5S)-3-(dibenzylamino)-2-oxo-5-(8,8,13,13-tetramethyl-5,5-dioxo-12,12-diphenyl-2,6,11-trioxa-5λ⁶-thia-12-silatetradecan-1-yl)pyrrolidin-1-yl]acetate

To a mixture of Example 1.3.9 (4.7 g) in dimethyl sulfoxide (14 mL) was added a mixture of 4-((tert-butyldiphenylsilyl)oxy)-2,2-dimethylbutyl ethenesulfonate (14.5 g) in dimethyl sulfoxide (14 mL). Potassium carbonate (2.6 g) and water (28 μL) were added, and the reaction mixture was heated at 60° C. under nitrogen for one day. The reaction was cooled to room temperature and then quenched by the addition of brine, water and diethyl ether. The layers were separated, and the organic layer was washed with brine. The combined aqueous layers were back-extracted with diethyl ether. The combined organic layers were dried with sodium sulfate, filtered, and concentrated under reduced pressure. The residue was purified by silica gel chromatography, eluted with a gradient of 15-25% ethyl acetate in heptane to give the title compound. MS (ESI+) m/z 871.2 (M+H)⁺.

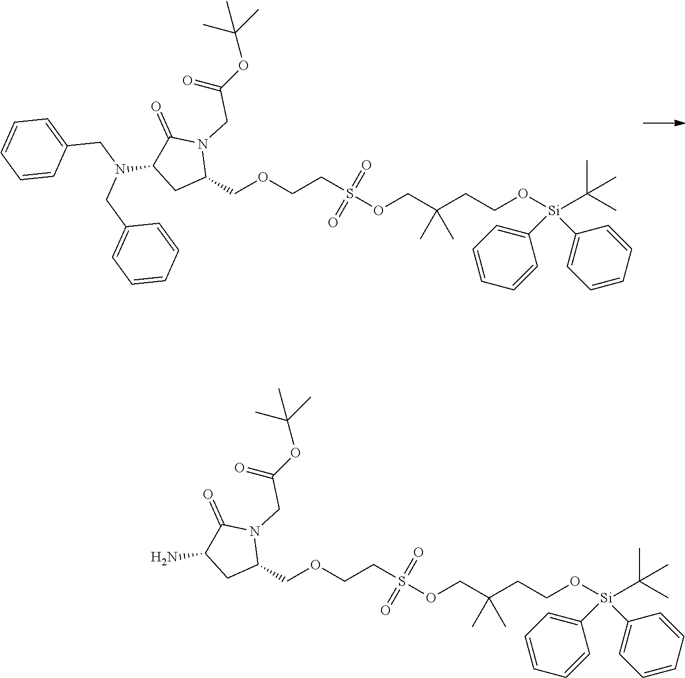 [SMILES: CC(C)(CCO[Si](c1ccccc1)(c1ccccc1)C(C)(C)C)COS(=O)(=O)CCOC[C@@H]1C[C@H](N(Cc2ccccc2)Cc2ccccc2)C(=O)N1CC(=O)OC(C)(C)C ; CC(C)(CCO[Si](c1ccccc1)(c1ccccc1)C(C)(C)C)COS(=O)(=O)CCOC[C@@H]1C[C@H](N)C(=O)N1CC(=O)OC(C)(C)C]

### Example 1.3.11

### tert-butyl [(3S,5S)-3-amino-2-oxo-5-(8,8,13,13-tetramethyl-5,5-dioxo-12,12-diphenyl-2,6,11-trioxa-5λ⁶-thia-12- silatetradecan-1-yl)pyrrolidin-1-yl]

Example 1.3.10 (873 mg) was dissolved in ethyl acetate (5 mL) and methanol (15 mL), and palladium hydroxide on carbon, 20% by weight (180 mg) was added. The reaction mixture was stirred under a hydrogen atmosphere (30 psi) at room temperature for 30 hours, then at 50° C. for one hour. The reaction mixture was cooled to room temperature, filtered, and concentrated to give the title compound. MS (ESI+) m/z 691.0 (M+H)⁺.

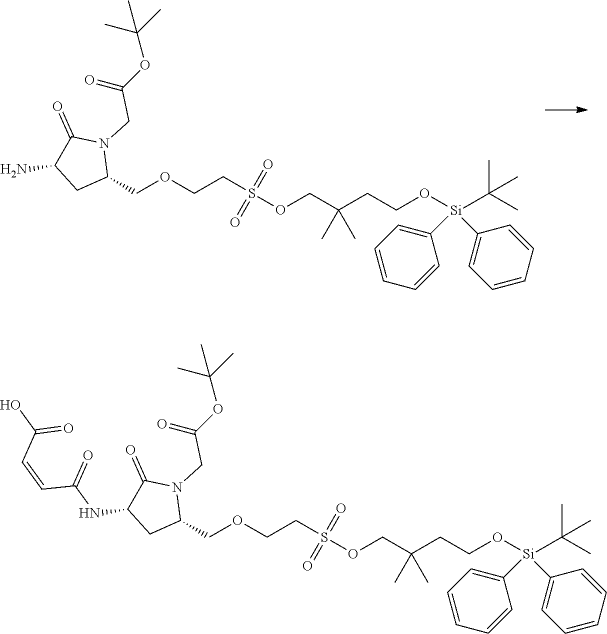 [SMILES: CC(C)(CCO[Si](c1ccccc1)(c1ccccc1)C(C)(C)C)COS(=O)(=O)CCOC[C@@H]1C[C@H](N)C(=O)N1CC(=O)OC(C)(C)C ; CC(C)(CCO[Si](c1ccccc1)(c1ccccc1)C(C)(C)C)COS(=O)(=O)CCOC[C@@H]1C[C@H](NC(=O)/C=C\C(=O)O)C(=O)N1CC(=O)OC(C)(C)C]

### Example 1.3.12

### (2Z)-4-{[(3S,5S)-1-(2-tert-butoxy-2-oxoethyl)-2-oxo-5-(8,8,13,13-tetramethyl-5,5-dioxo-12,12-diphenyl-2,6,11- trioxa-5λ⁶-thia-12-silatetradecan-1-yl)pyrrolidin-3-yl]amino}-4-oxobut-2-enoic Acid

Maleic anhydride (100 mg) was dissolved in dichloromethane (0.90 mL), and a mixture of Example 1.3.11 (650 mg) in dichloromethane (0.90 mL) was added dropwise followed by heating at 40° C. for 2 hours. The reaction mixture was directly purified by silica gel chromatography, eluted with a gradient of 1.0-2.5% methanol in dichloromethane containing 0.2% acetic acid. After concentrating the product-bearing fractions, toluene (10 mL) was added, and the mixture was concentrated again to give the title compound. MS (ESI-) m/z 787.3 (M−H)⁻.

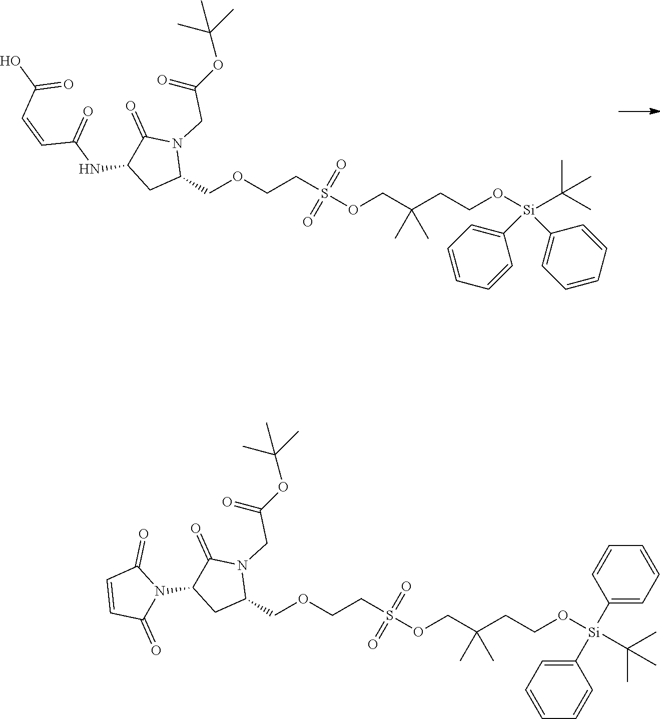 [SMILES: CC(C)(CCO[Si](c1ccccc1)(c1ccccc1)C(C)(C)C)COS(=O)(=O)CCOC[C@@H]1C[C@H](NC(=O)/C=C\C(=O)O)C(=O)N1CC(=O)OC(C)(C)C ; CC(C)(CCO[Si](c1ccccc1)(c1ccccc1)C(C)(C)C)COS(=O)(=O)CCOC[C@@H]1C[C@H](N2C(=O)C=CC2=O)C(=O)N1CC(=O)OC(C)(C)C]

### Example 1.3.13

### tert-butyl [(3S,5S)-3-(2,5-dioxo-2,5-dihydro-1H-pyrrol-1-yl)-2-oxo-5-(8,8,13,13-tetramethyl-5,5-dioxo-12,12-diphenyl- 2,6,11-trioxa-5λ⁶-thia-12-silatetradecan-1-yl)pyrrolidin-1-yl]acetate

Example 1.3.12 (560 mg) was slurried in toluene (7 mL), and triethylamine (220 μL) and sodium sulfate (525 mg) were added. The reaction was heated at reflux under a nitrogen atmosphere for 6 hours, and the reaction mixture was stirred at room temperature overnight. The mixture was filtered, and the solids were rinsed with ethyl acetate. The eluent was concentrated under reduced pressure, and the residue was purified by silica gel chromatography, eluted with 45/55 heptane/ethyl acetate to give the title compound.

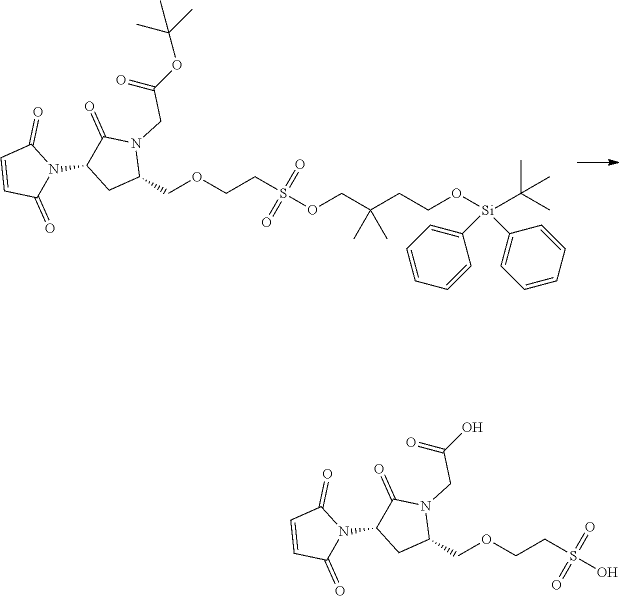 [SMILES: CC(C)(CCO[Si](c1ccccc1)(c1ccccc1)C(C)(C)C)COS(=O)(=O)CCOC[C@@H]1C[C@H](N2C(=O)C=CC2=O)C(=O)N1CC(=O)OC(C)(C)C ; O=C(O)CN1C(=O)[C@@H](N2C(=O)C=CC2=O)C[C@H]1COCCS(=O)(=O)O]

### Example 1.3.14

### {(3S,5S)-3-(2,5-dioxo-2,5-dihydro-1H-pyrrol-1-yl)-2-oxo-5-[(2-sulfoethoxy)methyl]pyrrolidin-1-yl}acetic Acid

Example 1.3.13 (1.2 g) was dissolved in trifluoroacetic acid (15 mL) and heated to 65-70° C. under nitrogen overnight. The trifluoroacetic acid was removed under reduced pressure. The residue was dissolved in acetonitrile (2.5 mL) and purified by preparative reverse-phase high-pressure liquid chromatography on a Phenomenex ® Luna® C18(2) AXIA™ column (250×50 mm, 10 μm particle size) using a gradient of 5-75% acetonitrile containing 0.1% trifluoroacetic acid in water (70 mL/minute) over 30 minutes, to give the title compound. MS (ESI-) m/z 375.2 (M−H)⁻.

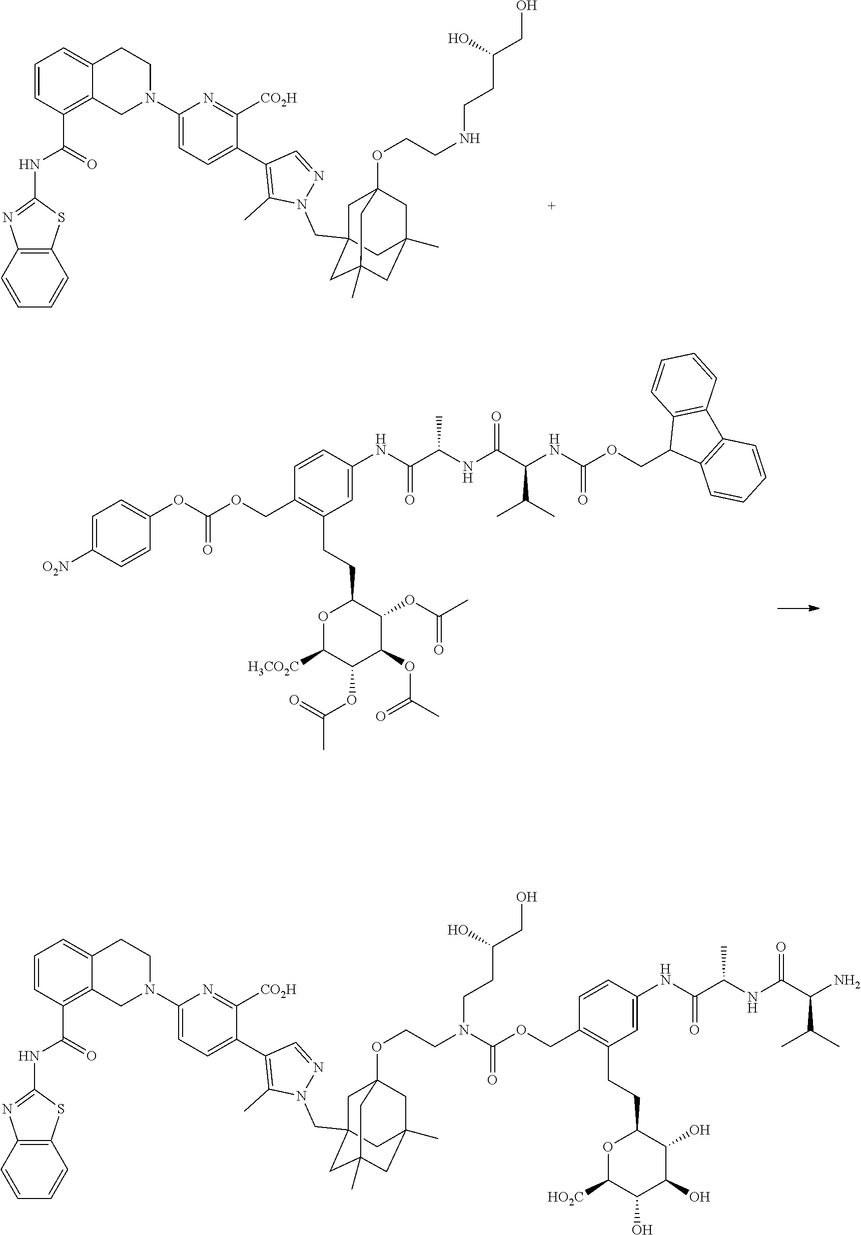 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCNCC[C@H](O)CO)(C3)C2 ; COC(=O)[C@H]1O[C@@H](CCc2cc(NC(=O)[C@H](C)NC(=O)[C@@H](NC(=O)OCC3c4ccccc4-c4ccccc43)C(C)C)ccc2COC(=O)Oc2ccc([N+](=O)[O-])cc2)[C@H](OC(C)=O)[C@@H](OC(C)=O)[C@@H]1OC(C)=O ; Cc1c(-c2ccc(N3CCc4cccc(C(=O)Nc5nc6ccccc6s5)c4C3)nc2C(=O)O)cnn1CC12CC3(C)CC(C)(C1)CC(OCCN(CC[C@H](O)CO)C(=O)OCc1ccc(NC(=O)[C@H](C)NC(=O)[C@@H](N)C(C)C)cc1CC[C@@H]1O[C@H](C(=O)O)[C@@H](O)[C@H](O)[C@H]1O)(C3)C2]

### Example 1.4.1

### 3-[1-({3-[2-({[(4-{[(2S)-2-{[(2S)-2-amino-3-methylbutanoyl]amino}propanoyl]amino}-2-{2-[(2S,3R,4R,5S,6S)-6-carboxy-3,4,5-trihydroxyoxan-2-yl]ethyl}phenyl)methoxy]carbonyl}[(3S)-3,4-dihydroxybutyl]amino)ethoxy]-5,7-dimethyladamantan-1-yl}methyl)-5-methyl-1H-pyrazol-4-yl]-6-{8-[(1,3-benzothiazol-2-yl)carbamoyl]-3,4- dihydroisoquinolin-2(1H)-yl}pyridine-2-carboxylic Acid

To a suitably sized reactor was charged Example 1.1.17 (5.17 g), Example 1.2.19 (6.99 g), N,N-dimethylformamide (50 mL) and N,N-diisopropylethylamine (7.6 mL). After the solids were dissolved, 1-hydroxybenzotriazole hydrate (1.21 g) was charged to the reactor, and the reaction progress was monitored by HPLC (Ascentis® Express® C18, 4.6×150 mm, 2.7 μm, 1.5 mL/minute flow rate, eluted with a gradient of 40 to 100% acetonitrile in 0.05% HClO₄ in water over 18 minutes). After coupling was determined to be complete, tetrahydrofuran (62 mL) was charged to the reactor, and the reaction mixture was cooled to 0° C. Lithium methoxide (62 mL, 1.0 M solution in methanol) was charged over 1 hour, and the reaction mixture was allowed to warm to ambient temperature. The reaction progress was monitored by HPLC (Ascentis® Express® C18, 4.6×150 mm, 2.7 μm, 1.5 mL/minute flow rate, eluted with a gradient of 40 to 100% acetonitrile in 0.05% HClO₄ in water over 18 minutes), and after hydrolysis was determined to be complete, acetonitrile (110 mL) was charged to the reactor over 2 hours. The slurry was allowed to stir for an additional 2 hours, and the solids were isolated via vacuum filtration, followed by washing the wet cake with acetonitrile (2×10 mL). The residue was purified via reverse phase chromatography (Phenomenex® Luna® C18, 50×250 mm, 10 μm, 80 mL/minute flow rate, 25 mM ammonium acetate/acetonitrile, 64/36 isocratic), and the desired fractions were lyophilized to give the title compound as an acetate salt. MS (ESI) m/z 1357.5 (M+H)⁺.

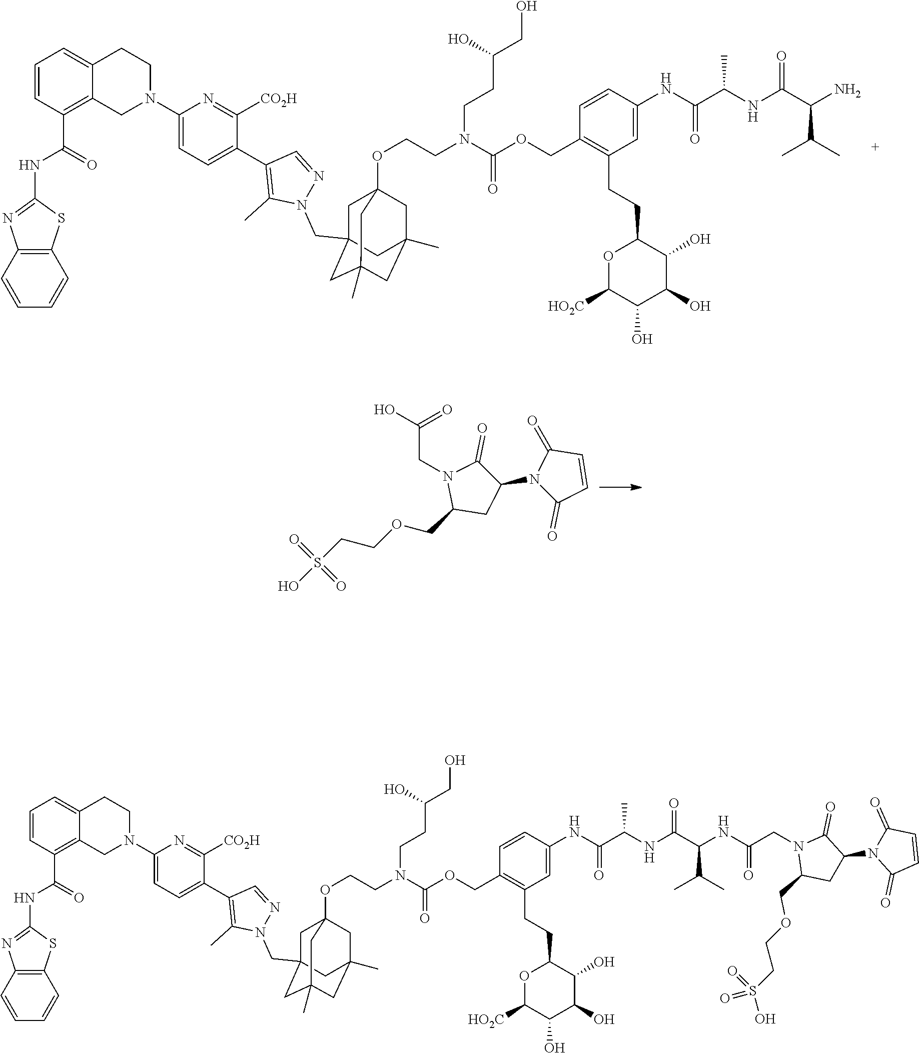

### Example 1

### 6-{[8-[(1,3-benzothiazol-2-yl)carbamoyl]-3,4-dihydroisoquinolin-2(1H)-yl}-3-[1-({3-[2-({[(2-{2-[(2S,3R,4R,5S,6S)-6-carboxy-3,4,5-trihydroxyoxan-2-yl]ethyl}-4-{[(2S)-2-{[(2S)-2-(2-{(3S,5S)-3-(2,5-dioxo-2,5-dihydro-1H-pyrrol-1-yl)-2-oxo-5-[(2-sulfoethoxy)methyl]pyrrolidin-1-yl}acetamido)-3- methylbutanoyl]amino}propanoyl]amino}phenyl)methoxy]carbonyl}[(3S)-3,4-dihydroxybutyl]amino)ethoxy]-5,7-dimethyladamantan-1-yl}methyl)-5- methyl-1H-pyrazol-4-yl]pyridine-2-carboxylic Acid (Synthon AAA)

Example 1.3.14 (17.7 mg) was dissolved in N,N-dimethylformamide (0.14 mL), and O-(7-azabenzotriazol-1-yl)-N,N,N′,N′-tetramethyluronium hexafluorophosphate (16.9 mg) and N,N-diisopropylethylamine (18.5 μL) were added. The mixture was stirred for 3 minutes at room temperature and then added to a mixture of Example 1.4.1 (52.0 mg) and N,N-diisopropylethylamine (24.7 μL) in N,N-dimethylformamide (0.2 mL). After 1 hour, the reaction was diluted with N,N-dimethylformamide/water 1/1 (1.0 mL) and purified by reverse-phase HPLC (Phenomenex® Luna® C18 250×50 mm column), eluted with 5-75% acetonitrile in 0.1% trifluoroacetic acid/water (100 mL/minute), to provide the title compound. ¹H NMR (500 MHz, dimethyl sulfoxide-d₆) δ ppm 9.86 (br d, 1H), 8.17 (br d, 1H), 8.04 (m, 2H), 7.78 (d, 1H), 7.61 (d, 1H), 7.51 (br d, 1H), 7.49-7.39 (m, 4H), 7.36 (m, 2H), 7.29 (s, 1H), 7.21 (d, 1H), 7.07 (s, 2H), 6.95 (d, 1H), 5.00 (s, 2H), 4.96 (s, 2H), 4.64 (t, 1H), 4.36 (m, 1H), 4.19 (m, 1H), 4.16 (d, 1H), 4.01 (d, 1H), 3.88 (br t, 2H), 3.82 (br m, 3H), 3.75 (br m, 1H), 3.64 (t, 2H), 3.54 (d, 2H), 3.47 (m, 4H), 3.43 (br m, 4H), 3.23 (br m, 5H), 3.13 (t, 1H), 3.10 (br m, 1H), 3.01 (br m, 2H), 2.93 (t, 1H), 2.83-2.68 (m, 3H), 2.37 (m, 1H), 2.08 (s, 3H), 1.99 (br m, 2H), 1.85 (m, 1H), 1.55 (br m, 1H), 1.37 (br m, 1H), 1.28 (br m, 6H), 1.10 (br m, 7H), 0.93 (br m, 1H), 0.88-0.69 (m, 12H); MS (ESI) m/z 1713.6 (M−H)⁻.

### Example 2

### Preparation of AM2 Antibody

The amino acid sequence of the VH region for AM2 is provided in SEQ ID NO: 22. The amino acid sequence of the VL region for AM2 is provided in SEQ ID NO: 23. The heavy chain of AM2 is provided as SEQ ID NO: 1 and the light chain is provided as SEQ ID NO: 5.

The full-length nucleic acid sequences heavy and light chains of AM2 were expressed by transiently transfecting expression vectors encoded the heavy and light chains of AM2 in HEK293 cells. The amino acid sequence of the leader sequence used for expression of the heavy chain was MEFGLSWLFLVAILKGVQC (SEQ ID NO: 25) while the amino acid sequence used for expression of the light chain was MDMRVPAQLLGLLLLWFPGSRC (SEQ ID NO: 26). AM2, having a heavy chain of SEQ ID NO: 1 and a light chain of SEQ ID NO: 5, was subsequently purified for subsequent functional assessment.

Relative to anti-EGFR antibody AM2B, which comprises a heavy chain of SEQ ID NO: 21 and a light chain of SEQ ID NO: 24, amino acid mutations in AM2 represent (1) human IgG allotype changes from a z, non-a allotype to a z,a allotype; (2) a C6v1 (LC:C214A) mutation, enabling site-specific conjugation, and (3) a LALA mutation (two leucine to alanine substitutions, L234A, L235A).

### Example 3

### Conjugation of Synthon AAA with EGFR AM2 Antibody

A 10 mM solution of 2-(diphenylphosphino) benzoic acid (DPPBA, Sigma Aldrich) was prepared in dimethylacetamide (DMA). 2.42 equivalents of DPPBA was added to a solution of EGFR AM2 antibody pre-equilibrated at 4° C. (˜10 mg/mL, formulated in 1× Dulbecco's phosphate-buffered saline (DPBS), pH 7.4 with 2 mM ethylenediaminetetraacetic acid (EDTA)). The reaction mixture was gently mixed and incubated at 4° C. for 16-24 hours. 3.7 Equivalents of 10 mM Synthon AAA (6-{8-[(1,3-benzothiazol-2-yl)carbamoyl]-3,4-dihydroisoquinolin-2(1H)-yl}-3-[1-({3-[2-({[(2-{2-[(2S,3R,4R,5S,6S)-6-carboxy-3,4,5-trihydroxyoxan-2-yl]ethyl}-4-{[(2S)-2-{[(2S)-2-(2-{(3S,5S)-3-(2,5-dioxo-2,5-dihydro-1H- pyrrol-1-yl)-2-oxo-5-[(2-sulfoethoxy)methyl]pyrrolidin-1-yl}acetamido)-3-methylbutanoyl]amino}propanoyl]amino}phenyl)methoxy]carbonyl}[(3S)-3,4-dihydroxybutyl]amino)ethoxy]-5,7-dimethyladamantan-1-yl}methyl)-5-methyl-1H-pyrazol-4yl]pyridine-2-carboxylic acid (see U.S. Patent Application Publication No. 2019/0343961) (dissolved in DMA) was added into the reduced antibody solution and gently mixed. The reaction mixture was incubated at room temperature for 60 minutes, and subsequently quenched by 2 equivalents of N-acetyl-L-cysteine (NAC, Sigma Aldrich A-8199-10G). The antibody-drug conjugate (ADC) was purified by Hydrophobic Interaction Chromatography (HIC).

### Example 4

### EGFR-Targeted ADCs Inhibit the Growth of Cancer Cells In Vivo

The NSCLC cell lines, EBC-1 and NCI-H441 (called H441 hereafter) were obtained from JCRB and ATCC, respectively. Cells were maintained in monolayer culture for at most 3 passages according to recommendations of the supplier. A suspension of 5×10⁶ cells in culture medium mixed with Matrigel (1:1, volume:volume) was injected subcutaneously in the right flank of female SCID/beige mice. Treatment started when the sizes of the flank tumors were approximately 200 mm³.

| | Antibody | Antibody | | | |
|---|---|---|---|---|---|
| | Heavy Chain | Light Chain | | | |
| | (SEQ ID | (SEQ ID | | Average | DAR |
| | NO) | NO) | Synthon | DAR | Method |
| AM2B-AAA | 21 | 24 | AAA | 2 | HIC |
| AM7-AAA | 9 | 11 | AAA | 2 | HIC |

Average DAR represents the average number of drugs coupled to the antibodies for the composition.

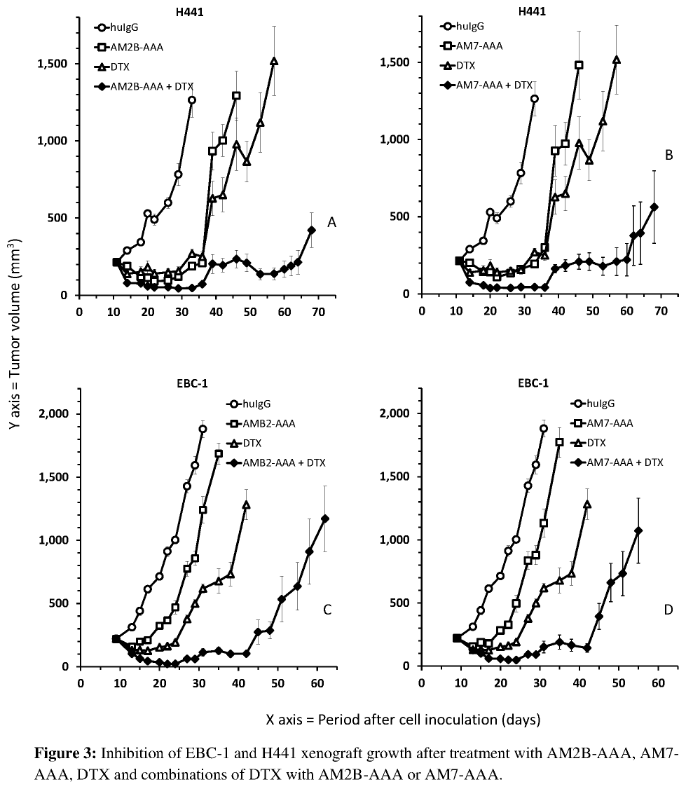

FIG. 3 shows AM2B-AAA and AM7-AAA inhibited growth of human NSCLC grown as xenografts in immune-compromised mice. Moderate growth inhibition was observed after administration of the ADCs as single agent. Durability of the inhibition caused by docetaxel (DTX) increased after coadministration with either AM2B-AAA or AM7-AAA. The activity of ADCs was compared to a non-targeting IgG antibody (AB095) (a human IgG1 antibody recognizing tetanus toxoid; see Larrick et al, 1992, Immunological Reviews 69-85).

FIGS. 3A and 3B show changes of tumor volume following treatment of the papillary adenocarcinoma, H441.

FIGS. 3C and 3D show changes of tumor volume following treatment of the squamous carcinoma, EBC-1.

Each point of the curve represents the mean volume of 8 tumors.

Error bars depict the standard error of the mean.

### Example 5

### FACS Analysis AM2-AAA Inhibits Bcl-xL in Cells Overexpressing EGFR

To assay binding of AM2-AAA to cell surface overexpressed wt EGFR and to mutant forms of EGFR including the activating mutations found in NSCLC (mutEGFR), tumor cells overexpressing wt EGFR (A431) and mutEGFR (NCI-H1650) were assessed by fluorescence activated cell sorting (FACS).

Cells were harvested from flasks when at approximately 1.5×10⁶ cells/mL. Cells were washed once in PBS/1% FBS (FACS buffer) then resuspended at 2.5×10⁶ cells/mL in FACS buffer. 50 μL of cells were added to a round bottom 96-well plate. 50 μL of a 2× concentration of mAb/ADC (final concentrations are indicated in the figures) was added to wells and the plate was incubated at 4° C. for one hour. The cells were washed twice in FACS buffer and resuspended in 50 μL of a 1:100 dilution of secondary Ab (AlexaFluor 488, Invitrogen, 11013) diluted in FACS buffer. The plate was incubated at 4° C. for one hour and washed twice with FACS buffer. Cells were resuspended in 100 μL of PBS/1% formaldehyde and analyzed on a Becton Dickinson FACSCanto II flow cytometer. Data was analyzed using WinList flow cytometry analysis software.

| | Antibody | Antibody | | | |
|---|---|---|---|---|---|
| | Heavy Chain | Light Chain | | | |
| | (SEQ ID | (SEQ ID | | Average | DAR |
| | NO) | NO) | Synthon | DAR | Method |
| AM2 | 1 | 5 | — | — | — |
| AM2-AAA | 1 | 5 | AAA | 2 | HIC |
| MSL109 hIgG | 29 | 30 | — | — | — |

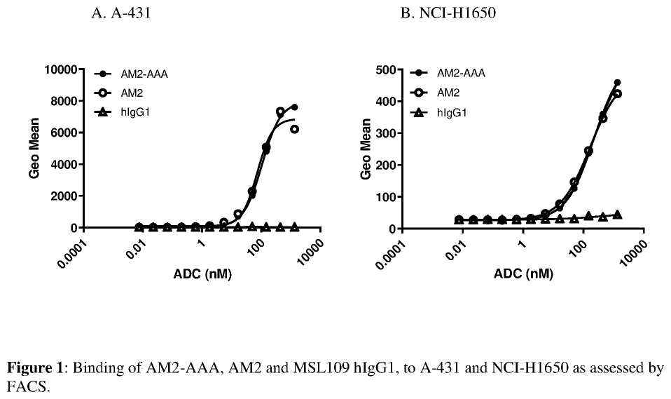

AM2-AAA and AM2 were shown to bind to both cell lines with similar apparent affinity, indicating no impact of the linker drug on Ab binding properties. No binding was observed using a non-binding control MSL109 hIgG (FIG. 1). Monoclonal antibody to CMV glycoprotein H (MSL109) is a non-targeting control that has a heavy chain set forth as SEQ ID NO: 29 and a light chain as set forth as SEQ ID NO: 30.

### Example 6

### BIM-Bcl-xL Complex Disruption Assay

To assess if treatment of cells with AM2-AAA inhibited Bcl-xL in an EGFR-dependent manner, disruption of Bcl-xL-BIM complexes in both wt EGFR and mutEGFR expressing cell lines was assessed.

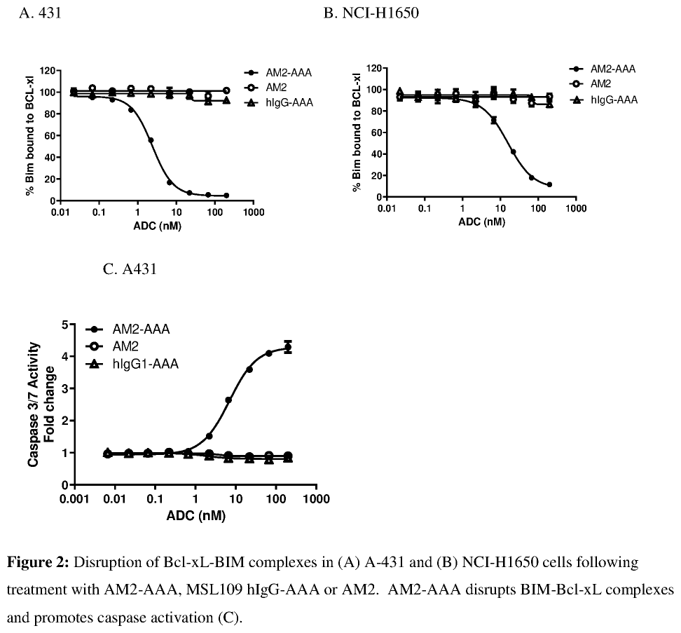

Cells were plated at 50×10³ cells/well in 96-well plates in growth media RPMI-1640 (GibcoInvitrogen, 22400-089) supplemented with 10% fetal bovine serum in the morning. In the afternoon, treatments were added in fresh media to triplicate wells. Twenty-four hours later, cells were lysed with 10 mM HEPES, 150 mM NaCl, 1% CHAPS buffer and Bcl-xL/BIM complexes in protein lysates were captured on plates (MesoScale Diagnostics LLC, L15SA-1) previously coated with anti-Bcl-xL capture antibody (R&D systems, biotinylated anti-Bcl-xL 840768, DYC894-2 kit). Plates were washed with PBS and detection antibody anti-BIM (Cell Signaling, 2933) was added to wells for one hour at room temperature. Plates were then washed with PBS and Sulfo-tagged anti rabbit antibody (MesoScale Diagnostics LLC, R32AB-1) was added to wells, and then incubated at room temperature for one hour. Plates were washed and MSD read buffer (MesoScale Diagnostics LLC, R92TC-2) was added to wells and plates were read on MesoScale Diagnostics LLC instrument (Sector S 600). Data was plotted as percent remaining BIM/BCL-xL complex. (FIG. 2)

### Example 7

### Caspase Assay

| | Antibody | Antibody | | | |
|---|---|---|---|---|---|
| | Heavy Chain | Light Chain | | | |
| | (SEQ ID | (SEQ ID | | Average | DAR |
| | NO) | NO) | Synthon | DAR | Method |
| AM2 | 1 | 5 | — | — | — |
| AM2-AAA | 1 | 5 | AAA | 2 | HIC |
| MSL109 | 29 | 30 | AAA | 1.6 | MS |
| hIgG-AAA | | | | | |

A431 cells were plated at 50,000 cells per well in 96 well plates (Costar, 3610) in growth media. After 24 hours in culture at 37° C., ADCs were added to wells and incubated at 37° C. in a CO₂ incubator for 24 hours. After incubation, 100 μL of Caspase-Glo 3/7 Assay reagent (Promega, G8093) was added to each well and shaken for 10 minutes. Plates were then incubated at 37° C. for 20 minutes. Caspase 3/7 activity was assessed using a Victor luminescence plate reader (Perkin Elmer).

While a non-targeting ADC (MSL109 hIgG-AAA) or AM2 failed to disrupt Bcl-xL-BIM complexes, AM2-AAA treatment resulted in efficient complex disruption in both cell lines. These results indicate that the AAA warhead was specifically delivered via AM2-AAA to EGFR expressing cells (See FIG. 2) and inhibited Bcl-xL activity.

The ability of AM2-AAA to promote caspase activation, a downstream consequence of Bcl-xL inhibition, was also assessed in the A-431 cells. AM2-AAA, but not AM2 or the non-targeting MSL109 hIgG-AAA induced caspase activation supporting EGFR-dependent on mechanism activity of the targeted ADC.

### Example 8

### Toxicity Study

| | Antibody | Antibody | | | |
|---|---|---|---|---|---|
| | Heavy Chain | Light Chain | | | |
| | (SEQ ID | (SEQ ID | | Average | DAR |
| | NO) | NO) | Synthon | DAR | method |
| AM2B-AAA | 21 | 24 | AAA | 1.97 | HIC |
| AM2-AAA | 1 | 5 | AAA | 2 | MS |

Study A: Four Week Toxicity Study of two Intravenous Doses (Once Every Three Weeks) of AM2B-AAA in Cynomolgus Monkeys

Two intravenous administrations of AM2B-AAA were administered to male and female cynomolgus monkeys in four groups: control (0 mg/kg/dose), dose A (low; X mg/kg/dose), dose B (mid; 3X mg/kg/dose), and dose C (high; 6X mg/kg/dose). Administration of AM2B-AAA resulted in adverse findings in the arteries of multiple organs at the high dose (dose C). Inflammation, artery (minimal to moderate) at Dose C, with positive immunohistochemical staining for human IgG and complement, consistent with immune complex disease secondary to administration of foreign protein, was observed. This was interpreted as secondary to immune complex formation and deposition with fixation of complement, as demonstrated by immunohistochemistry. Non-adverse findings attributed to AB2B-AAA included increased glomerular matrix in the kidney at all dose levels.

Noteworthy test item-related changes considered not adverse due to low magnitude and absence of functional effect included kidney [increase, glomerular matrix (minimal to mild) at ≥Dose A); dilation, tubules (minimal) at >Dose B]. RBC mass (decrease; minimal at ≥Dose B); platelet count (decrease; mild to moderate at ≥Dose B); acute phase inflammatory response characterized by globulin (minimal increase at Dose A and Dose B; mild to moderate increase at Dose C), CRP (mild increase at Dose C), albumin (minimal to mild decrease at >Dose B), and fibrinogen (minimal increase at >Dose B).

Study B: A Five-Week (two Dose; Q3W) Intravenous Exploratory Toxicity Study of AM2-AAA in Cynomolgus Monkeys

Two intravenous administrations of AM2-AAA were administered to male and female cynomolgus monkeys at 0 mg/kg/dose (control), Dose 1 (6X mg/kg/dose, where X is the same as in Study A), and Dose 2 (15X mg/kg/dose). There was no finding of immune complex disease detected.

Kidney findings consisted of mild to moderate increases in glomerular matrix with accompanying mild increases in urea nitrogen at ≥Dose 1. Hematology findings included test-item related mild to moderate decreases in RBC mass, non-adverse reticulocyte decreases, and moderate to marked platelet decreases at ≥Dose 1 (considered adverse only at Dose 2). Other test item-related changes considered not adverse included mildly to moderately decreased number of lymphocytes in the thymus at ≥Dose 1, minimally to mildly increased AST activity at ≥Dose 1, increased bilirubin at Dose 1, mildly to moderately decreased calcium at ≥Dose 1, and minimally to mildly decreased albumin at ≥Dose 1.

### Example 9

### Biacore Binding Analysis of AM2 and AM2-AAA

Biacore analysis was performed to compare the affinity of the AM2 antibody and the AM2-AAA ADC to three forms of recombinant EGFR, specifically the wild-type EGFR extra-cellular domain (ECD) (EGFR(h)(1-645)), EGFRvIII (EGFR(h)(1-29)-G-(298-645)) and a truncated wild-type EGFR 1-525 (EGFR1(h)(1-525)). AM2 includes the heavy and light chain amino acid sequences of AM2 as provided in SEQ ID NOs: 1 and 5, respectively, and was made according to standard methods. AM2-AAA comprises the AM2 antibody having the heavy and light chain amino acid sequences provided in SEQ ID NOs: 1 and 5, respectively, conjugated to the AAA synthon (average DAR 2).

Binding kinetics for AM2 and AM2-AAA for recombinant soluble EGFR extracellular domains (ECDs) were determined by surface plasmon resonance-based measurements made on a Biacore T200 instrument (GE Healthcare) at 25° C. using an anti-Fc capture assay approach. Recombinant soluble ECDs for the three isoforms of EGFR were expressed from transiently transfected HEK293 cells as secreted proteins with a C-terminal myc and histidine tag, and purified by Ni-IMAC (immobilized metal affinity chromatography) and SEC. In particular, the EGFR ECD tested included amino acids 1-645 of EGFR fused to a myc and histidine tag [(EGFR (1-645)-LESRGPF-Myc-NMHTG-6His (“LESRGPF” (SEQ ID NO: 27))]. The EGFRvIII variant was also fused to a myc and histidine tag (EGFR(h)(1-29)-G-(298-645)-LESRGPF-Myc-NMHTG-6His), as was the ECD EGFR 1-525 [EGFR 1(h)(1-525)]-LESRGPF-Myc-NMHTG-6His (“LESRGPF” (SEQ ID NO: 28))]. All ECDs were expressed with the signal sequence MRPSGTAGAALLALLAALCPASRA which was cleaved during secretion.

Chip preparation and binding kinetic measurements were made in the assay buffer HBS-EP+ (10 mM Hepes, pH 7.4, 150 mM NaCl, 3 mM EDTA, 0.05% Tween 20). For anti-Fc capture chip preparation, approximately 2000 RU of goat anti-human IgG Fc polyclonal antibody (Thermo Fisher Scientific Inc., cat. 31125), diluted to 25 μg/mL in 10 mM sodium acetate (pH 4.5) was directly immobilized across a CMS biosensor chip using a standard amine coupling kit according to manufacturer's instructions and procedures (GE Healthcare). Unreacted moieties on the biosensor surface were blocked with ethanolamine. For binding kinetics measurements each assay cycle consisted of the following steps: (1) capture of test antibody on test surface only; (2) analyte injection (EGFR ECD or buffer only) over both reference and test surface (240 μL at 80 μl/min), after which the dissociation was monitored for 900 seconds at 80 μl/min; (3) regeneration of the capture surface by 10 mM Glycine-HCl, pH 1.5 injections over both reference and test surface.

For kinetic determinations, analytes were randomized 3-fold dilution series from 3 uM top dose. During the assay, all measurements were referenced against the capture surface alone (i.e., with no captured test antibody) and buffer-only injections were used for secondary referencing. Data were processed and fitted globally to a 1:1 binding model using Biacore T200 Evaluation software to determine the binding kinetic rate constants, kₐ (M⁻¹s⁻¹) and k_{d} (s⁻¹), and the equilibrium dissociation constant K_{D} (M). Results of the Biacore analysis are shown in Table 1.

TABLE 1
Binding Kinetics of AM2 Antibody and AM2-AAA ADC

| EGFRvIII (hEGFR de2-7) | | | EGFRs1-501 (EGFR(1-525)) | | | hEGFR WT | | |
|---|---|---|---|---|---|---|---|---|
| [EGFR(H)(1-29)-G-(298-645)] | | | [EGFR1(h)(1-525)] | | | [EGFR(1-645)] | | |
| kₐ | k_{d} | K_{D} | kₐ | k_{d} | K_{D} | kₐ | k_{d} | K_{D} |
| (1/Ms) | (1/s) | (M) | (1/Ms) | (1/s) | (M) | (1/Ms) | (1/s) | (M) |

| | | | | | | | |
|---|---|---|---|---|---|---|---|
| AM2 | 3.3 × 10⁴ | 1.7 × 10⁻⁴ | 5.0 × 10⁻⁹ | 2.8 × 10³ | 1.2 × 10⁻³ | 4.2 × 10⁻⁷ | No observable binding |
| | | | | | | | (at 3 μM top dose) |
| AM2- | 3.5 × 10⁴ | 1.7 × 10⁻⁴ | 4.9 × 10⁻⁹ | 2.9 × 10³ | 1.2 × 10⁻³ | 4.0 × 10⁻⁷ | No observable binding |
| AAA | | | | | | | (at 3 μM top dose) |

While various specific embodiments have been illustrated and described, it will be appreciated that various changes can be made without departing from the spirit and scope of the disclosure.

TABLE 2
Sequence Table

| SEQ | | | |
|---|---|---|---|
| ID | | Protein | |
| NO | Clone | Region | V Region |
| 1 | AM2 | | EVQLQESGPGLVKPSQTLSLTCTVS [symbol] WIRQPPG |
| | Heavy | | KGLEWMG [symbol] RITISRDTSKNQFFLKLNSV |
| | Chain | | TAADTATYYCVT [symbol] WGQGTLVTVSSASTKGPSVFPL |
| | | | APSSKSTSGGTAALGCLVKDYFPEPVTVSWNSGALTSGVHTF |
| | | | PAVLQSSGLYSLSSVVTVPSSSLGTQTYICNVNHKPSNTKVDK |
| | | | KVEPKSCDKTHTCPPCPAPEAAGGPSVFLFPPKPKDTLMISRT |
| | | | PEVTCVVVDVSHEDPEVKFNWYVDGVEVHNAKTKPREEQY |
| | | | NSTYRVVSVLTVLHQDWLNGKEYKCKVSNKALPAPIEKTISK |
| | | | AKGQPREPQVYTLPPSRDELTKNQVSLTCLVKGFYPSDIAVE |
| | | | WESNGQPENNYKTTPPVLDSDGSFFLYSKLTVDKSRWQQGN |
| | | | VFSCSVMHEALHNHYTQKSLSLSPGK |
| 2 | AM2 | HC CDR1 | [symbol] |
| 3 | AM2 | HC CDR2 | [symbol] |
| 4 | AM2 | HC CDR3 | [symbol] |
| 5 | AM2 | | DIQMTQSPSSMSVSVGDRVTITC [symbol] |
| | Light | | [symbol] WLQQKPGKSFKGLIY [symbol] GVPSRFSG |
| | Chain | | SGSGTDYTLTISSLQPEDFATYYC [symbol] |
| | | | [symbol] FGGGTKLEIKRTVAAPSVFIFPPSDEQLKSGT |
| | | | ASVVCLLNNFYPREAKVQWKVDNALQSGNS |
| | | | QESVTEQDSKDSTYSLSSTLTLSKADYEKHKV |
| | | | YACEVTHQGLSSPVTKSFNRGEA |
| 6 | AM2 | LC CDR1 | [symbol] |
| 7 | AM2 | LC CDR2 | [symbol] |
| 8 | AM2 | LC CDR3 | [symbol] |
| 9 | AM7 | | EVQLQESGPGLVKPSQTLSLTCTVS [symbol] WIRQLP |
| | Heavy | | GKGLEWMG [symbol] RITISRDTSKNQFFLKLNS |
| | Chain | | VTAADTATYYCVT [symbol] WGQGTLVTVSSASTKGPSVFPL |
| | | | APSSKSTSGGTAALGCLVKDYFPEPVTVSWNSGALTSGVHTF |
| | | | PAVLQSSGLYSLSSVVTVPSSSLGTQTYICNVNHKPSNTKVDK |
| | | | KVEPKSCDKTHTCPPCPAPEAAGGPSVFLFPPKPKDTLMISRT |
| | | | PEVTCVVVDVSHEDPEVKFNWYVDGVEVHNAKTKPREEQY |
| | | | NSTYRVVSVLTVLHQDWLNGKEYKCKVSNKALPAPIEKTISK |
| | | | AKGQPREPQVYTLPPSRDELTKNQVSLTCLVKGFYPSDIAVE |
| | | | WESNGQPENNYKTTPPVLDSDGSFFLYSKLTVDKSRWQQGN |
| | | | VFSCSVMHEALHNHYTQKSLSLSPGK |
| 2 | AM7 | HC CDR1 | [symbol] |
| 3 | AM7 | HC CDR2 | [symbol] |
| 10 | AM7 | HC CDR3 | [symbol] |
| 11 | AM7 | | DIQMTQSPSSMSVSVGDRVTITC [symbol] WLQQKPGK |
| | Light | | SFKGLIY [symbol] GVPSRFSGSGSGTDYTLTISSLQPEDFAT |
| | Chain | | YYC [symbol] FGGGTKLEIKRTVAAPSVFIFPPSDEQLKSG |
| | | | TASVVCLLNNFYPREAKVQWKVDNALQSGNSQESVTEQDSK |
| | | | DSTYSLSSTLTLSKADYEKHKVYACEVTHQGLSSPVTKSFNR |
| | | | GEA |
| 12 | AM7 | LC CDR1 | [symbol] |
| 13 | AM7 | LC CDR2 | [symbol] |
| 14 | AM7 | LC CDR3 | [symbol] |
| 15 | wild type | | MRPSGTAGAALLALLAALCPASRALEEKKVCQGTSNKL |
| | human | | TQLGTFEDHFLSLQRMFNNCEVVLGNLEITYVQRNYDL |
| | EGFR | | SFLKTIQEVAGYVLIALNTVERIPLENLQIIRGNMYYENS |
| | | | YALAVLSNYDANKTGLKELPMRNLQEILHGAVRFSNNP |
| | | | ALCNVESIQWRDIVSSDFLSNMSMDFQNHLGSCQKCDP |
| | | | SCPNGSCWGAGEENCQKLTKIICAQQCSGRCRGKSPSD |
| | | | CCHNQCAAGCTGPRESDCLVCRKFRDEATCKDTCPPLM |
| | | | LYNPTTYQMDVNPEGKYSFGATCVKKCPRNYVVTDHG |
| | | | SCVRACGADSYEMEEDGVRKCKKCEGPCRKVCNGIGIG |
| | | | EFKDSLSINATNIKHFKNCTSISGDLHILPVAFRGDSFTHT |
| | | | PPLDPQELDILKTVKEITGFLLIQAWPENRTDLHAFENLE |
| | | | IIRGRTKQHGQFSLAVVSLNITSLGLRSLKEISDGDVIISG |
| | | | NKNLCYANTINWKKLFGTSGQKTKIISNRGENSCKATG |
| | | | QVCHALCSPEGCWGPEPRDCVSCRNVSRGRECVDKCN |
| | | | LLEGEPREFVENSECIQCHPECLPQAMNITCTGRGPDNCI |
| | | | QCAHYIDGPHCVKTCPAGVMGENNTLVWKYADAGHV |
| | | | CHLCHPNCTYGCTGPGLEGCPTNGPKIPSIATGMVGALL |
| | | | LLLVVALGIGLFMRRRHIVRKRTLRRLLQERELVEPLTP |
| | | | SGEAPNQALLRILKETEFKKIKVLGSGAFGTVYKGLWIP |
| | | | EGEKVKIPVAIKELREATSPKANKEILDEAYVMASVDNP |
| | | | HVCRLLGICLTSTVQLITQLMPFGCLLDYVREHKDNIGS |
| | | | QYLLNWCVQIAKGMNYLEDRRLVHRDLAARNVLVKTP |
| | | | QHVKITDFGLAKLLGAEEKEYHAEGGKVPIKWMALESI |
| | | | LHRIYTHQSDVWSYGVTVWELMTFGSKPYDGIPASEISS |
| | | | ILEKGERLPQPPICTIDVYMIMVKCWMIDADSRPKFRELI |
| | | | IEFSKMARDPQRYLVIQGDERMHLPSPTDSNFYRALMD |
| | | | EEDMDDVVDADEYLIPQQGFFSSPSTSRTPLLSSLSATSN |
| | | | NSTVACIDRNGLQSCPIKEDSFLQRYSSDPTGALTEDSID |
| | | | DTFLPVPEYINQSVPKRPAGSVQNPVYHNQPLNPAPSRD |
| | | | PHYQDPHSTAVGNPEYLNTVQPTCVNSTFDSPAHWAQ |
| | | | KGSHQISLDNPDYQQDFFPKEAKPNGIFKGSTAENAEYL |
| | | | RVAPQSSEFIGA |
| 16 | truncated | | MRPSGTAGAALLALLAALCPASRALEEKKVCQGTSNKLT |
| | wild type | | QLGTFEDHFLSLQRMFNNCEVVLGNLEITYVQRNYDLSFL |
| | ECD of | | KTIQEVAGYVLIALNTVERIPLENLQIIRGNMYYENSYALA |
| | the EGFR | | VLSNYDANKTGLKELPMRNLQEILHGAVRFSNNPALCNV |
| | (EGFR (1- | | ESIQWRDIVSSDFLSNMSMDFQNHLGSCQKCDPSCPNGSC |
| | 525)) | | WGAGEENCQKLTKIICAQQCSGRCRGKSPSDCCHNQCAA |
| | | | GCTGPRESDCLVCRKFRDEATCKDTCPPLMLYNPTTYQM |
| | | | DVNPEGKYSFGATCVKKCPRNYVVTDHGSCVRACGADSY |
| | | | EMEEDGVRKCKKCEGPCRKVCNGIGIGEFKDSLSINATNI |
| | | | KHFKNCTSISGDLHILPVAFRGDSFTHTPPLDPQELDILKT |
| | | | VKEITGFLLIQAWPENRTDLHAFENLEIIRGRTKQHGQFS |
| | | | LAVVSLNITSLGLRSLKEISDGDVIISGNKNLCYANTINWK |
| | | | KLFGTSGQKTKIISNRGENSCKATGQVCHALCSPEGCWG |
| | | | PEPRDCVS |
| 17 | ECD of | | MRPSGTAGAA LLALLAALCP ASRALEEKKV CQGTSNKLTQ |
| | human | | LGTFEDHFLS LQRMFNNCEV VLGNLEITYV QRNYDLSFLK |
| | EGFR | | TIQEVAGYVL IALNTVERIP LENLQIIRGN MYYENSYALA |
| | | | VLSNYDANKT GLKELPMRNL QEILHGAVRF SNNPALCNVE |
| | | | SIQWRDIVSS DFLSNMSMDF QNHLGSCQKC DPSCPNGSCW |
| | | | GAGEENCQKL TKIICAQQCS GRCRGKSPSD CCHNQCAAGC |
| | | | TGPRESDCLV CRKFRDEATC KDTCPPLMLY NPTTYQMDVN |
| | | | PEGKYSFGAT CVKKCPRNYV VTDHGSCVRACGADSYEMEE |
| | | | DGVRKCKKCEGPCRKVCNGI GIGEFKDSLS INATNIKHFK |
| | | | NCTSISGDLH ILPVAFRGDS FTHTPPLDPQ ELDILKTVKE |
| | | | ITGFLLIQAW PENRTDLHAF ENLEIIRGRT KQHGQFSLAV |
| | | | VSLNITSLGL RSLKEISDGD VIISGNKNLC YANTINWKKL |
| | | | FGTSGQKTKI ISNRGENSCK ATGQVCHALC SPEGCWGPEP |
| | | | RDCVSCRNVS RGRECVDKCN LLEGEPREFV ENSECIQCHP |
| | | | ECLPQAMNIT CTGRGPDNCI QCAHYIDGPH CVKTCPAGVM |
| | | | GENNTLVWKY ADAGHVCHLC HPNCTYGCTG |
| | | | PGLEGCPTNG PKIPS |
| 18 | EGFRvIII | | MRPSGTAGAALLALLAALCPASRALEEKKGNYVVTDHGSC |
| | | | VRACGADSYEMEEDGVRKCKKCEGPCRKVCNGIGIGEFK |
| | | | DSLSINATNIKHFKNCTSISGDLHILPVAFRGDSFTHTPPLD |
| | | | PQELDILKTVKEITGFLLIQAWPENRTDLHAFENLEIIRGR |
| | | | TKQHGQFSLAVVSLNITSLGLRSLKEISDGDVIISGNKNLC |
| | | | YANTINWKKLFGTSGQKTKIISNRGENSCKATGQVCHAL |
| | | | CSPEGCWGPEPRDCVSCRNVSRGRECVDKCNLLEGEPRE |
| | | | FVENSECIQCHPECLPQAMNITCTGRGPDNCIQCAHYIDG |
| | | | PHCVKTCPAGVMGENNTLVWKYADAGHVCHLCHPNCT |
| | | | YGCTGPGLEGCPTNGPKIPSIATGMVGALLLLLVVALGIGLF |
| | | | MRRRHIVRKRTLRRLLQERELVEPLTPSGEAPNQALLRILKET |
| | | | EFKKIKVLGSGAFGTVYKGLWIPEGEKVKIPVAIKELREATSP |
| | | | KANKEILDEAYVMASVDNPHVCRLLGICLTSTVQLITQLMPF |
| | | | GCLLDYVREHKDNIGSQYLLNWCVQIAKGMNYLEDRRLVH |
| | | | RDLAARNVLVKTPQHVKITDFGLAKLLGAEEKEYHAEGGKV |
| | | | PIKWMALESILHRIYTHQSDVWSYGVTVWELMTFGSKPYDGI |
| | | | PASEISSILEKGERLPQPPICTIDVYMIMVKCWMIDADSRPKFR |
| | | | ELIIEFSKMARDPQRYLVIQGDERMHLPSPTDSNFYRALMDEE |
| | | | DMDDVVDADEYLIPQQGFFSSPSTSRTPLLSSLSATSNNSTVA |
| | | | CIDRNGLQSCPIKEDSFLQRYSSDPTGALTEDSIDDTFLPVPEY |
| | | | INQSVPKRPAGSVQNPVYHNQPLNPAPSRDPHYQDPHSTAVG |
| | | | NPEYLNTVQPTCVNSTFDSPAHWAQKGSHQISLDNPDYQQD |
| | | | FFPKEAKPNGIFKGSTAENAEYLRVAPQSSEFIGA |
| 19 | EGFRvIII | | LEEKKGNYVVTDHGSCVRACGADSYEMEEDGVRKCKKC |
| | ECD | | EGPCRKVCNGIGIGEFKDSLSINATNIKHFKNCTSISGDLHI |
| | | | LPVAFRGDSFTHTPPLDPQELDILKTVKEITGFLLIQAWPE |
| | | | NRTDLHAFENLEIIRGRTKQHGQFSLAVVSLNITSLGLRSL |
| | | | KEISDGDVIISGNKNLCYANTINWKKLFGTSGQKTKIISNR |
| | | | GENSCKATGQVCHALCSPEGCWGPEPRDCVSCRNVSRGR |
| | | | ECVDKCNLLEGEPREFVENSECIQCHPECLPQAMNITCTG |
| | | | RGPDNCIQCAHYIDGPHCVKTCPAGVMGENNTLVWKYA |
| | | | DAGHVCHLCHPNCTYGCTGPGLEGCPTNGP |
| 20 | Binding | | CGADSYEMEEDGVRKC |
| | epitope | | |
| | (amino | | |
| | acid | | |
| | residues | | |
| | 287-302 of | | |
| | the mature | | |
| | form of | | |
| | hEGFR) | | |
| 21 | AM2B | | EVQLQESGPGLVKPSQTLSLTCTVS [symbol] WIRQPPG |
| | Heavy | | KGLEWMG [symbol] RITISRDTSKNQFFLKLNSV |
| | Chain | | TAADTATYYCVT [symbol] WGQGTLVTVSSASTKGPSVFPL |
| | | | APSSKSTSGGTAALGCLVKDYFPEPVTVSWNSGALTSGVHTF |
| | | | PAVLQSSGLYSLSSVVTVPSSSLGTQTYICNVNHKPSNTKVDK |
| | | | KVEPKSCDKTHTCPPCPAPELLGGPSVFLFPPKPKDTLMISRTP |
| | | | EVTCVVVDVSHEDPEVKFNWYVDGVEVHNAKTKPREEQYN |
| | | | STYRVVSVLTVLHQDWLNGKEYKCKVSNKALPAPIEKTISKA |
| | | | KGQPREPQVYTLPPSREEMTKNQVSLTCLVKGFYPSDIAVEW |
| | | | ESNGQPENNYKTTPPVLDSDGSFFLYSKLTVDKSRWQQGNV |
| | | | FSCSVMHEALHNHYTQKSLSLSPGK |
| 2 | AM2B/ | CDR1 | [symbol] |
| | AM2 HC | | |
| | CDR1 | | |
| 3 | AM2B/ | CDR2 | [symbol] |
| | AM2 HC | | |
| | CDR2 | | |
| 4 | AM2B/ | CDR3 | [symbol] |
| | AM2 HC | | |
| | CDR3 | | |
| 22 | AM2/ | | EVQLQESGPGLVKPSQTLSLTCTVS [symbol] WIRQPPG |
| | AM2B VH | | KGLEWMG [symbol] RITISRDTSKNQFFLKLNSV |
| | | | TAADTATYYCVT [symbol] WGQGTLVTVSS |
| 23 | AM2/ | | DIQMTQSPSSMSVSVGDRVTITC [symbol] WLQQKPGK |
| | AM2B VL | | SFKGLIY [symbol] GVPSRFSGSGSGTDYTLTISSLQPEDFAT |
| | | | YYC [symbol] FGGGTKLEIK |
| 24 | AM2B | | DIQMTQSPSSMSVSVGDRVTITC [symbol] WLQQKPGK |
| | Light | | SFKGLIY [symbol] GVPSRFSGSGSGTDYTLTISSLQPEDFAT |
| | Chain | | YYC [symbol] FGGGTKLEIKRTVAAPSVFIFPPSDEQLKSG |
| | | | TASVVCLLNNFYPREAKVQWKVDNALQSGNSQESVTEQDSK |
| | | | DSTYSLSSTLTLSKADYEKHKVYACEVTHQGLSSPVTKSFNR |
| | | | GEC |
| 6 | AM2B/ | CDR1 | HSSQDINSNIG |
| | AM2 | | |
| | LCDR1 | | |
| 7 | AM2B/ | CDR2 | HGTNLDD |
| | AM2 | | |
| | LCDR2 | | |
| 8 | AM2B/ | CDR3 | VQYAQFPWT |
| | AM2 | | |
| | LCDR3 | | |
| 29 | MSL109 | Heavy | EEQVLESGGGLVKPGGSLRLSCAASGFTFSPYSVFWVRQAPG |
| | hIgG1 | Chain | KGLEWVSSINSDSTYKYYADSVKGRFTISRDNAENSIFLQMN |
| | Heavy | | SLRAEDTAVYYCARDRSYYAFSSGSLSDYYYGLDVWGQGTT |
| | Chain | | VIVSSASTKGPSVFPLAPSSKSTSGGTAALGCLVKDYFPEPVT |
| | | | VSWNSGALTSGVHTFPAVLQSSGLYSLSSVVTVPSSSLGTQT |
| | | | YICNVNHKPSNTKVDKKVEPKSCDKTHTCPPCPAPELLGGPS |
| | | | VFLFPPKPKDTLMISRTPEVTCVVVDVSHEDPEVKFNWYVDG |
| | | | VEVHNAKTKPREEQYNSTYRVVSVLTVLHQDWLNGKEYKC |
| | | | KVSNKALPAPIEKTISKAKGQPREPQVYTLPPSREEMTKNQVS |
| | | | LTCLVKGFYPSDIAVEWESNGQPENNYKTTPPVLDSDGSFFL |
| | | | YSKLTVDKSRWQQGNVFSCSVMHEALHNHYTQKSLSLSPGK |
| 30 | MSL109 | Light | DIVMTQSPLSLSVTPGEPASISCRSSQSLLHTNGYNYLDWYVQ |
| | light | Chain | KPGQSPQLLIYLASNRASGVPDRFSGSGSGTDFTLKISRVETE |
| | chain | | DVGVYYCMQALQIPRTFGQGTKVEIKRTVAAPSVFIFPPSDEQ |
| | | | LKSGTASVVCLLNNFYPREAKVQWKVDNALQSGNSQESVTE |
| | | | QDSKDSTYSLSSTLTLSKADYEKHKVYACEVTHQGLSSPVTK |
| | | | SFNRGEC |

We claim:

1. An anti-human epidermal growth factor receptor (hEGFR) antibody-drug conjugate comprising the following structure: 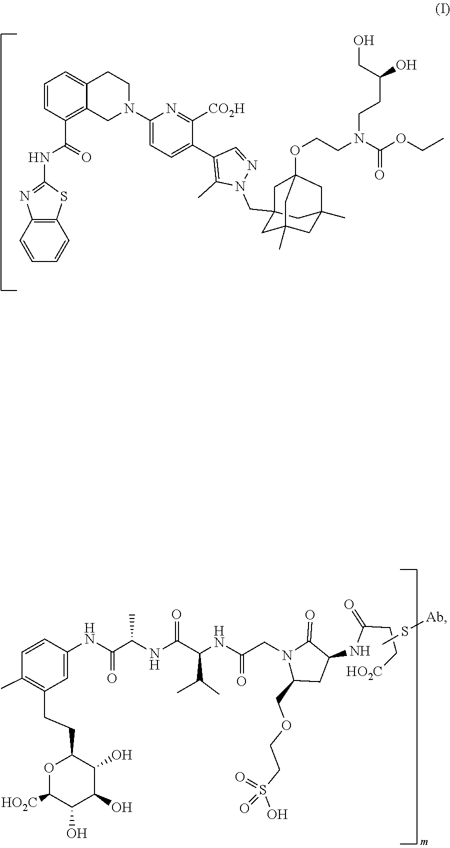
wherein Ab is an IgG1 anti-hEGFR antibody comprising a heavy chain comprising the amino acid sequence set forth as SEQ ID NO: 1 and a light chain comprising the amino acid sequence set forth as SEQ ID NO: 5; and wherein m is 2.

2. The antibody-drug conjugate of claim 1, wherein the structure of formula (I) is conjugated to antibody Ab through C220 of the heavy chain.

3. The ADC of claim 1, comprising the structure of formula (II):

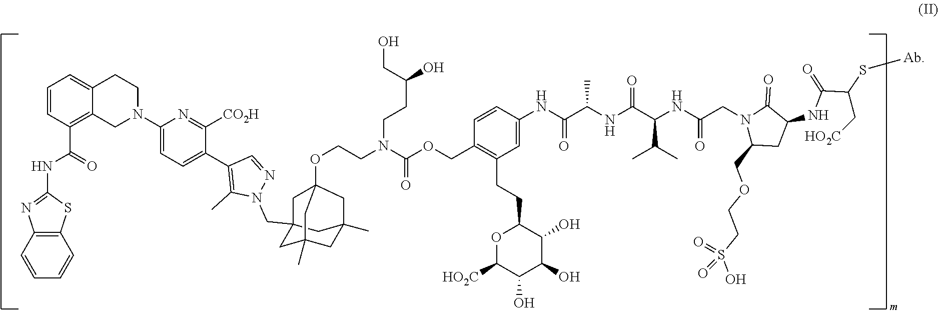

4. The ADC of claim 1, comprising the structure of formula (III):

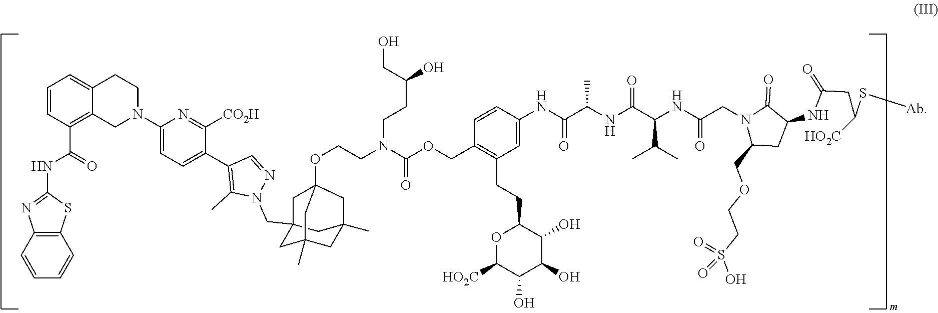

5. A method of producing an antibody drug conjugate (ADC) comprising a step of conjugating a monoclonal human IgG₁ anti-EGFR antibody with a synthon comprising the structure: 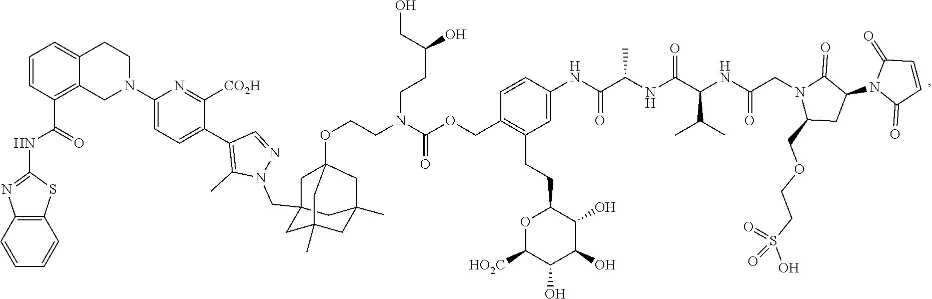 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CC[C@H](O)CO)C(=O)OCc1ccc(NC(=O)[C@H](C)NC(=O)[C@@H](NC(=O)CN4C(=O)[C@@H](N5C(=O)C=CC5=O)C[C@H]4COCCS(=O)(=O)O)C(C)C)cc1CC[C@@H]1O[C@H](C(=O)O)[C@@H](O)[C@H](O)[C@H]1O)(C3)C2]
to form an antibody-drug conjugate comprising a drug-linker conjugated to the anti-EGFR antibody, wherein the anti-EGFR antibody comprises a heavy chain comprising the amino acid sequence set forth as SEQ ID NO: 1 and a light chain comprising the amino acid sequence set forth as SEQ ID NO: 5; and wherein said drug-linker is conjugated to said antibody through C220 of the heavy chain.

6. A process for the preparation of an anti-human Epidermal Growth Factor Receptor antibody-drug conjugate comprising the following structure: 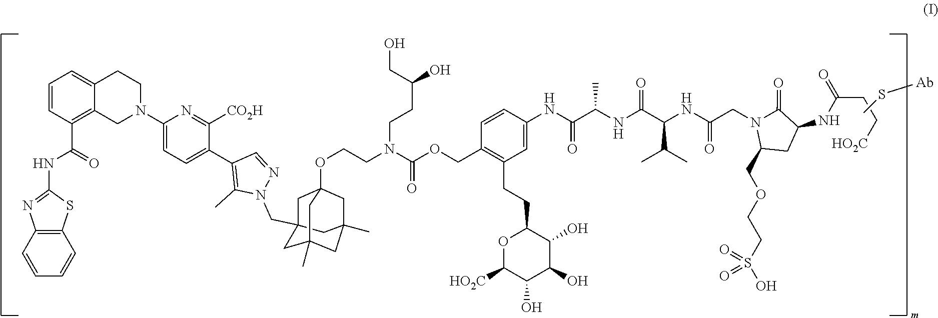
wherein Ab is an IgG1 anti-human epidermal growth factor receptor antibody comprising a heavy chain comprising the amino acid sequence set forth as SEQ ID NO: 1 and a light chain comprising the amino acid sequence set forth as SEQ ID NO: 5; and wherein m is 2; the method comprising the process comprising:
treating an antibody in a buffered aqueous solution with an effective amount of a disulfide reducing agent for about 16-24 hours;
adding to the reduced antibody solution a solution of dimethyl acetamide comprising a synthon having the following structure: 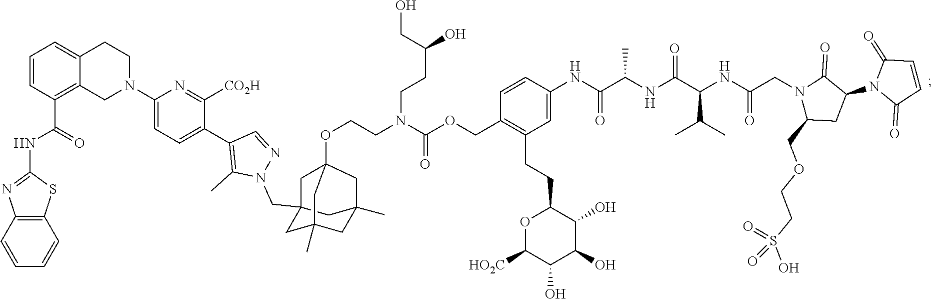 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCN(CC[C@H](O)CO)C(=O)OCc1ccc(NC(=O)[C@H](C)NC(=O)[C@@H](NC(=O)CN4C(=O)[C@@H](N5C(=O)C=CC5=O)C[C@H]4COCCS(=O)(=O)O)C(C)C)cc1CC[C@@H]1O[C@H](C(=O)O)[C@@H](O)[C@H](O)[C@H]1O)(C3)C2]
allowing the reaction to run to form the ADC;
wherein the mass is shifted by 18±2 amu for each hydrolysis of a succinimide to a succinamide as measured by electron spray mass spectrometry; and
wherein the ADC is optionally purified by hydrophobic interaction chromatography.

7. An antibody-drug conjugate prepared according to any one of claims 5 or 6.

8. A method of treating non-small cell lung cancer, the method comprising administering a therapeutically effective amount of the antibody-drug conjugate of any one of claims 1-4 to a patient in need thereof.

9. A pharmaceutical composition comprising an effective amount of the antibody-drug conjugate of any one of claims 1-4 and a pharmaceutically acceptable carrier.


SEQUENCE LISTING (32 sequences)

SEQ ID NO: 1 - protein, 446 aa, synthetic construct
  REGION 1..446: note = Synthetic Polypeptide
EVQLQESGPGLVKPSQTLSLTCTVSGYSISNDFAWNWIRQPPGKGLEWMGYISYKGNTRYQPSLKSRITISRDTSKNQFFLKLNSVTAADTATYYCVTASRGFPWWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 11 aa, synthetic construct
  REGION 1..11: note = Synthetic Polypeptide
GYSISNDFAWN

SEQ ID NO: 3 - protein, 16 aa, synthetic construct
  REGION 1..16: note = Synthetic Polypeptide
YISYKGNTRYQPSLKS

SEQ ID NO: 4 - protein, 7 aa, synthetic construct
  REGION 1..7: note = Synthetic Polypeptide
ASRGFPW

SEQ ID NO: 5 - protein, 214 aa, synthetic construct
  REGION 1..214: note = Synthetic Polypeptide
DIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 6 - protein, 11 aa, synthetic construct
  REGION 1..11: note = Synthetic Polypeptide
HSSQDINSNIG

SEQ ID NO: 7 - protein, 7 aa, synthetic construct
  REGION 1..7: note = Synthetic Polypeptide
HGTNLDD

SEQ ID NO: 8 - protein, 9 aa, synthetic construct
  REGION 1..9: note = Synthetic Polypeptide
VQYAQFPWT

SEQ ID NO: 9 - protein, 446 aa, synthetic construct
  REGION 1..446: note = Synthetic Polypeptide
EVQLQESGPGLVKPSQTLSLTCTVSGYSISNDFAWNWIRQLPGKGLEWMGYISYKGNTRYQPSLKSRITISRDTSKNQFFLKLNSVTAADTATYYCVTASRGLPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 7 aa, synthetic construct
  REGION 1..7: note = Synthetic Polypeptide
ASRGLPY

SEQ ID NO: 11 - protein, 214 aa, synthetic construct
  REGION 1..214: note = Synthetic Polypeptide
DIQMTQSPSSMSVSVGDRVTITCHSSQDITYNIGWLQQKPGKSFKGLIYHGANLDDGVPSRFSGSGSGTDYTLTISSLQPEDFATYYCVQYDEFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 12 - protein, 11 aa, synthetic construct
  REGION 1..11: note = Synthetic Polypeptide
HSSQDITYNIG

SEQ ID NO: 13 - protein, 7 aa, synthetic construct
  REGION 1..7: note = Synthetic Polypeptide
HGANLDD

SEQ ID NO: 14 - protein, 9 aa, synthetic construct
  REGION 1..9: note = Synthetic Polypeptide
VQYDEFPWT

SEQ ID NO: 15 - protein, 1210 aa, synthetic construct [sequence omitted]

SEQ ID NO: 16 - protein, 525 aa, synthetic construct
  REGION 1..525: note = Synthetic Polypeptide
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVS

SEQ ID NO: 17 - protein, 645 aa, synthetic construct
  REGION 1..645: note = Synthetic Polypeptide
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPS

SEQ ID NO: 18 - protein, 943 aa, synthetic construct
  REGION 1..943: note = Synthetic Polypeptide
MRPSGTAGAALLALLAALCPASRALEEKKGNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 19 - protein, 350 aa, synthetic construct
  REGION 1..350: note = Synthetic Polypeptide
LEEKKGNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGP

SEQ ID NO: 20 - protein, 16 aa, synthetic construct
  REGION 1..16: note = Synthetic Polypeptide
CGADSYEMEEDGVRKC

SEQ ID NO: 21 - protein, 446 aa, synthetic construct
  REGION 1..446: note = Synthetic Polypeptide
EVQLQESGPGLVKPSQTLSLTCTVSGYSISNDFAWNWIRQPPGKGLEWMGYISYKGNTRYQPSLKSRITISRDTSKNQFFLKLNSVTAADTATYYCVTASRGFPWWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 116 aa, synthetic construct
  REGION 1..116: note = Synthetic Polypeptide
EVQLQESGPGLVKPSQTLSLTCTVSGYSISNDFAWNWIRQPPGKGLEWMGYISYKGNTRYQPSLKSRITISRDTSKNQFFLKLNSVTAADTATYYCVTASRGFPWWGQGTLVTVSS

SEQ ID NO: 23 - protein, 107 aa, synthetic construct
  REGION 1..107: note = Synthetic Polypeptide
DIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIK

SEQ ID NO: 24 - protein, 214 aa, synthetic construct
  REGION 1..214: note = Synthetic Polypeptide
DIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 19 aa, synthetic construct
  REGION 1..19: note = Synthetic Polypeptide
MEFGLSWLFLVAILKGVQC

SEQ ID NO: 26 - protein, 22 aa, synthetic construct
  REGION 1..22: note = Synthetic Polypeptide
MDMRVPAQLLGLLLLWFPGSRC

SEQ ID NO: 27 - protein, 673 aa, synthetic construct
  REGION 1..673: note = Synthetic Polypeptide
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSLESRGPFEQKLISEEDLNMHTGHHHHHH

SEQ ID NO: 28 - protein, 553 aa, synthetic construct
  REGION 1..553: note = Synthetic Polypeptide
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSLESRGPFEQKLISEEDLNMHTGHHHHHH

SEQ ID NO: 29 - protein, 460 aa, synthetic construct
  REGION 1..460: note = Synthetic Polypeptide
EEQVLESGGGLVKPGGSLRLSCAASGFTFSPYSVFWVRQAPGKGLEWVSSINSDSTYKYYADSVKGRFTISRDNAENSIFLQMNSLRAEDTAVYYCARDRSYYAFSSGSLSDYYYGLDVWGQGTTVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 219 aa, synthetic construct
  REGION 1..219: note = Synthetic Polypeptide
DIVMTQSPLSLSVTPGEPASISCRSSQSLLHTNGYNYLDWYVQKPGQSPQLLIYLASNRASGVPDRFSGSGSGTDFTLKISRVETEDVGVYYCMQALQIPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 406 aa, synthetic construct
  REGION 1..406: note = Synthetic Polypeptide
MRPSGTAGAALLALLAALCPASRALEEKKGNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSLESRGPFEQKLISEEDLNMHTGHHHHHH

SEQ ID NO: 32 - protein, 24 aa, synthetic construct
  REGION 1..24: note = Synthetic Polypeptide
MRPSGTAGAALLALLAALCPASRA